SEQUENCE LISTING (82 sequences)

SEQ ID NO: 1 - RNA, 50 nt, synthetic construct
  source 1..50: mol_type = other RNA
gaccaccccaaaaatgaaggggactaaaactttcgctgattttggggtcc

SEQ ID NO: 2 - RNA, 50 nt, synthetic construct
  source 1..50: mol_type = other RNA
gaccaccccaaaaatgaaggggactaaaacggtccaccaaacgtaatgcg

SEQ ID NO: 3 - RNA, 50 nt, synthetic construct
  source 1..50: mol_type = other RNA
gaccaccccaaaaatgaaggggactaaaactctggttactgccagttgaa

SEQ ID NO: 4 - RNA, 50 nt, synthetic construct
  source 1..50: mol_type = other RNA
gaccaccccaaaaatgaaggggactaaaactttgcggccaatgtttgtaa

SEQ ID NO: 5 - RNA, 50 nt, synthetic construct
  source 1..50: mol_type = other RNA
gaccaccccaaaaatgaaggggactaaaacgaagcgctgggggcaaattg

SEQ ID NO: 6 - RNA, 50 nt, synthetic construct
  source 1..50: mol_type = other RNA
gaccaccccaaaaatgaaggggactaaaacatgcgcgacattccgaagaa

SEQ ID NO: 7 - RNA, 50 nt, synthetic construct
  source 1..50: mol_type = other RNA
gaccaccccaaaaatgaaggggactaaaacttggtgtattcaaggctccc

SEQ ID NO: 8 - RNA, 50 nt, synthetic construct
  source 1..50: mol_type = other RNA
gaccaccccaaaaatgaaggggactaaaacggattgcgggtgccaatgtg

SEQ ID NO: 9 - RNA, 50 nt, synthetic construct
  source 1..50: mol_type = other RNA
gaccaccccaaaaatgaaggggactaaaactgtagcacgattgcagcatt

SEQ ID NO: 10 - RNA, 50 nt, synthetic construct
  source 1..50: mol_type = other RNA
gaccaccccaaaaatgaaggggactaaaactaagtgtaaaacccacaggg

SEQ ID NO: 11 - RNA, 50 nt, synthetic construct
  source 1..50: mol_type = other RNA
gaccaccccaaaaatgaaggggactaaaactaacctttccacataccgca

SEQ ID NO: 12 - RNA, 50 nt, synthetic construct
  source 1..50: mol_type = other RNA
gaccaccccaaaaatgaaggggactaaaactcagctgatgcacaatcgtt

SEQ ID NO: 13 - RNA, 50 nt, synthetic construct
  source 1..50: mol_type = other RNA
gaccaccccaaaaatgaaggggactaaaactctagcaggagaagttcccc

SEQ ID NO: 14 - RNA, 50 nt, synthetic construct
  source 1..50: mol_type = other RNA
gaccaccccaaaaatgaaggggactaaaactctgtcaagcagcagcaaag

SEQ ID NO: 15 - RNA, 50 nt, synthetic construct
  source 1..50: mol_type = other RNA
gaccaccccaaaaatgaaggggactaaaacctttgctgctgcttgacaga

SEQ ID NO: 16 - RNA, 50 nt, synthetic construct
  source 1..50: mol_type = other RNA
gaccaccccaaaaatgaaggggactaaaacaacgattgtgcatcagctga

SEQ ID NO: 17 - RNA, 50 nt, synthetic construct
  source 1..50: mol_type = other RNA
gaccaccccaaaaatgaaggggactaaaacgacattttgctctcaagctg

SEQ ID NO: 18 - RNA, 50 nt, synthetic construct
  source 1..50: mol_type = other RNA
gaccaccccaaaaatgaaggggactaaaacgttcctggtccccaaaattt

SEQ ID NO: 19 - RNA, 50 nt, synthetic construct
  source 1..50: mol_type = other RNA
gaccaccccaaaaatgaaggggactaaaactggcacctgtgtaggtcaac

SEQ ID NO: 20 - RNA, 50 nt, synthetic construct
  source 1..50: mol_type = other RNA
gaccaccccaaaaatgaaggggactaaaactccatgccaatgcgcgacat

SEQ ID NO: 21 - RNA, 50 nt, synthetic construct
  source 1..50: mol_type = other RNA
gaccaccccaaaaatgaaggggactaaaacctattaactattaacgtacc

SEQ ID NO: 22 - RNA, 50 nt, synthetic construct
  source 1..50: mol_type = other RNA
gaccaccccaaaaatgaaggggactaaaactattgcagcagtacgcacac

SEQ ID NO: 23 - RNA, 50 nt, synthetic construct
  source 1..50: mol_type = other RNA
gaccaccccaaaaatgaaggggactaaaacagcgcagtaaggatggctag

SEQ ID NO: 24 - RNA, 50 nt, synthetic construct
  source 1..50: mol_type = other RNA
gaccaccccaaaaatgaaggggactaaaacgtaactagcaagaataccac

SEQ ID NO: 25 - RNA, 52 nt, synthetic construct
  source 1..52: mol_type = other RNA
tagaccaccccaaaaatgaaggggactaaaacggtccaccaaacgtaatgcg

SEQ ID NO: 26 - RNA, 52 nt, synthetic construct; identical to SEQ ID NO: 25
  source 1..52: mol_type = other RNA

SEQ ID NO: 27 - RNA, 52 nt, synthetic construct
  source 1..52: mol_type = other RNA
tagaccaccccaaaaatgaaggggactaaaaccgcattacgtttggtggacc

SEQ ID NO: 28 - RNA, 52 nt, synthetic construct
  source 1..52: mol_type = other RNA
tagaccaccccaaaaatgaaggggactaaaacttacaaacattggccgcaaa

SEQ ID NO: 29 - RNA, 52 nt, synthetic construct
  source 1..52: mol_type = other RNA
tagaccaccccaaaaatgaaggggactaaaacaaactacgtcatcaagccaa

SEQ ID NO: 30 - RNA, 51 nt, synthetic construct
  source 1..51: mol_type = other RNA
tagaccaccccaaaaatgaaggggactaaaaccacagtcataatctatgta

SEQ ID NO: 31 - RNA, 52 nt, synthetic construct
  source 1..52: mol_type = other RNA
tagaccaccccaaaaatgaaggggactaaaactcacacttttctaatagcat

SEQ ID NO: 32 - RNA, 51 nt, synthetic construct
  source 1..51: mol_type = other RNA
tagaccaccccaaaaatgaaggggactaaaactgtaagataacacactgac

SEQ ID NO: 33 - RNA, 52 nt, synthetic construct
  source 1..52: mol_type = other RNA
tagaccaccccaaaaatgaaggggactaaaacttaattgtgtacaaaaactg

SEQ ID NO: 34 - RNA, 52 nt, synthetic construct
  source 1..52: mol_type = other RNA
tagaccaccccaaaaatgaaggggactaaaaccagttgtgatgattcctaag

SEQ ID NO: 35 - RNA, 52 nt, synthetic construct
  source 1..52: mol_type = other RNA
tagaccaccccaaaaatgaaggggactaaaacagcgcagtaaggatggctag

SEQ ID NO: 36 - RNA, 52 nt, synthetic construct
  source 1..52: mol_type = other RNA
tagaccaccccaaaaatgaaggggactaaaacgcttgtgttacattgtatgc

SEQ ID NO: 37 - RNA, 52 nt, synthetic construct
  source 1..52: mol_type = other RNA
tagaccaccccaaaaatgaaggggactaaaacaatttgatggcacctgtgta

SEQ ID NO: 38 - RNA, 52 nt, synthetic construct; identical to SEQ ID NO: 29
  source 1..52: mol_type = other RNA

SEQ ID NO: 39 - RNA, 52 nt, synthetic construct
  source 1..52: mol_type = other RNA
tagaccaccccaaaaatgaaggggactaaaaccacagtcataatctatgtta

SEQ ID NO: 40 - RNA, 52 nt, synthetic construct; identical to SEQ ID NO: 31
  source 1..52: mol_type = other RNA

SEQ ID NO: 41 - RNA, 51 nt, synthetic construct; identical to SEQ ID NO: 32
  source 1..51: mol_type = other RNA

SEQ ID NO: 42 - RNA, 52 nt, synthetic construct; identical to SEQ ID NO: 33
  source 1..52: mol_type = other RNA

SEQ ID NO: 43 - RNA, 52 nt, synthetic construct; identical to SEQ ID NO: 34
  source 1..52: mol_type = other RNA

SEQ ID NO: 44 - RNA, 52 nt, synthetic construct
  source 1..52: mol_type = other RNA
tagaccaccccaaaaatgaaggggactaaaactgataagacctcctccacgg

SEQ ID NO: 45 - RNA, 52 nt, synthetic construct
  source 1..52: mol_type = other RNA
tagaccaccccaaaaatgaaggggactaaaaccaaaagcctcaacacgtaga

SEQ ID NO: 46 - RNA, 52 nt, synthetic construct
  source 1..52: mol_type = other RNA
tagaccaccccaaaaatgaaggggactaaaaccatgttgaaacaagtaactc

SEQ ID NO: 47 - RNA, 52 nt, synthetic construct
  source 1..52: mol_type = other RNA
tagaccaccccaaaaatgaaggggactaaaactcaacgatgtaagaagactg

SEQ ID NO: 48 - RNA, 52 nt, synthetic construct
  source 1..52: mol_type = other RNA
tagaccaccccaaaaatgaaggggactaaaacacactatcaacgatgtaaga

SEQ ID NO: 49 - RNA, 52 nt, synthetic construct
  source 1..52: mol_type = other RNA
tagaccaccccaaaaatgaaggggactaaaactaaccatccactgaatatgt

SEQ ID NO: 50 - RNA, 52 nt, synthetic construct
  source 1..52: mol_type = other RNA
tagaccaccccaaaaatgaaggggactaaaacaaacagtaaggccgttaaac

SEQ ID NO: 51 - RNA, 52 nt, synthetic construct
  source 1..52: mol_type = other RNA
tagaccaccccaaaaatgaaggggactaaaactccaattgtgaagattctca

SEQ ID NO: 52 - RNA, 52 nt, synthetic construct
  source 1..52: mol_type = other RNA
tagaccaccccaaaaatgaaggggactaaaacgactaaagcataaagataga

SEQ ID NO: 53 - RNA, 52 nt, synthetic construct
  source 1..52: mol_type = other RNA
tagaccaccccaaaaatgaaggggactaaaacaaaattatcgaagcttgcgt

SEQ ID NO: 54 - RNA, 52 nt, synthetic construct
  source 1..52: mol_type = other RNA
tagaccaccccaaaaatgaaggggactaaaaccagtacatcaaacgaatttg

SEQ ID NO: 55 - RNA, 52 nt, synthetic construct
  source 1..52: mol_type = other RNA
tagaccaccccaaaaatgaaggggactaaaacggaaatacaaatgatataag

SEQ ID NO: 56 - RNA, 52 nt, synthetic construct
  source 1..52: mol_type = other RNA
tagaccaccccaaaaatgaaggggactaaaacaatttccatttgactcctgg

SEQ ID NO: 57 - RNA, 52 nt, synthetic construct
  source 1..52: mol_type = other RNA
tagaccaccccaaaaatgaaggggactaaaaccaatctttaaagagtcctgt

SEQ ID NO: 58 - RNA, 52 nt, synthetic construct
  source 1..52: mol_type = other RNA
tagaccaccccaaaaatgaaggggactaaaacttaaattgatctccaggcgg

SEQ ID NO: 59 - RNA, 52 nt, synthetic construct
  source 1..52: mol_type = other RNA
tagaccaccccaaaaatgaaggggactaaaacgaagtagactaaagcataaa

SEQ ID NO: 60 - RNA, 52 nt, synthetic construct
  source 1..52: mol_type = other RNA
tagaccaccccaaaaatgaaggggactaaaaccaccggtgatccaatttatt

SEQ ID NO: 61 - RNA, 50 nt, synthetic construct
  source 1..50: mol_type = other RNA
gaccaccccaaaaatgaaggggactaaaaccctctctggtaggaacacgc

SEQ ID NO: 62 - RNA, 50 nt, synthetic construct
  source 1..50: mol_type = other RNA
gaccaccccaaaaatgaaggggactaaaacaagatcctgataaagaacag

SEQ ID NO: 63 - RNA, 50 nt, synthetic construct
  source 1..50: mol_type = other RNA
gaccaccccaaaaatgaaggggactaaaacctttccatacaacttttgtt

SEQ ID NO: 64 - RNA, 46 nt, synthetic construct
  source 1..46: mol_type = other RNA
gcacagcagaaaatctctgcttacaaacattggccgcaaaccacag

SEQ ID NO: 65 - RNA, 10 nt, synthetic construct
  source 1..10: mol_type = other RNA
rtrtrtrtrt

SEQ ID NO: 66 - RNA, 10 nt, synthetic construct
  source 1..10: mol_type = other RNA
rtrtrtrtrt

SEQ ID NO: 67 - RNA, 49 nt, synthetic construct
  source 1..49: mol_type = other RNA
ccaccccaaaaatgaaggggactaaaacaggttcttgactaccgtaatt

SEQ ID NO: 68 - RNA, 49 nt, synthetic construct
  source 1..49: mol_type = other RNA
ccaccccaaaaatgaaggggactaaaacatcggatgtaaattatgccgt

SEQ ID NO: 69 - RNA, 49 nt, synthetic construct
  source 1..49: mol_type = other RNA
ccaccccaaaaatgaaggggactaaaacacagctcatcggtgtcctact

SEQ ID NO: 70 - RNA, 50 nt, synthetic construct
  source 1..50: mol_type = other RNA
gaccaccccaaaaatgaaggggactaaaacggttcttgactaccgtaatt

SEQ ID NO: 71 - protein, 1177 aa, Leptotrichia wadei
MKITKIDGVSHYKKQDKGILKKKWKDLDERKQREKIEARYNKQIESKIYKEFFRLKNKKRIEKEEDQNIKSLYFFIKELYLNEKNEEWELKNINLEILDDKERVIKGYKFKEDVYFFKEGYKEYYLRILFNNLIEKVQNENREKVRKNKEFLDLKEIFKKYKNRKIDLLLKSINNNKINLEYKKENVNEEIYGINPTNDREMTFYELLKEIIEKKDEQKSILEEKLDNFDITNFLENIEKIFNEETEINIIKGKVLNELREYIKEKEENNSDNKLKQIYNLELKKYIENNFSYKKQKSKSKNGKNDYLYLNFLKKIMFIEEVDEKKEINKEKFKNKINSNFKNLFVQHILDYGKLLYYKENDEYIKNTGQLETKDLEYIKTKETLIRKMAVLVSFAANSYYNLFGRVSGDILGTEVVKSSKTNVIKVGSHIFKEKMLNYFFDFEIFDANKIVEILESISYSIYNVRNGVGHFNKLILGKYKKKDINTNKRIEEDLNNNEEIKGYFIKKRGEIERKVKEKFLSNNLQYYYSKEKIENYFEVYEFEILKRKIPFAPNFKRIIKKGEDLFNNKNNKKYEYFKNFDKNSAEEKKEFLKTRNFLLKELYYNNFYKEFLSKKEEFEKIVLEVKEEKKSRGNINNKKSGVSFQSIDDYDTKINISDYIASIHKKEMERVEKYNEEKQKDTAKYIRDFVEEIFLTGFINYLEKDKRLHFLKEEFSILCNNNNNVVDFNININEEKIKEFLKENDSKTLNLYLFFNMIDSKRISEFRNELVKYKQFTKKRLDEEKEFLGIKIELYETLIEFVILTREKLDTKKSEEIDAWLVDKLYVKDSNEYKEYEEILKLFVDEKILSSKEAPYYATDNKTPILLSNFEKTRKYGTQSFLSEIQSNYKYSKVEKENIEDYNKKEEIEQKKKSNIEKLQDLKVELHKKWEQNKITEKEIEKYNNTTRKINEYNYLKNKEELQNVYLLHEMLSDLLARNVAFFNKWERDFKFIVIAIKQFLRENDKEKVNEFLNPPDNSKGKKVYFSVSKYKNTVENIDGIHKNFMNLIFLNNKFMNRKIDKMNCAIWVYFRNYIAHFLHLHTKNEKISLISQMNLLIKLFSYDKKVQNHILKSTKTLLEKYNIQINFEISNDKNEVFKYKIKNRLYSKKGKMLGKNNKLENEFLENVKAMLEYSE

SEQ ID NO: 72 - protein, 1285 aa, Herbinix hemicellulosilytica
MKLTRRRISGNSVDQKITAAFYRDMSQGLLYYDSEDNDCTDKVIESMDFERSWRGRILKNGEDDKNPFYMFVKGLVGSNDKIVCEPIDVDSDPDNLDILINKNLTGFGRNLKAPDSNDTLENLIRKIQAGIPEEEVLPELKKIKEMIQKDIVNRKEQLLKSIKNNRIPFSLEGSKLVPSTKKMKWLFKLIDVPNKTFNEKMLEKYWEIYDYDKLKANITNRLDKTDKKARSISRAVSEELREYHKNLRTNYNRFVSGDRPAAGLDNGGSAKYNPDKEEFLLFLKEVEQYFKKYFPVKSKHSNKSKDKSLVDKYKNYCSYKVVKKEVNRSIINQLVAGLIQQGKLLYYFYYNDTWQEDFLNSYGLSYIQVEEAFKKSVMTSLSWGINRLTSFFIDDSNTVKFDDITTKKAKEAIESNYFNKLRTCSRMQDHFKEKLAFFYPVYVKDKKDRPDDDIENLIVLVKNAIESVSYLRNRTFHFKESSLLELLKELDDKNSGQNKIDYSVAAEFIKRDIENLYDVFREQIRSLGIAEYYKADMISDCFKTCGLEFALYSPKNSLMPAFKNVYKRGANLNKAYIRDKGPKETGDQGQNSYKALEEYRELTWYIEVKNNDQSYNAYKNLLQLIYYHAFLPEVRENEALITDFINRTKEWNRKETEERLNTKNNKKHKNFDENDDITVNTYRYESIPDYQGESLDDYLKVLQRKQMARAKEVNEKEEGNNNYIQFIRDVVVWAFGAYLENKLKNYKNELQPPLSKENIGLNDTLKELFPEEKVKSPFNIKCRFSISTFIDNKGKSTDNTSAEAVKTDGKEDEKDKKNIKRKDLLCFYLFLRLLDENEICKLQHQFIKYRCSLKERRFPGNRTKLEKETELLAELEELMELVRFTMPSIPEISAKAESGYDTMIKKYFKDFIEKKVFKNPKTSNLYYHSDSKTPVTRKYMALLMRSAPLHLYKDIFKGYYLITKKECLEYIKLSNIIKDYQNSLNELHEQLERIKLKSEKQNGKDSLYLDKKDFYKVKEYVENLEQVARYKHLQHKINFESLYRIFRIHVDIAARMVGYTQDWERDMHFLFKALVYNGVLEERRFEAIFNNNDDNNDGRIVKKIQNNLNNKNRELVSMLCWNKKLNKNEFGAIIWKRNPIAHLNHFTQTEQNSKSSLESLINSLRILLAYDRKRQNAVTKTINDLLLNDYHIRIKWEGRVDEGQIYFNIKEKEDIENEPIIHLKHLHKKDCYIYKNSYMFDKQKEWICNGIKEEVYDKSILKCIGNLFKFDYEDKNKSSANPKHT

SEQ ID NO: 73 - protein, 1159 aa, Leptotrichia buccalis
MKVTKVGGISHKKYTSEGRLVKSESEENRTDERLSALLNMRLDMYIKNPSSTETKENQKRIGKLKKFFSNKMVYLKDNTLSLKNGKKENIDREYSETDILESDVRDKKNFAVLKKIYLNENVNSEELEVFRNDIKKKLNKINSLKYSFEKNKANYQKINENNIEKVEGKSKRNIIYDYYRESAKRDAYVSNVKEAFDKLYKEEDIAKLVLEIENLTKLEKYKIREFYHEIIGRKNDKENFAKIIYEEIQNVNNMKELIEKVPDMSELKKSQVFYKYYLDKEELNDKNIKYAFCHFVEIEMSQLLKNYVYKRLSNISNDKIKRIFEYQNLKKLIENKLLNKLDTYVRNCGKYNYYLQDGEIATSDFIARNRQNEAFLRNIIGVSSVAYFSLRNILETENENDITGRMRGKTVKNNKGEEKYVSGEVDKIYNENKKNEVKENLKMFYSYDFNMDNKNEIEDFFANIDEAISSIRHGIVHFNLELEGKDIFAFKNIAPSEISKKMFQNEINEKKLKLKIFRQLNSANVFRYLEKYKILNYLKRTRFEFVNKNIPFVPSFTKLYSRIDDLKNSLGIYWKTPKTNDDNKTKEIIDAQIYLLKNIYYGEFLNYFMSNNGNFFEISKEIIELNKNDKRNLKTGFYKLQKFEDIQEKIPKEYLANIQSLYMINAGNQDEEEKDTYIDFIQKIFLKGFMTYLANNGRLSLIYIGSDEETNTSLAEKKQEFDKFLKKYEQNNNIKIPYEINEFLREIKLGNILKYTERLNMFYLILKLLNHKELTNLKGSLEKYQSANKEEAFSDQLELINLLNLDNNRVTEDFELEADEIGKFLDFNGNKVKDNKELKKFDTNKIYFDGENIIKHRAFYNIKKYGMLNLLEKIADKAGYKISIEELKKYSNKKNEIEKNHKMQENLHRKYARPRKDEKFTDEDYESYKQAIENIEEYTHLKNKVEFNELNLLQGLLLRILHRLVGYTSIWERDLRFRLKGEFPENQYIEEIFNFENKKNVKYKGGQIVEKYIKFYKELHQNDEVKINKYSSANIKVLKQEKKDLYIRNYIAHFNYIPHAEISLLEVLENLRKLLSYDRKLKNAVMKSVVDILKEYGFVATFKIGADKKIGIQTLESEKIVHLKNLKKKKLMTDRNSEELCKLVKIMFEYKMEEKKSEN

SEQ ID NO: 74 - protein, 1120 aa, Leptotrichia seeligeri
MWISIKTLIHHLGVLFFCDYMYNRREKKIIEVKTMRITKVEVDRKKVLISRDKNGGKLVYENEMQDNTEQIMHHKKSSFYKSVVNKTICRPEQKQMKKLVHGLLQENSQEKIKVSDVTKLNISNFLNHRFKKSLYYFPENSPDKSEEYRIEINLSQLLEDSLKKQQGTFICWESFSKDMELYINWAENYISSKTKLIKKSIRNNRIQSTESRSGQLMDRYMKDILNKNKPFDIQSVSEKYQLEKLTSALKATFKEAKKNDKEINYKLKSTLQNHERQIIEELKENSELNQFNIEIRKHLETYFPIKKTNRKVGDIRNLEIGEIQKIVNHRLKNKIVQRILQEGKLASYEIESTVNSNSLQKIKIEEAFALKFINACLFASNNLRNMVYPVCKKDILMIGEFKNSFKEIKHKKFIRQWSQFFSQEITVDDIELASWGLRGAIAPIRNEIIHLKKHSWKKFFNNPTFKVKKSKIINGKTKDVTSEFLYKETLFKDYFYSELDSVPELIINKMESSKILDYYSSDQLNQVFTIPNFELSLLTSAVPFAPSFKRVYLKGFDYQNQDEAQPDYNLKLNIYNEKAFNSEAFQAQYSLFKMVYYQVFLPQFTTNNDLFKSSVDFILTLNKERKGYAKAFQDIRKMNKDEKPSEYMSYIQSQLMLYQKKQEEKEKINHFEKFINQVFIKGFNSFIEKNRLTYICHPTKNTVPENDNIEIPFHTDMDDSNIAFWLMCKLLDAKQLSELRNEMIKFSCSLQSTEEISTFTKAREVIGLALLNGEKGCNDWKELFDDKEAWKKNMSLYVSEELLQSLPYTQEDGQTPVINRSIDLVKKYGTETILEKLFSSSDDYKVSAKDIAKLHEYDVTEKIAQQESLHKQWIEKPGLARDSAWTKKYQNVINDISNYQWAKTKVELTQVRHLHQLTIDLLSRLAGYMSIADRDFQFSSNYILERENSEYRVTSWILLSENKNKNKYNDYELYNLKNASIKVSSKNDPQLKVDLKQLRLTLEYLELFDNRLKEKRNNISHFNYLNGQLGNSILELFDDARDVLSYDRKLKNAVSKSLKEILSSHGMEVTFKPLYQTNHHLKIDKLQPKKIHHLGEKSTVSSNQVSNEYCQLVRTLLTMK

SEQ ID NO: 75 - protein, 1154 aa, Paludibacter propionicigenes
MRVSKVKVKDGGKDKMVLVHRKTTGAQLVYSGQPVSNETSNILPEKKRQSFDLSTLNKTIIKFDTAKKQKLNVDQYKIVEKIFKYPKQELPKQIKAEEILPFLNHKFQEPVKYWKNGKEESFNLTLLIVEAVQAQDKRKLQPYYDWKTWYIQTKSDLLKKSIENNRIDLTENLSKRKKALLAWETEFTASGSIDLTHYHKVYMTDVLCKMLQDVKPLTDDKGKINTNAYHRGLKKALQNHQPAIFGTREVPNEANRADNQLSIYHLEVVKYLEHYFPIKTSKRRNTADDIAHYLKAQTLKTTIEKQLVNAIRANIIQQGKTNHHELKADTTSNDLIRIKTNEAFVLNLTGTCAFAANNIRNMVDNEQTNDILGKGDFIKSLLKDNTNSQLYSFFFGEGLSTNKAEKETQLWGIRGAVQQIRNNVNHYKKDALKTVFNISNFENPTITDPKQQTNYADTIYKARFINELEKIPEAFAQQLKTGGAVSYYTIENLKSLLTTFQFSLCRSTIPFAPGFKKVFNGGINYQNAKQDESFYELMLEQYLRKENFAEESYNARYFMLKLIYNNLFLPGFTTDRKAFADSVGFVQMQNKKQAEKVNPRKKEAYAFEAVRPMTAADSIADYMAYVQSELMQEQNKKEEKVAEETRINFEKFVLQVFIKGFDSFLRAKEFDFVQMPQPQLTATASNQQKADKLNQLEASITADCKLTPQYAKADDATHIAFYVFCKLLDAAHLSNLRNELIKFRESVNEFKFHHLLEIIEICLLSADVVPTDYRDLYSSEADCLARLRPFIEQGADITNWSDLFVQSDKHSPVIHANIELSVKYGTTKLLEQIINKDTQFKTTEANFTAWNTAQKSIEQLIKQREDHHEQWVKAKNADDKEKQERKREKSNFAQKFIEKHGDDYLDICDYINTYNWLDNKMHFVHLNRLHGLTIELLGRMAGFVALFDRDFQFFDEQQIADEFKLHGFVNLHSIDKKLNEVPTKKIKEIYDIRNKIIQINGNKINESVRANLIQFISSKRNYYNNAFLHVSNDEIKEKQMYDIRNHIAHFNYLTKDAADFSLIDLINELRELLHYDRKLKNAVSKAFIDLFDKHGMILKLKLNADHKLKVESLEPKKIYHLGSSAKDKPEYQYCTNQVMMAYCNMCRSLLEMKK

SEQ ID NO: 76 - protein, 1437 aa, unidentified
  source 1..1437: note = Lachnospiraceae bacterium
MKISKVREENRGAKLTVNAKTAVVSENRSQEGILYNDPSRYGKSRKNDEDRDRYIESRLKSSGKLYRIFNEDKNKRETDELQWFLSEIVKKINRRNGLVLSDMLSVDDRAFEKAFEKYAELSYTNRRNKVSGSPAFETCGVDAATAERLKGIISETNFINRIKNNIDNKVSEDIIDRIIAKYLKKSLCRERVKRGLKKLLMNAFDLPYSDPDIDVQRDFIDYVLEDFYHVRAKSQVSRSIKNMNMPVQPEGDGKFAITVSKGGTESGNKRSAEKEAFKKFLSDYASLDERVRDDMLRRMRRLVVLYFYGSDDSKLSDVNEKFDVWEDHAARRVDNREFIKLPLENKLANGKTDKDAERIRKNTVKELYRNQNIGCYRQAVKAVEEDNNGRYFDDKMLNMFFIHRIEYGVEKIYANLKQVTEFKARTGYLSEKIWKDLINYISIKYIAMGKAVYNYAMDELNASDKKEIELGKISEEYLSGISSFDYELIKAEEMLQRETAVYVAFAARHLSSQTVELDSENSDFLLLKPKGTMDKNDKNKLASNNILNFLKDKETLRDTILQYFGGHSLWTDFPFDKYLAGGKDDVDFLTDLKDVIYSMRNDSFHYATENHNNGKWNKELISAMFEHETERMTVVMKDKFYSNNLPMFYKNDDLKKLLIDLYKDNVERASQVPSFNKVFVRKNFPALVRDKDNLGIELDLKADADKGENELKFYNALYYMFKEIYYNAFLNDKNVRERFITKATKVADNYDRNKERNLKDRIKSAGSDEKKKLREQLQNYIAENDFGQRIKNIVQVNPDYTLAQICQLIMTEYNQQNNGCMQKKSAARKDINKDSYQHYKMLLLVNLRKAFLEFIKENYAFVLKPYKHDLCDKADFVPDFAKYVKPYAGLISRVAGSSELQKWYIVSRFLSPAQANHMLGFLHSYKQYVWDIYRRASETGTEINHSIAEDKIAGVDITDVDAVIDLSVKLCGTISSEISDYFKDDEVYAEYISSYLDFEYDGGNYKDSLNRFCNSDAVNDQKVALYYDGEHPKLNRNIILSKLYGERRFLEKITDRVSRSDIVEYYKLKKETSQYQTKGIFDSEDEQKNIKKFQEMKNIVEFRDLMDYSEIADELQGQLINWIYLRERDLMNFQLGYHYACLNNDSNKQATYVTLDYQGKKNRKINGAILYQICAMYINGLPLYYVDKDSSEWTVSDGKESTGAKIGEFYRYAKSFENTSDCYASGLEIFENISEHDNITELRNYIEHFRYYSSFDRSFLGIYSEVFDRFFTYDLKYRKNVPTILYNILLQHFVNVRFEFVSGKKMIGIDKKDRKIAKEKECARITIREKNGVYSEQFTYKLKNGTVYVDARDKRYLQSIIRLLFYPEKVNMDEMIEVKEKKKPSDNNTGKGYSKRDRQQDRKEYDKYKEKKKKEGNFLSGMGGNINWDEINAQLKN

SEQ ID NO: 77 - protein, 1389 aa, Leptotrichia shahii
MGNLFGHKRWYEVRDKKDFKIKRKVKVKRNYDGNKYILNINENNNKEKIDNNKFIRKYINYKKNDNILKEFTRKFHAGNILFKLKGKEGIIRIENNDDFLETEEVVLYIEAYGKSEKLKALGITKKKIIDEAIRQGITKDDKKIEIKRQENEEEIEIDIRDEYTNKTLNDCSIILRIIENDELETKKSIYEIFKNINMSLYKIIEKIIENETEKVFENRYYEEHLREKLLKDDKIDVILTNFMEIREKIKSNLEILGFVKFYLNVGGDKKKSKNKKMLVEKILNINVDLTVEDIADFVIKELEFWNITKRIEKVKKVNNEFLEKRRNRTYIKSYVLLDKHEKFKIERENKKDKIVKFFVENIKNNSIKEKIEKILAEFKIDELIKKLEKELKKGNCDTEIFGIFKKHYKVNFDSKKFSKKSDEEKELYKIIYRYLKGRIEKILVNEQKVRLKKMEKIEIEKILNESILSEKILKRVKQYTLEHIMYLGKLRHNDIDMTTVNTDDFSRLHAKEELDLELITFFASTNMELNKIFSRENINNDENIDFFGGDREKNYVLDKKILNSKIKIIRDLDFIDNKNNITNNFIRKFTKIGTNERNRILHAISKERDLQGTQDDYNKVINIIQNLKISDEEVSKALNLDVVFKDKKNIITKINDIKISEENNNDIKYLPSFSKVLPEILNLYRNNPKNEPFDTIETEKIVLNALIYVNKELYKKLILEDDLEENESKNIFLQELKKTLGNIDEIDENIIENYYKNAQISASKGNNKAIKKYQKKVIECYIGYLRKNYEELFDFSDFKMNIQEIKKQIKDINDNKTYERITVKTSDKTIVINDDFEYIISIFALLNSNAVINKIRNRFFATSVWLNTSEYQNIIDILDEIMQLNTLRNECITENWNLNLEEFIQKMKEIEKDFDDFKIQTKKEIFNNYYEDIKNNILTEFKDDINGCDVLEKKLEKIVIFDDETKFEIDKKSNILQDEQRKLSNINKKDLKKKVDQYIKDKDQEIKSKILCRIIFNSDFLKKYKKEIDNLIEDMESENENKFQEIYYPKERKNELYIYKKNLFLNIGNPNFDKIYGLISNDIKMADAKFLFNIDGKNIRKNKISEIDAILKNLNDKLNGYSKEYKEKYIKKLKENDDFFAKNIQNKNYKSFEKDYNRVSEYKKIRDLVEFNYLNKIESYLIDINWKLAIQMARFERDMHYIVNGLRELGIIKLSGYNTGISRAYPKRNGSDGFYTTTAYYKFFDEESYKKFEKICYGFGIDLSENSEINKPENESIRNYISHFYIVRNPFADYSIAEQIDRVSNLLSYSTRYNNSTYASVFEVFKKDVNLDYDELKKKFKLIGNNDILERLMKPKKVSVLELESYNSDYIKNLIIELLTKIENTNDTL

SEQ ID NO: 78 - protein, 1159 aa, Leptotrichia buccalis; identical to SEQ ID NO: 73

SEQ ID NO: 79 - protein, 1127 aa, Prevotella buccae
MQKQDKLFVDRKKNAIFAFPKYITIMENKEKPEPIYYELTDKHFWAAFLNLARHNVYTTINHINRRLEIAELKDDGYMMGIKGSWNEQAKKLDKKVRLRDLIMKHFPFLEAAAYEMTNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYNAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSAHNNLFPSLQTDKTCNGRSKFNLGTQFTAEAFLSVHELLPMMFYYLLLTKDYSRKESADKVEGIIRKEISNIYAIYDAFANNEINSIADLTRRLQNTNILQGHLPKQMISILKGRQKDMGKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNRLKPKKRQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKALVKDRRLNGLFSFAETTDLNLEEHPISKLSVDLELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 80 - protein, 1127 aa, Prevotella buccae
MQKQDKLFVDRKKNAIFAFPKYITIMENQEKPEPIYYELTDKHFWAAFLNLARHNVYTTINHINRRLEIAELKDDGYMMDIKGSWNEQAKKLDKKVRLRDLIMKHFPFLEAAAYEITNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYDAEEEPFKNTLVRHQDRFPYFVLRYFDLNEIFEQLRFQIDLGTYHFSIYNKRIGDEDEVRHLTHHLYGFARIQDFAQQNQPEVWRKLVKDLDYFEASQEPYIPKTAPHYHLENEKIGIKFCSTHNNLFPSLKTEKTCNGRSKFNLGTQFTAEAFLSVHELLPMMFYYLLLTKDYSRKESADKVEGIIRKEISNIYAIYDAFANGEINSIADLTCRLQKTNILQGHLPKQMISILEGRQKDMEKEAERKIGEMIDDTQRRLDLLCKQTNQKIRIGKRNAGLLKSGKIADWLVNDMMRFQPVQKDQNNIPINNSKANSTEYRMLQRALALFGSENFRLKAYFNQMNLVGNDNPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNKLKPQKGQFLDKKERVELWQKNKELFKNYPSEKKKTDLAYLDFLSWKKFERELRLIKNQDIVTWLMFKELFNMATVEGLKIGEIHLRDIDTNTANEESNNILNRIMPMKLPVKTYETDNKGNILKERPLATFYIEETETKVLKQGNFKVLAKDRRLNGLLSFAETTDIDLEKNPITKLSVDHELIKYQTTRISIFEMTLGLEKKLINKYPTLPTDSFRNMLERWLQCKANRPELKNYVNSLIAVRNAFSHNQYPMYDATLFAEVKKFTLFPSVDTKKIELNIAPQLLEIVGKAIKEIEKSENKN

SEQ ID NO: 81 - protein, 1232 aa, Bergeyella zoohelcum
XENKTSLGNNIYYNPFKPQDKSYFAGYFNAAXENTDSVFRELGKRLKGKEYTSENFFDAIFKENISLVEYERYVKLLSDYFPXARLLDKKEVPIKERKENFKKNFKGIIKAVRDLRNFYTHKEHGEVEITDEIFGVLDEXLKSTVLTVKKKKVKTDKTKEILKKSIEKQLDILCQKKLEYLRDTARKIEEKRRNQRERGEKELVAPFKYSDKRDDLIAAIYNDAFDVYIDKKKDSLKESSKAKYNTKSDPQQEEGDLKIPISKNGVVFLLSLFLTKQEIHAFKSKIAGFKATVIDEATVSEATVSHGKNSICFXATHEIFSHLAYKKLKRKVRTAEINYGEAENAEQLSVYAKETLXXQXLDELSKVPDVVYQNLSEDVQKTFIEDWNEYLKENNGDVGTXEEEQVIHPVIRKRYEDKFNYFAIRFLDEFAQFPTLRFQVHLGNYLHDSRPKENLISDRRIKEKITVFGRLSELEHKKALFIKNTETNEDREHYWEIFPNPNYDFPKENISVNDKDFPIAGSILDREKQPVAGKIGIKVKLLNQQYVSEVDKAVKAHQLKQRKASKPSIQNIIEEIVPINESNPKEAIVFGGQPTAYLSXNDIHSILYEFFDKWEKKKEKLEKKGEKELRKEIGKELEKKIVGKIQAQIQQIIDKDTNAKILKPYQDGNSTAIDKEKLIKDLKQEQNILQKLKDEQTVREKEYNDFIAYQDKNREINKVRDRNHKQYLKDNLKRKYPEAPARKEVLYYREKGKVAVWLANDIKRFXPTDFKNEWKGEQHSLLQKSLAYYEQCKEELKNLLPEKVFQHLPFKLGGYFQQKYLYQFYTCYLDKRLEYISGLVQQAENFKSENKVFKKVENECFKFLKKQNYTHKELDARVQSILGYPIFLERGFXDEKPTIIKGKTFKGNEALFADWFRYYKEYQNFQTFYDTENYPLVELEKKQADRKRKTKIYQQKKNDVFTLLXAKHIFKSVFKQDSIDQFSLEDLYQSREERLGNQERARQTGERNTNYIWNKTVDLKLCDGKITVENVKLKNVGDFIKYEYDQRVQAFLKYEENIEWQAFLIKESKEEENYPYVVEREIEQYEKVRREELLKEVHLIEEYILEKVKDKEILKKGDNQNFKYYILNGLLKQLKNEDVESYKVFNLNTEPEDVNINQLKQEATDLEQKAFVLTYIANKFAHNQLPKKEFWDYCQEKYGKIEKEKTYAEYFAEVFKKEKEALIKLEHHHHHH

SEQ ID NO: 82 - protein, 1127 aa, Prevotella sp. MSX73
MQKQDKLFVDRKKNAIFAFPKYITIMENQEKPEPIYYELTDKHFWAAFLNLARHNVYTTINHINRRLEIAELKDDGYMMGIKGSWNEQAKKLDKKVRLRDLIMKHFPFLEAAAYEITNSKSPNNKEQREKEQSEALSLNNLKNVLFIFLEKLQVLRNYYSHYKYSEESPKPIFETSLLKNMYKVFDANVRLVKRDYMHHENIDMQRDFTHLNRKKQVGRTKNIIDSPNFHYHFADKEGNMTIAGLLFFVSLFLDKKDAIWMQKKLKGFKDGRNLREQMTNEVFCRSRISLPKLKLENVQTKDWMQLDMLNELVRCPKSLYERLREKDRESFKVPFDIFSDDYDAEEEPFKNTLVRHQDRFPYFVLRYFDLNEIFEQLRFQIDLGTYHFSIYNKRIGDEDEVRHLTHHLYGFARIQDFAPQNQPEEWRKLVKDLDHFETSQEPYISKTAPHYHLENEKIGIKFCSTHNNLFPSLKREKTCNGRSKFNLGTQFTAEAFLSVHELLPMMFYYLLLTKDYSRKESADKVEGIIRKEISNIYAIYDAFANNEINSIADLTCRLQKTNILQGHLPKQMISILEGRQKDMEKEAERKIGEMIDDTQRRLDLLCKQTNQKIRIGKRNAGLLKSGKIADWLVSDMMRFQPVQKDTNNAPINNSKANSTEYRMLQHALALFGSESSRLKAYFRQMNLVGNANPHPFLAETQWEHQTNILSFYRNYLEARKKYLKGLKPQNWKQYQHFLILKVQKTNRNTLVTGWKNSFNLPRGIFTQPIREWFEKHNNSKRIYDQILSFDRVGFVAKAIPLYFAEEYKDNVQPFYDYPFNIGNKLKPQKGQFLDKKERVELWQKNKELFKNYPSEKNKTDLAYLDFLSWKKFERELRLIKNQDIVTWLMFKELFKTTTVEGLKIGEIHLRDIDTNTANEESNNILNRIMPMKLPVKTYETDNKGNILKERPLATFYIEETETKVLKQGNFKVLAKDRRLNGLLSFAETTDIDLEKNPITKLSVDYELIKYQTTRISIFEMTLGLEKKLIDKYSTLPTDSFRNMLERWLQCKANRPELKNYVNSLIAVRNAFSHNQYPMYDATLFAEVKKFTLFPSVDTKKIELNIAPQLLEIVGKAIKEIEKSENKN

(19) United States
(12) Patent Application Publication
(10) Pub. No.: US 2024/0309473 A1
(43) Pub. Date: Sep. 19, 2024

(54) KINETIC BARCODING TO ENHANCE SPECIFICITY OF CRISPR/CAS REACTIONS

(71) Applicants: THE J. DAVID GLADSTONE INSTITUTES, A TESTAMENTARY TRUST ESTABLISHED UNDER THE WELL OF J., San Francisco, CA (US); REGENTS OF THE UNIVERSITY OF CALIFORNIA, Oakland, CA (US)

(72) Inventors: Daniel Fletcher, Oakland, CA (US); Sungmin Son, Oakland, CA (US); Melanie Ott, Mill Valley, CA (US)

(21) Appl. No.: 18/575,944

(22) PCT Filed: Jul. 1, 2022
(86) PCT No.: PCT/US2022/035938

§ 371 Date: Jan. 2, 2024

Related U.S. Application Data
(60) Provisional application No. 63/217,836, filed on Jul. 2, 2021.

(51) Int. Cl. C12Q 1/70; C12Q 1/6832

(52) U.S. Cl. CPC C12Q 1/701; C12Q 1/6832

(57) ABSTRACT

Droplet detection of RNA enables quantification of the absolute amount of target RNA as well as identification of different variant or mutant RNA species. As described herein. RNA detection with high sensitivity and multiplexed specificity can be achieved with short detection times by encapsulating the Cas reaction in droplets and monitoring enzyme kinetics fluorescently.

### CROSS REFERENCE TO RELATED APPLICATIONS

This application is a U.S. National Stage Filing under 35 U.S.C. 371 from International Patent Application Serial No. PCT/US2022/035938 filed Jul. 1, 2022, Published as WO2023/278834 on Jan. 5, 2023, which application claims the benefit of priority to U.S. Provisional Patent Application Ser. No. 63/217,836, filed Jul. 2, 2021, the complete disclosures of which are incorporated herein by reference in their entireties.

### GOVERNMENT FUNDING

This invention was made with government support under US4 HL143541 awarded by the National Institutes of Health. The government has certain rights in the invention.

### INCORPORATION BY REFERENCE OF SEQUENCE LISTING

This application contains a Sequence Listing which has been submitted electronically in ST26 format and is hereby incorporated by reference in its entirety. Said ST26 file, created on Feb. 5, 2024, is named “3730196US1.xml” and is 89,825 bytes in size.

### BACKGROUND

The PCR-based assays are currently the gold standard for RNA detection, as they can achieve high sensitivity (˜1 copy/μL) with assay times under 2 hours. CRISPR-Cas13, a type VI CRISPR system, offers an alternate way of quantifying RNA by using its RNA-activated RNase activity to cleave a fluorescent reporter upon guide RNA-directed binding of a target RNA (East-Seletsky et al., 2016). Though Cas13 can be combined with reverse transcription, amplification, and transcription to increase sensitivity (Gootenberg et al., 2017), direct detection of RNA with Cas13 avoids the limitations of those steps and can achieve modest sensitivity by combining multiple crRNAs recognizing different regions of the target RNA. For the SAR-CoV-2 genome, direct detection with LbuCas13a was able to measure as little as about 200 copies/μL in 30 minutes while employing three crRNAs (Fozouni et al., 2021) and about 63 copies/μL in 2 hours while employing eight crRNAs (Liu et al., 2021). However, PCR-level sensitivity has not yet been achieved with direct Cas13 detection, and approaches for identifying which of multiple virus variants are present in a single sample are limited (Jiao et al., 2021).

Current uses of Cas13 and Cas12 nucleases for diagnostic applications also rely on bulk reactions that produce fluorescent signals in the presence of target RNA or DNA, which means the kinetics of individual Cas-guide-target complexes cannot be observed. Only the bulk combination of Cas-guide-target complex signals can be observed by currently available methods. As a result, such bulk assay methods are not suitable for detecting variants.

### SUMMARY

As described herein, RNA detection with high sensitivity and multiplexed specificity can be achieved with short detection times by encapsulating the Cas nuclease reaction in droplets and measuring/monitoring the kinetics of the Cas nuclease reaction. Droplet detection of RNA targets enables quantification of the absolute amount of each target RNA based on the number of positive droplets. Unlike droplet digital PCR (ddPCR), the small droplet volumes used in the methods described herein accelerate signal accumulation of the direct Cas nuclease reaction. For example, when a single target RNA is encapsulated in a droplet with a volume of about 10 picoliters, the Cas13 signal accumulation rate is equivalent to that of a bulk reaction containing 10⁵ copies/μL of target RNA (see, e.g., FIG. 1A). Moreover, different target RNAs (e.g., different RNA viruses) can simultaneously be detected by using different CRISPR guide RNAs (crRNAs).

Described herein are assay mixtures that include a population of droplets ranging in diameter from at least 10 to 60 μm, the population comprising a test droplet subpopulation comprising at least one ribonucleoprotein complex, plus at least one reporter RNA, plus at least one target RNA. The ribonucleoprotein complex can include a Cas nuclease and a CRISPR guide RNA (crRNA). Upon binding of the ribonucleoprotein complex (via the crRNA), the ribonucleoprotein complex cleaves Reporter RNAs, to release a detectable signal. Assay mixtures are therefore described herein that can include a population of droplets. The mean diameter of the droplets can range from at least 10 to 60 μm. The droplet population including a test droplet subpopulation that includes at least one ribonucleoprotein (RNP) complex, plus at least one reporter RNA, plus at least one target RNA. In some cases, the population can include droplets that do not include one or more of a ribonucleoprotein complex, a reporter RNA, or a target RNA; these droplets can be used as control droplets. For example, the control droplets can be used to define background levels of fluorescence.

In some cases the crRNA(s) can have a polymer covalently linked to the crRNA 5′ end. Ribonucleoprotein complexes of a cas nuclease and such a crRNA-polymer hybrid exhibit reduced nuclease activity, which can facilitate analysis of the kinetics of the nuclease reaction. In addition, use of different polymers on different crRNAs can enhance differences in signal kinetics, thereby improving detection of different target RNAs in a complex mixture of target RNAs. In some cases, bulk assays that include a series of different crRNA-polymer hybrids (and at least one type of cas nuclease) can provide readily distinguishable signals from different target RNA interactions. Hence, use of droplet assays are not allows needed when using crRNA-polymer hybrids to detect and identify different target RNAs.

The polymer used for crRNA-polymer hybrid can, for example, be polyethylene glycol, poly[oxy(11-(3-(9-adeninyl)propionato)-undecanyl-1-thiomethyl)ethylene] (PECH-AP), poly[oxy(11-(5-(9-adenylethyloxy)-4-oxopentanoato)undecanyl-1-thiomethyl)ethylene] (PECH-AS), DNA, or a combination thereof. In some cases, the polymer is DNA (either single-stranded or double-stranded DNA). It is thought that the polymers used for crRNA-polymer hybrids can reduce folding, formation, or activity of a higher-eukaryotes-and-prokaryotes nucleotide-binding (HEPN) domain of a Cas nuclease in a ribonucleoprotein complex with the crRNA-polymer hybrid. For example, the polymer may at least partially block the HEPN domain. The polymer can be of variable length, but in general, longer polymers reduce the nuclease activities of ribonucleoprotein complexes to a greater extent than shorter polymers.

Also described herein are methods for detecting and/or identifying at least one target RNA. Such methods can involve measuring and/or monitoring fluorescence of individual droplets in a population of droplets, where the population includes at least one droplet comprising a target RNA, a ribonucleoprotein complex, and at least one type of reporter RNA. Such a droplet population comprises at least two, at least three, at least five, at least seven, at least ten, at least fifteen, at least twenty, at least thirty, at least fifty droplets, each comprising a target RNA, a ribonucleoprotein complex, and at least one type of reporter RNA.

Also described herein are methods for detecting and/or identifying at least one target RNA that involve use of crRNA-polymer hybrids. Such methods can involve measuring and/or monitoring fluorescence of an assay mixture that can include at least one target RNA, a ribonucleoprotein complex, and at least one type of reporter RNA, where the ribonucleoprotein complex includes a cas nuclease and a crRNA-polymer hybrid.

The target RNA can be the same target RNA throughout the population of droplets. However, in many cases different droplets can each contain a different target RNA or a different combination of target RNAs. The target RNAs can be one or more viral RNAs, prokaryotic RNAs, eukaryotic RNAs, or combinations thereof.

In some cases, at least one target RNA can be a wild type target RNA sequence. In some cases, at least one target RNA can be a variant or mutant target RNA sequence.

Examples of target RNAs includes RNAs from one or more SARS coronaviruses (SARS-CoV-1 and/or SARS-CoV-2), Orthomyxoviruses (influenza viruses), Hepatitis C Viruses (HCVs), Ebola, influenza viruses, polio viruses, measles viruses, retroviruses, Human T-cell lymphotropic virus type 1 (HTLV-1), human immunodeficiency viruses (HIV), or combinations thereof. In some cases, at least one target RNA is a coronavirus RNA. In some cases, at least one target RNA can be an RNA for a disease marker. In some cases, at least one target RNA can be a microRNA.

Also described herein are methods that can involve (a) contacting a sample with at least one type of ribonucleoprotein complex and at least one type of reporter RNA to form a reaction mixture; (b) mixing the reaction mixture with oil and surfactant to form an emulsion comprising water-in-oil droplets, where at least some of the droplets encapsulate all components of the reaction mixture; (c) removing excess oil from the droplets; (d) selecting at least 1 droplet, or at least 3 droplets, or at least 10 droplets that emit fluorescence as positive droplets for monitoring; and (e) monitoring the fluorescence of the positive droplets over time.

The assay mixtures and methods described herein can be used to detect and/or identify various RNA viruses in samples from a variety of sources. For example, samples can be environmental samples (water, sewage, soil, waste, manure, liquids, or combinations thereof) or samples from one or more animals. The animals can be one or more human(s), birds, mammals, domesticated animals, zoo animals, wild animals, or combinations thereof. The samples from animals can include bodily fluids, excretions, tissues, or combinations thereof. The assay mixtures and methods described herein can distinguish between wild type RNAs, mutant RNAs, and variant RNAs.

### DESCRIPTION OF THE DRAWINGS

FIG. 1A-1N illustrate rapid detection of target RNA molecule Cas reactions within heterogeneous droplets. FIG. 1A is a schematic illustrating an increased signal accumulation rate for a single Cas13 confined in decreasing volumes (red: activated Cas13a, white: inactive Cas13a). Each droplet contains hundreds of thousand copies of Cas13a RNP and millions of quenched RNA reporter. Only the droplet possessing one or more target RNA will acquire signal. FIG. 1B is a schematic illustrating a droplet Cas13a assay method. A Cas13a reaction including one or more guide RNAs and target RNAs are mixed with an oil (HFE 7500 including 2 wt % Perfluoro-PEG surfactant) and emulsified by repeated pipette mixing at a constant speed for 2 minutes. The emulsified reaction is typically incubated for 15 minutes in 37° C. and the reaction is optionally quenched on ice. Subsequently, the emulsion is loaded into a custom flow cell and imaged with a fluorescent microscope. The complete assay takes 20 minutes. FIG. 1C graphically illustrates the size distribution of droplets, which is reduced by 0.1 wt % IGEPAL in the Cas13a mix. IGEPAL is a nonionic, non-denaturing detergent. The top (red) line indicates the mean distribution of droplet size in the presence of 0.1 vol % IGEPAL. The lower (black) darker shaded line indicates droplets in the absence of IGEPAL. The shadows indicate the S.D. from 5 independent droplet preparations. FIG. 1D shows bright field (left) and fluorescent images of Cas13a reaction taken with a 20× objective lens. Time (T)=0, 10, and 20 minutes since the beginning of imaging. Scale bar=65 μm. FIG. 1E graphically illustrate the fluorescent signal over time in three positive droplets (the top three lines) and one background droplet (the bottom line). The signal is the mean fluorescent intensity change within a droplet normalized by the initial signal after background subtraction. Images are acquired every 30 seconds and corrected for the photobleaching (see Example 1). FIG. 1F graphically illustrates the single Cas13a turnover frequency measured from individual droplets containing crRNA 4 (SEQ ID NO:4) and SARS-CoV-2 RNA (N=478 droplets). Guide RNA crRNA 4 (SEQ ID NO:4) targets the N gene of SARS-CoV-2 RNA. The box and whisker plot in the right panel indicates the median, the lower and upper quartiles, and the minimum and the maximum values. FIG. 1G graphically illustrates the signal-per-droplet with increasing incubation times is represented as the box and whisker plot. The signal is normalized by the median in 5 minutes timepoint. N>800 droplets are used in all four timepoints. FIG. 1H graphically illustrates the number of positive droplets detected with increasing incubation times. 1×10⁴ copies/μL of SARS-CoV-2 RNA were added to a bulk reaction prior to droplet formation and droplets are incubated for a specified time. Data are represented as mean ±SD of three technical replicates. P-values are determined from a two-tailed Student's t-test: ns=not significant. FIG. 1I shows an image of an automatic multi-channel pipettor (an 8-channel pipette; Integra biosciences, Part #4623), which was used to generate emulsions. Simples of about 110 μL were mixed with the pipettor for 150 repetitions at the maximum speed (speed 10) to emulsify droplets to a narrow size range. The emulsion so formed was either directly loaded into a flow cell for time course imaging or incubated in a heating block at 37° C. before being transferred and imaged. FIG. 1J is a schematic illustrating confocal imaging of a droplet at its midplane. FIG. 1K graphically illustrates the reaction velocities (change of cleaved reporter) in differently sized droplets. FIG. 1L graphically illustrates the turnover frequency, as measured by total change of cleaved reporter in droplets with different diameters. FIG. 1M graphically illustrates identification of positive reactions in droplet assays in reaction times of 15 minutes using a 4×/0.20NA microscope objective. FIG. 1N graphically illustrates identification of positive reactions in droplet assays at various reaction time, where S/B refers to signal over background.

FIG. 2A-2H illustrate the detection sensitivity of droplet Cas13a assay using crRNA combinations. FIG. 2A is a schematic illustrating two potential results of Cas13 droplet reactions that use two different crRNAs simultaneously: (1) Complete loading-if the whole N gene segment is loaded to one droplet containing crRNAs targeting two different regions of N gene, the signal will accumulate twice as fast as a droplet containing one copy of target RNA; or (2) Fragmented loading-if one N gene is fragmented to two halves and loaded into two separate droplets, the number of positive droplet will be doubled while the signal of individual ones remain identical to the droplet containing one copy of target RNA. FIG. 2B graphically illustrates the distribution of signal-per-droplet for a Cas13a reaction as shown in a box and whisker plot. N>250 for all three conditions. The Cas13a reaction included 2.5×10⁴ copies/μL of in vitro transcribed (IVT) N gene in a droplet assay mixture. A control Cas13a assay included no target RNA. The droplet assays included the following guide RNAs: only crRNA2 (SEQ ID NO:2), only crRNA 4 (SEQ ID NO:4), or both crRNA2 and crRNA4. Droplets were quantified after 1 hour of reaction incubation FIG. 2C graphically illustrates the data for the assay described for FIG. 2B as number of positive droplets per mm² (mean±SD of three replicates). FIG. 2D graphically illustrates the number of positive droplets quantified for different crRNA combinations after adding 100 copies/μL. of externally quantified SARS-CoV-2 RNA (BEI resources). Each reaction was incubated for 15 minutes and droplet images were taken with the 4× objective lens. Data are represented as mean±SD of three replicates. FIG. 2E graphically illustrates the number of positive droplets quantified for a series of dilutions of externally quantified SARS-CoV-2 RNA. Each reaction was incubated for 15 minutes. Data are represented as mean±SD of three replicates. P-values were determined based on a two-tailed Student's t-test: ns=not significant, *p<0.05, ** p<0.005, *** p<0.001. FIG. 2F graphically illustrates the signal from individual assays using either crRNA 2 (SEQ ID NO:2, to line) or crRNA 4 (SEQ ID NO:4; lower line). FIG. 2G graphically illustrates that the activity of Cas13a remains constant even when only a small fraction of total RNPs in a droplet contained crRNA matching the target. FIG. 2H graphically illustrates that the limit of detection was not improved when the assay reaction was incubated for 30 minutes instead of 15 minutes. SARS-CoV-2 RNA was used as the target.

FIG. 3A-3Q illustrate crRNA-dependent heterogenous Cas13a activities. FIG. 3A graphically illustrates the slope of a bulk Cas13a reaction containing 3.5×10⁴ copies/μL of SARS-CoV-2 RNAs using crRNAs that target different regions of the N gene (crRNA 4, crRNA11A, crRNA12A). A control assay had no target RNA. The slope was determined by performing simple linear regression of data from each replicate (N=3) individually. Data are represented as mean±SD. FIG. 3B graphically illustrates the number of positive droplets for a droplet Cas13a reaction with 3.5×10+copies/μL of SARS-CoV-2 RNA after 30 minutes of incubation. A control RNP only assay had no SARS-CoV-2 RNA. Data are represented as mean±SD of three replicates. For the RNP only condition, one measurement for each crRNA is merged. The crRNAs employed were crRNA 4, crRNA11A, crRNA12A. FIG. 3C graphically illustrates the signal per droplet when different crRNAs were used in the droplet Cas13a reaction with 3.5×10⁴ copies/μL of SARS-CoV-2 RNA described for FIG. 3B. Data are represented as box and whisker plots marking the median, the lower and upper quartiles, and the minimum and the maximum values. The signals were normalized by the median of crRNA 4. The crRNAs employed were crRNA 4, crRNA1LA, crRNA12A. N>1800 droplets were used in all three conditions. FIG. 3D graphically illustrates signal trajectories over time for droplet assays for detecting SARS-CoV-2 RNA using crRNA 4 (SEQ ID NO:4). FIG. 3E graphically illustrates signal trajectories over time for droplet assays for detecting SARS-CoV-2 RNA using crRNA 11A (SEQ ID NO:36). FIG. 3F graphically illustrates signal trajectories over time for droplet assays for detecting SARS-CoV-2 RNA using crRNA 12A (SEQ ID NO:37). For FIG. 3D-3F the one hundred individual trajectories were monitored from droplets ranging from 30 to 36 μm size (show as the grey lines) along with arbitrarily selected, representative trajectories (the red lines). Signals were measured every 30 seconds for each trajectory. Data from two replicate runs were combined for each crRNA. FIG. 3G illustrates time trajectories of the rare positive droplets from the Cas13a reactions without any target RNA. Thirty-one individual trajectories were measured in droplets ranging from 30 to 36 μm size, from two replicate runs. FIG. 3H graphically illustrates the slope over time for droplet assays, illustrating the analytical strategy for individual Cas13a signal trajectories. The darker line is an example trajectory obtained with crRNA 12A. The average slope (Slope (avg)), time from target addition to the initiation of enzyme activity (Tinit), and the Root-mean-square-deviation (RMSD) are determined by performing simple linear regression to the raw signal. Slopefast and Slopeslow correspond to the fast and slow periods of signal emission as illustrated here and as determined as shown in FIG. 3I. FIG. 3I illustrates the calculation of the instantaneous slopes by taking the time-derivative of the raw signal (the shaded histogram) and its probability distribution as fitted with either a single-distribution or via binary-gaussian distributions (the line). For data that favors the binary distribution, the Slopefast, Slopeslow, % Fast, and % Slow were determined from the mean and the proportion of each gaussian peak. FIG. 3J graphically illustrates the normalized slope of droplet assay signals for different crRNA represented as the box and whisker plots including outliers. The crRNAs employed were crRNA 4, crRNA11A, crRNA12A. FIG. 3K graphically illustrates the percentage of “fast” slope droplets for assays using different crRNAs. The crRNAs employed were crRNA 4, crRNA11A, crRNA12A. FIG. 3L graphically illustrates the root-mean-square-deviation (RMSD) of signals from droplet assays using different crRNAs. FIG. 3M graphically illustrates the time from target addition to the initiation of enzyme activity (Tinit) for droplet assays using different crRNAs. The crRNAs employed were crRNA 4, crRNA11A, crRNA12A. For FIGS. 3J-3M the distributions of key Cas13a kinetic parameters are represented as the box and whisker plots including outliers. Individual 30-minutes-long trajectories from droplets of arbitrary size is used after their signal is normalized for droplet size. N>250 for all conditions. P-values are determined from a two-tailed Student's t-test: ns=not significant, *p<0.05, *** p<0.001. FIG. 3N graphically illustrates the average slope (Slope (avg)), time from target addition to the initiation of enzyme activity (Tinit), and the Root-mean-square-deviation (RMSD) for droplet assays using crRNA 4 (SEQ ID NO:4) at low concentrations with SARS-CoV-2 RNA. FIG. 3O graphically illustrates average slope (Slope (avg)), time from target addition to the initiation of enzyme activity (Tinit), and the Root-mean-square-deviation (RMSD) for droplet assays using crRNA 12 (SEQ ID NO:12) at high concentrations. FIG. 3P is a schematic illustrating recognition of target by RNP containing a Cas nuclease and a guide crRNA that upon binding the target activates the nuclease to cleave a reporter RNA, which generates the signal during the droplet assay. The two graphs illustrate signal trajectories over time for crRNA 4 (middle) and crRNA 12 (right) droplet assays. FIG. 3Q graphically illustrates average slope (Slope (avg)), time from target addition to the initiation of enzyme activity (Tinit), and the Root-mean-square-deviation (RMSD) for droplet assays using crRNA 2 (SEQ ID NO:2) and a full length SARS-CoV-2 RNA target.

FIG. 4A-4N illustrate the kinetic-barcoding methods for multiplexed detection of virus. FIG. 4A is a schematic diagram illustrating the kinetic-barcoding method for simultaneous detection of two different viruses. FIG. 4B is a schematic diagram illustrating the kinetic-barcoding method for simultaneous detection of two different variants. The kinetic-barcoding method detects unique Cas13a kinetic signatures for specific combinations of crRNA guides and target RNAs. FIG. 4C shows representative graphs illustrating single Cas13a reaction trajectories when human coronavirus strain NL 63 (HCoV-NL 63) RNA was targeted by crRNA 7 or when SARS-CoV-2 RNA was targeted by crRNA 12. The signals were total fluorescence change in a droplet, which remains invariant regardless of droplet size. The dotted red line shows a linear fit. FIG. 4D graphically illustrates the distribution between HCOV and SARS-CoV-2 of slope and RMSD values for individual Cas13a signal trajectories in droplet assays The slope and RMSD values were determined from individual 30 minutes-long trajectories (N=488). The RMSD values were first normalized by the mean signal of the same trajectory and then normalized to 0 to 1. The slope values were normalized to 0 to 1. FIG. 4E graphically illustrates identification of HCoV or SARS-CoV-2 based on the kinetic parameters of individual Cas13 reactions. Varying numbers of 30-minutes-long Cas13a trajectories were randomly selected from each condition and the difference between two groups was quantified as p-values based on a two-tailed Student's t-test.

FIG. 4F shows representative graphs illustrating single Cas13a reaction trajectories using an RNA target that included the wild type SARS-CoV-2 S gene or an RNA target that included the D614G mutation in the SARS-CoV-2 S gene. FIG. 4G graphically illustrates the distribution between slope values and RMSD values of individual Cas13a signal trajectories of targets having either the wild type SARS-CoV-2 S gene or the

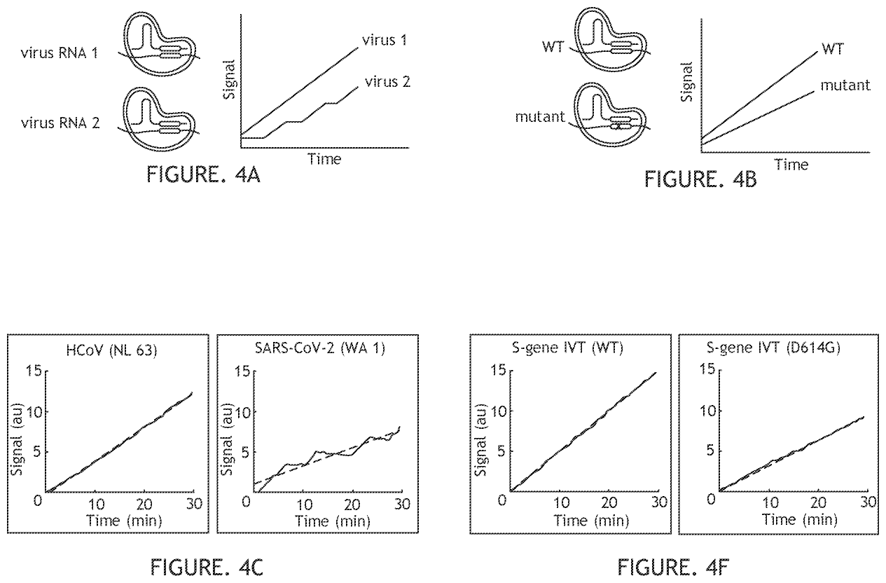

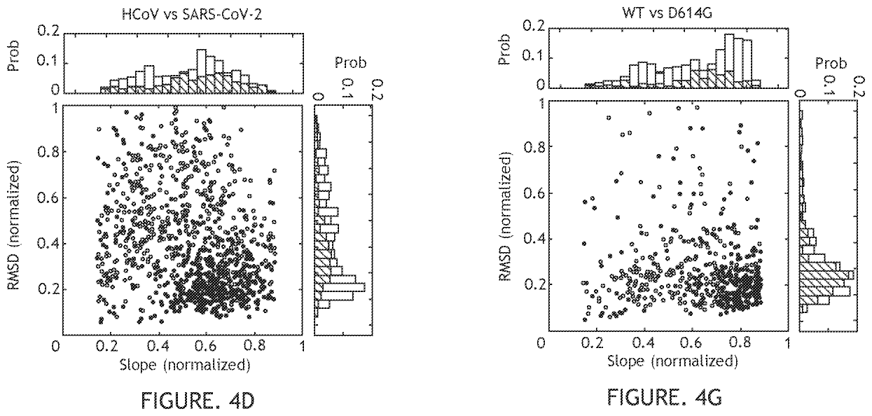

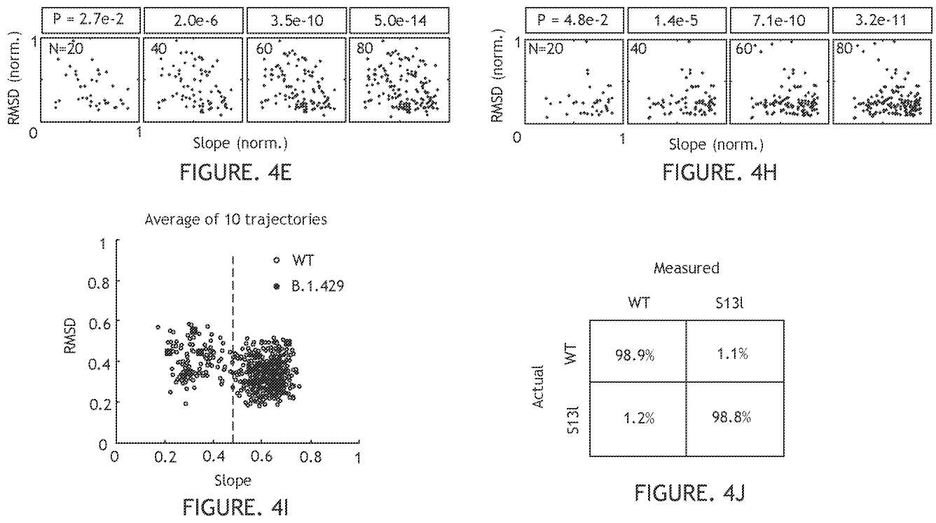

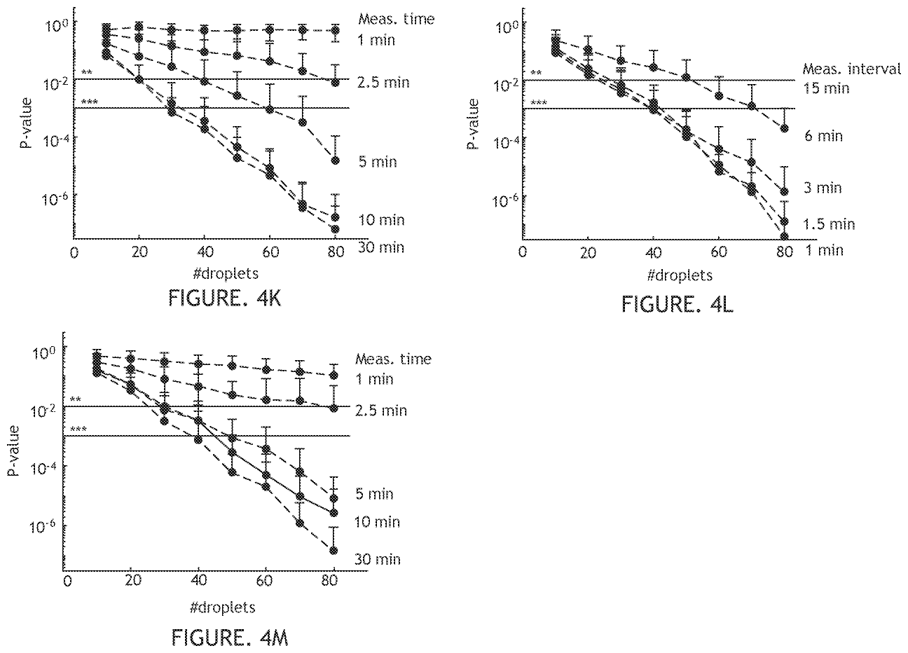

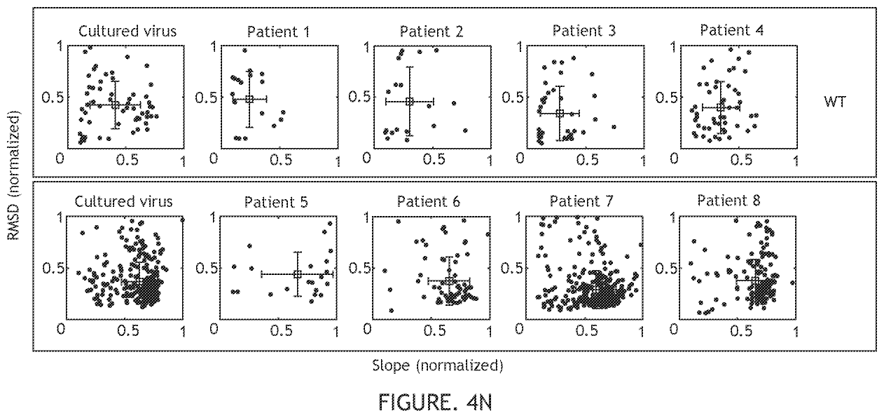

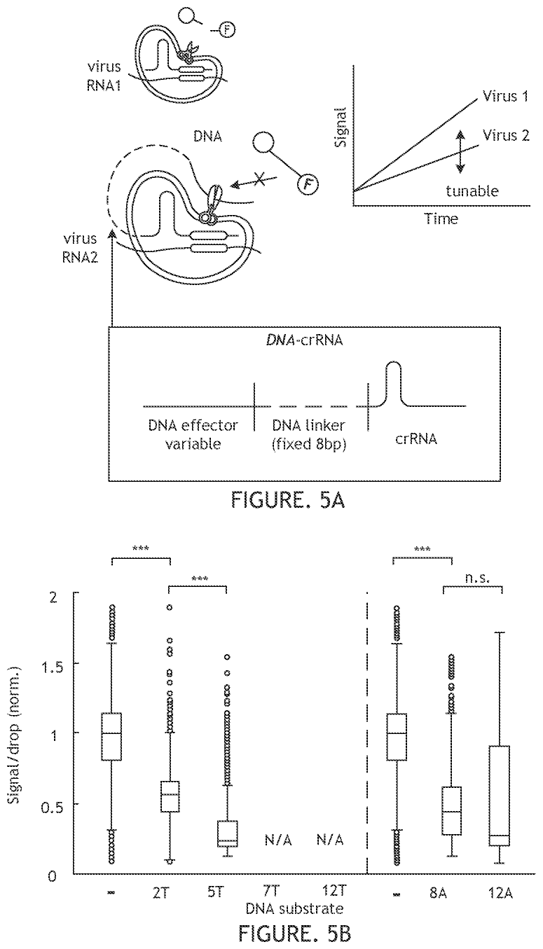

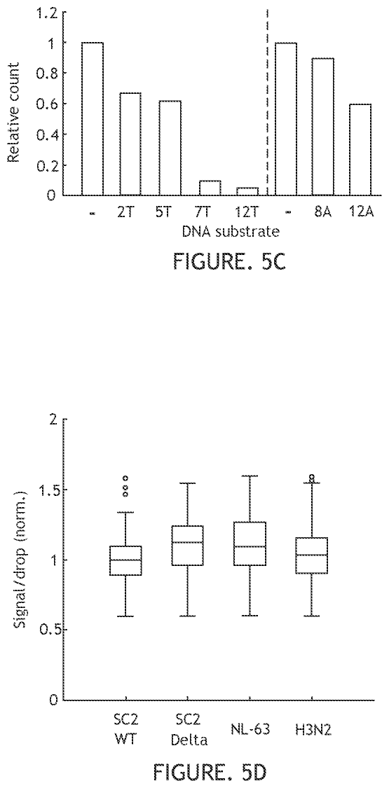

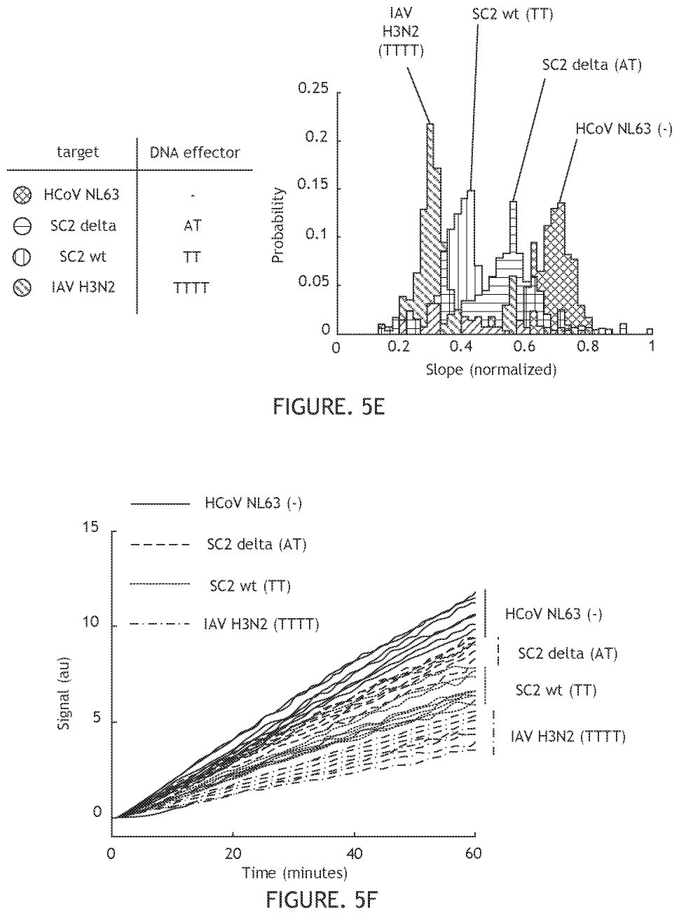

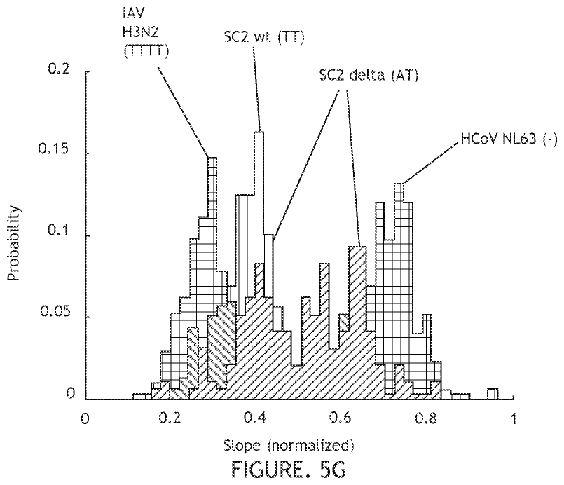

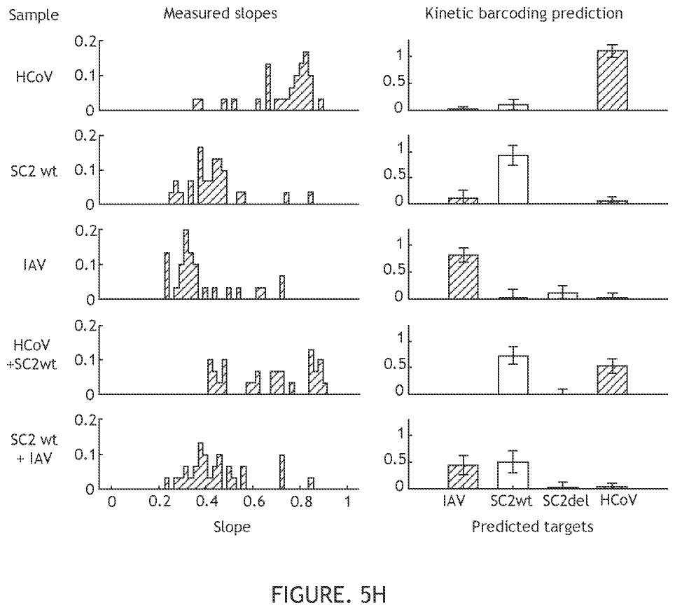

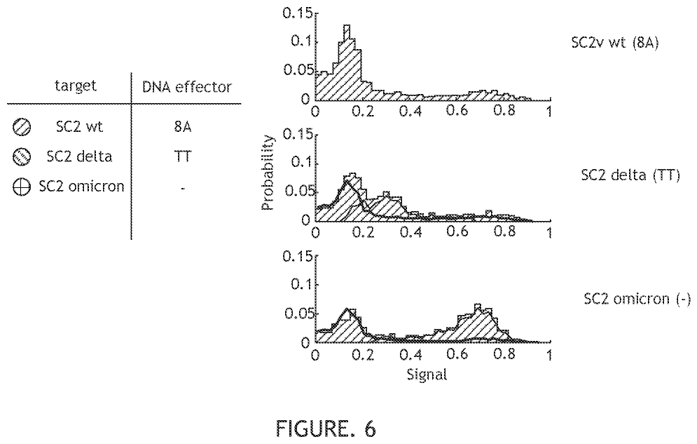

D614G mutant SARS-CoV-2 S gene (N=208). FIG. 4H graphically illustrates identification of wild type SARS-CoV-2 (signals more to the left) or the D614G mutant SARS-CoV-2 strain based on the kinetic parameters of individual Cas13 reactions. Varying numbers of 30-minutes-long Cas13a trajectories were randomly selected from each condition and the difference between two groups was quantified as p-values based on a two-tailed Student's t-test. FIG. 41 graphically illustrates identification of the

SARS-CoV-2 B.1.427 variant (signals more to the right) from clinical samples using the kinetic-barcoding methods. The average of slope or RMSD distribution was obtained by randomly selecting ten positive trajectories from many trajectories measured for each sample. In the original, the blue dots are WT (N=26) and cluster more to the left, while the magenta dots in the original are B.1.427 (N=86) and cluster to the right. The squares are example values for WT on the left and the SARS-CoV-2 B.1.427 variant on the right. The black dotted line indicates the slope threshold separating the WT from B.1.427 data. FIG. 4J graphically illustrates the detection specificity of kinetic barcoding. The accuracy was determined from FIG. 4I. FIG. 4K graphically illustrates the p-values of increasing numbers of signal trajectories over time. The measurement interval was 30 seconds. Although extending the measurement time improved classification, measurement times longer than 10 minutes did not provide any improvement. FIG. 4L graphically illustrates the p-values of increasing numbers of signal trajectories over time, where images were acquired every 3 minutes for 30 minutes instead of every 30 seconds for 10 minutes as shown for FIG. 4K. The total measurement time was 30 minutes. FIG. 4M graphically illustrates the p-values of increasing numbers of signal trajectories for the SARS-CoV-2 D614G mutant RNA over time, illustrating the difference in the average slopes of 30 or more signal trajectories. The measurement interval was 30 seconds. These data illustrate that the D614G mutant RNA could be distinguished from the wild type RNA within 5 minutes. FIG. 4N graphically illustrates RMSD vs slope values for a series of patient samples previously shown to be infected with SARS-CoV-2 (i.e., exhibiting Ct values of 15 to 20 in PCR testing). Positive trajectories (N=15 to 350) were measured among the droplets in the assays. Although individual trajectories from each sample exhibited heterogenous slopes and RMSDs, the slopes measured from the WT were significantly lower than those measured from the B.1.427 mutant (FIGS. 4I and 4N).

FIG. 5A-5H illustrate modulation of Cas13a nuclease activity when a DNA fragment of varying sequence and length is added to the 5′-end of crRNA to form a DNA-crRNA, so that the DNA extension can interfere with the Cas nuclease HEPN site when the crRNA is loaded. FIG. 5A is a schematic diagram illustrating the structure of a ribonucleoprotein complex between a Cas nuclease and a crRNA, where the crRNA can have a DNA fragment of varying sequence and length linked to its 5′-end. The DNA fragment has an effector segment that can partially block, partially inhibit, or partially reduce the rate of cleavage of reporter RNA by the Cas nuclease. FIG. 5B graphically illustrates signal strength from droplets having DNA-extended crRNAs, where the 5′ DNA extension has variable length and sequence. As shown, addition of two thymine nucleotides (2T), five thymine nucleotides (5T), or eight adenine nucleotides (8A) to 5′ end of crRNAs reduced the droplet signal significantly compared to the non-extended crRNA (−). The signal was undetectable when seven thymine nucleotides (7T) or twelve thymine nucleotides (12T) were linked the S′ end of the crRNA. Thus, Cas13's trans-cleavage rate was reduced to a greater extent when longer DNA extensions were used and when thymine (T) nucleotides were used rather than adenine (A) nucleotides in its effector region. FIG. 5C graphically illustrates that the number of positive droplets decreased only slightly when various DNA-crRNA 4 hybrids (e.g., two thymine nucleotide (2T), five thymine nucleotide (5T), or eight adenine nucleotide (8A) DNA extensions to the crRNA) were used instead of crRNA 4 (SEQ ID NO:4; indicated by a dash). FIG. 5D graphically illustrates signal intensities per droplet for droplets containing different viral target RNAs and different crRNAs. The crRNA 4 (SEQ ID NO:4) was used for SARS-CoV-2 wildtype (SC2 WT), a crRNA delta was used for SARS-CoV-2 delta (SC2 delta), a crRNA NL63 was used for HCoV-NL-63 (NL-63), and a crRNA H3N2 was used for H3N2 influenza virus (H3N2). FIG. 5E illustrates signal trajectories measured for droplets having SARS-CoV-2 wildtype (SC2 WT), SARS-CoV-2 delta (SC2 delta), HCoV-NL-63 (NL-63), and a H3N2 influenza virus (IAV H3N2) when different DNA fragments were linked to the crRNAs targeting these viral RNAs. The DNA fragments employed were an AT dinucleotide for SARS-CoV-2 delta (SC2 delta), a thymine dinucleotide for SARS-CoV-2 wildtype (SC2 WT), and a four thymine nucleotide oligo for H3N2 influenza virus (IAV H3N2). The 1-hour signals were measured from individual droplets containing individual target virus RNA and the graph to the right shows the proportion of droplets having particular normalized slope values. As illustrated, each viral target had a distinct normalized signal slope. FIG. 5F graphically illustrates the signal intensities over time for the viral targets and crRNAs used as described in FIG. 5D-5E. FIG. 5G graphically illustrates that the slope distributions differ for each viral target when the viral targets and all four crRNAs are combined into the same droplet. The same crRNAs and viral targets described in FIG. 5E were combined into droplets. As illustrated, each viral target had a distinct slope distribution, which was similar to those shown in FIG. SE. Note that there were two signal peaks for SARS-CoV-2 delta when the crRNAs were combined because both the crRNA 4 and the crRNA delta can target the SARS-CoV-2 delta RNA. FIG. 5H illustrates that combinations of crRNAs can be used to identify viruses in samples containing either one or two different viruses because the slope of the signals for the different viral RNA targets are distinct. The kinetic barcoding methods and assay mixtures not only correctly identified the virus target but also quantified the proportion of each infection among the possible single or dual infection scenarios.

FIG. 6 illustrates that the signal distribution amongst a population of droplets that include SARS-CoV-2 wild type (SC2 wt), SARS-CoV-2 delta (SC2 delta) variant, and SARS-CoV-2 omicron (SC2 omicron) target RNAs after incubation of the droplet population for 1 hour with three different crRNAs. The crRNA targeting SARS-CoV-2 wild type was linked at its 5′ end to a deoxynucleotide oligo with sequence AAAAAAAA. The crRNA targeting SARS-CoV-2 delta was linked at its 5′ end to two thymine deoxynucleotides. As illustrated, the variation in signal intensities and the proportion of droplets emitting a particular signal intensity was diagnostic of the type of SARS-CoV-2 RNA in the droplet population.

### DETAILED DESCRIPTION

Described herein are methods and assay compositions for detecting RNA targets using droplet assays. The droplets in the assays contain target-specific CRISPR guide RNAs (crRNAs) within Cas nuclease-crRNA ribonucleoprotein complexes that will cleave reporter RNA upon binding a target RNA, thereby generating fluorescence within the droplets that contain the target RNA. Not all of the droplets may contain the target RNA. The number of fluorescent droplets can be a measure of the concentration of target RNA in a sample.

Moreover, experiments described herein show that fluorescence generated by droplet-based Cas nuclease enzymatic activity is not always continuous and exhibits variable kinetics. The droplets are designed to encapsulate just a single target RNA. As demonstrated herein, the kinetics of fluorescence production by a particular droplet is a signature that uniquely identifies the target RNA. Because the droplets are designed to include a single RNA target, and the kinetics of fluorescence by many droplets can simultaneously be monitored, droplet-based Cas nuclease-crRNA assay procedures can be multiplexed to detect multiple target RNAs in a population of droplets. Such multiplexing can involve use of multiple crRNAs. When multiple crRNAs are used, they are used at equal concentrations so that a mixture of Cas nuclease-crRNA ribonucleoprotein complexes has approximately equal numbers of each type of crRNA-containing complexes.

As demonstrated herein, sometimes the Cas enzyme is actively cleaving the reporter RNA and producing fluorescence, and sometimes the Cas enzyme is not actively cleaving the reporter RNA, and therefore not producing fluorescence or producing less fluorescence than previously. These stochastic changes were observed, for example when the Cas protein/guide RNA was in the presence of targets with point mutations or different viral strains. The results show that the kinetics of the reaction are characteristic of the specific combination of Cas13, guide RNA, and target RNA. This means that by following the generation of a fluorescent signal from a single target molecule, the kinetics can be observed and the presence of a variant or mutant nucleic acid can be detected. This method is referred to herein as ‘kinetic barcoding.’

Assay mixtures are therefore described herein that can include a population of droplets. The mean diameter of the droplets can range from at least 10 to 60 μm. The droplet population including a test droplet subpopulation that includes at least one ribonucleoprotein (RNP) complex, plus at least one reporter RNA, plus at least one target RNA. In some cases, the population can include droplets that do not include one or more of a ribonucleoprotein complex, a reporter RNA, or a target RNA; these droplets can be used as control droplets. For example, the control droplets can be used to define background levels of fluorescence.

Also described herein are methods for detecting and/or identifying an RNA. Such methods can involve measuring and/or monitoring fluorescence of individual droplets in a population of droplets, where the population includes at least one droplet comprising a target RNA, a ribonucleoprotein complex, and at least one type of reporter RNA. Such a droplet population comprises at least two, at least three, at least five, at least seven, at least ten, at least fifteen, at least twenty, at least thirty, at least fifty droplets, each comprising a target RNA, a ribonucleoprotein complex, and at least one type of reporter RNA.

The target RNA can be the same target RNA throughout the population of droplets. However, in many cases different droplets each contain a different target RNA. The target RNAs can be one or more viral RNAs, prokaryotic RNAs, eukaryotic RNAs, or combinations thereof.

In some cases, at least one target RNA can be a wild type target RNA sequence. In some cases, at least one target RNA can be a variant or mutant target RNA sequence. Examples of target RNAs includes RNAs from one or more SARS coronaviruses (SARS-CoV-1 and/or SARS-CoV-2), Orthomyxoviruses (influenza viruses), Hepatitis C Viruses (HCVs), Ebola, influenza viruses, polio viruses, measles viruses, retroviruses, Human T-cell lymphotropic virus type 1 (HTLV-1), human immunodeficiency viruses (HIV), or combinations thereof. In some cases, at least one target RNA is a coronavirus RNA. In some cases, at least one target RNA can be an RNA for a disease marker. In some cases, at least one target RNA can be a microRNA.

The methods can also include (a) contacting a sample with at least one type of ribonucleoprotein (RNP) complex and at least one type of reporter RNA to form a reaction mixture; (b) mixing the reaction mixture with oil and surfactant to form an emulsion comprising droplets, where at least some of the droplets encapsulate an aqueous solution comprising the reaction mixture; (c) removing excess oil from the droplets; (d) selecting at least 1 droplet, or at least 3 droplets, or at least 10 droplets that emit fluorescence as positive droplets for monitoring; and (e) monitoring the fluorescence of the positive droplets over time.

The ribonucleoprotein (RNP) complex includes a Cas nuclease and a CRISPR guide RNA (crRNA). The Cas nuclease cleaves a reporter RNA when the RNP binds to its target via the crRNA. The kinetics of positive droplet fluorescence relates to the accessibility of the RNP for its target. Hence, selection of a crRNA affects the kinetics of fluorescence production within positive droplets. For example, the location of the crRNA binding site on the target RNA, or the presence of sequence mismatches can affect the kinetics of a positive droplet's fluorescence.

In some cases, the crRNA can be an RNA-polymer hybrid, wherein a polymer is covalently linked to the 5′-end of at least one crRNA. Such a polymer can inhibit or reduce the incidence of cleavage of at least one of the reporter RNAs. For example, the polymer can at least partially reduce the formation or activity of Cas nuclease higher-eukaryotes-and-prokaryotes nucleotide-binding (HEPN) domain. Use of such an RNA-polymer hybrid as a crRNA can slow down the production of signal from a droplet, which can improve identification of the different types of target RNAs in the assay mixture.

Polymers that can be used for RNA-polymer hybrid crRNAs can be polyethylene glycol, poly[oxy(11-(3-(9-adeninyl)propionato)-undecanyl-1-thiomethyl)ethylene] (PECH-AP), poly[oxy(11-(5-(9-adenylethyloxy)-4-oxopentanoato)undecanyl-1-thiomethyl)ethylene] (PECH-AS), single stranded DNA (e.g., having natural and/or unnatural linkages and/or natural and/or unnatural nucleotides), or a combination thereof.

In some cases the polymer includes a linker that is covalently linked to the crRNA 5′-end and a segment that at least partially reduce the Cas nuclease activity. For example, the linker can be a 6-10 nucleotide single-stranded DNA, or an eight nucleotide single-stranded DNA.

### Kinetics

The kinetics of fluorescence signals by droplets can be monitored by observing droplet fluorescence over time, for example by taking images of the droplet(s) at selected intervals. Droplets need not be monitored continuously but droplets do move, and individual droplets must be distinguished and identified from one imaging interval to the next. Hence, droplets can be identified by the track of their motion, for example, using a Kalman filter (e.g. in MATLAB) to predict the track's location in each image frame and to determine the likelihood that each detection within a series of image frames is being assigned to a particular tracked droplet. Only the droplets showing continuous trajectories in time and magnitude are selected for downstream analysis.

In some cases, images can be obtained after excitation of the fluorescent dye at intervals, for example, of 1 second to 5 minutes. In some cases, the images are obtained at intervals of 2 seconds to 4 minutes, or at intervals of 3 seconds to 3 minutes, or at intervals of 5 seconds to 1 minute. For example, in some of the experiments described herein, sixteen field-of-views (FOV) were acquired every 30 seconds for the time course of imaging and 36 field-of views were acquired for the endpoint imaging.

Several kinetic parameters can be used as ‘kinetic barcodes’ for identifying droplets and the targets encapsulated by those droplets. Individual signal trajectories can be evaluated by determining the slope of signal over time (slope), the time from target addition to the initiation of enzyme activity (Tᵢₙᵢₜ), and the root-mean-square-deviation (RMSD) from signal time trajectories by linear regression. Because some time was used to prepare the reaction mixtures and the droplet, a constant set-up time can be added to Tinit to reflect the time from droplet formation until the beginning of timed imaging. In addition, the time periods during which droplet's fluorescence signal increases quickly or slowly can be noted, and the percent ‘slopefast’ and ‘slopeslow’ parameters therefrom. For example, the slopefast and slopeslow parameters can be determined as a fraction or percent of time spent in each period, using a normal gaussian pdf (bell-curve) to obtain the instantaneous slope distribution. The slope, Tᵢₙᵢₜ, RMSD, slopefast, and slopeslow parameters are all kinetic parameters that individually or in combination can be used as a kinetic barcode that uniquely defines which crRNA/target combination is present within a particular droplet, or a particular subpopulation of droplets.

The kinetics can also be controlled in a programmable way by adding a polymer such as DNA to the crRNA such that the kinetics trans cleavage are modified. See further description below in the Ribonucleoproteins section.

### Samples

A variety of samples can be evaluated to ascertain whether one or more RNA molecules are present. The source of the samples can be any biological material. For example, the samples can be any biological fluid or tissue from any virus, fungus, plant or animal that is suspected of having an RNA. Examples of RNA types that can be evaluated in the methods include mRNAs, genomic RNAs, tRNAs, rRNAs, microRNAs, and combinations thereof. In some cases the RNA is a viral RNA, a mRNA marker for disease, a rRNA that could define what type of organism may be present in a sample, a microRNA that may silence gene function, or any other type of RNA.

In some cases, the samples can include a wild type target RNA sequence. In some cases, the samples can include at least one variant or mutant target RNA sequence. Samples can include RNAs (target RNAs) from one or more SARS coronaviruses (SARS-CoV-1 and/or SARS-CoV-2), Orthomyxoviruses (influenza viruses), Hepatitis C Viruses (HCVs), Ebola, influenza viruses, polio viruses, measles viruses, retroviruses, Human T-cell lymphotropic virus type 1 (HTLV-1), human immunodeficiency viruses (HIV), or combinations thereof. In some cases, the samples can include at least one coronavirus RNA. In some cases, the sample can include an RNA for a disease marker. In some cases, the sample can include a microRNA.

In some cases, it may not be known whether the biological sample contains RNA. However, such biological samples can still be tested using the methods described herein.

To obtain potential RNA from biological samples, the samples can be subjected to lysis, RNA extraction, inhibition of RNase(s), storage until testing is initiated, or other manipulations. In general, such manipulations are used to purify and preserve the RNA so that accurate kinetic barcoding can be performed.

### Ribonucleoproteins

As described herein, samples that are tested to determine the presence and/or type of a particular RNA are incubated with a ribonucleoprotein (RNP) complex that includes a Cas nuclease and a CRISPR guide RNA (crRNA). When a crRNA is present, the Cas nucleases employed bind and cleave RNA substrates, rather than DNA substrates, to which Cas9 can bind. The Cas nuclease can be one or more Cas12 or Cas13 (some previously known as C2c2) nuclease. For example, the Cas nuclease can be a Cas 13a nuclease, Cas13b nuclease, Cas13c nuclease, Cas13d nuclease, or a combination thereof.

The CRISPR guide RNAs (crRNAs) used in the assay mixtures and methods described herein can have approximately 64 nucleotides (e.g., 55-70 nucleotides). However, in some cases the crRNAs used in the assay mixtures and methods described herein can have more than 64 nucleotides because additional deoxynucleotides are added to the 5′ end of one or more of the crRNAs. For example, at least 6, or at least 7, or at least 8, or at least 9, or at least 10, or at least 11, or at least 12, or at least 13, or at least 14, or at least 15, or at least 16, or at least 17, or at least 18, or at least 19, or at least 20, or at least 21, or at least 22, or at least 23, or at least 24, or at least 25 , or at least 26, or at least 27, or at least 28 additional deoxynucleotides are added to the 5′ end of one or more of the crRNAs.

In some cases the crRNAs used in the assay mixtures and methods described herein can have approximately 64 nucleotides (e.g., 55-70 nucleotides) but have a polymer covalently bound to the 5′ end of one or more of the crRNAs.

Such added deoxynucleotides and/or polymers can inhibit or reduce the incidence of cleavage of at least one of the reporter RNAs. For example, the added deoxynucleotides and/or polymers can at least partially reduce the activity of the Cas nuclease, for example by sterically hindering the folding or activity of a higher-eukaryotes-and-prokaryotes nucleotide-binding (HEPN) domain. Use of such an RNA-polymer hybrid as a crRNA can slow down the production of signal from a droplet, which can improve identification of the different types of target RNAs in the assay mixture.

Polymers that can be used for RNA-polymer hybrid crRNAs can be polyethylene glycol, poly[oxy(11-(3-(9-adeninyl)propionato)-undecanyl-1-thiomethyl)ethylene] (PECH-AP), poly[oxy(11-(5-(9-adenylethyloxy)-4-oxopentanoato)undecanyl-1-thiomethyl)ethylene] (PECH-AS), single stranded DNA, or a combination thereof. In some cases the polymer includes a linker that is covalently linked to the crRNA 5′-end and a segment that at least partially reduces the Cas nuclease activity. For example, the linker can be a 6-10 nucleotide single-stranded DNA, or an eight nucleotide single-stranded DNA.

The crRNAs used in the assay mixtures and methods described herein include a “spacer” sequence of about 23 nucleotides, that is complementary to a portion of the target RNA.

The ribonucleoprotein (RNP) complex includes a Cas nuclease as well as a crRNA. In some cases, the Cas nucleases can be from a variety of organisms and can have sequence variations. For example, the Cas proteins can have at least 75%, at least 80%, at least 85%, at least 90%, at least 95%, at least 98%, at least 99%, or 100% sequence identity to any of the foregoing Cas 13 sequences from: Leptotrichia wadei, Leptotrichia buccalis, Rhodobacter capsulatus, Herbinix hemicellulosilytica, Leptotrichia buccalis (Lbu), Listeria seeligeri, Paludibacter propionicigenes, Lachnospiraceae bacterium, [Eubacterium] rectale, Listeria newyorkensis, Clostridium aminophilum, and/or Leptotrichia shahii.

For example, a Leptotrichia wadei Cas13a endonuclease can be used that has the following sequence (SEQ ID NO:71; NCBI accession no. WP_036059678.1).

| | |
|---|---|
| 1 | MKITKIDGVS HYKKQDKGIL KKKWKDLDER KQREKIEARY |
| 41 | NKQIESKIYK EFFRLKNKKR IEKEEDQNIK SLYFFIKELY |
| 81 | LNEKNEEWEL KNINLEILDD KERVIKGYKF KEDVYFFKEG |
| 121 | YKEYYLRILF NNLIEKVQNE NREKVRKNKE FLDLKEIFKK |
| 161 | YKNRKIDILL KSINNNKINL EYKKENVNEE IYGINPTNDR |
| 201 | EMTFYELLKE IIEKKDEQKS ILEEKLDNFD ITNFLENIEK |
| 241 | IFNEETEINI IKGKVLNELR EYIKEKEENN SDNKLKQIYN |
| 281 | LELKKYIENN FSYKKQKSKS KNGKNDYLYL NFLKKIMFIE |
| 321 | EVDEKKEINK EKFKNKINSN FKNLFVQHIL DYGKLLYYKE |
| 361 | NDEYIKNTGQ LETKDLEYIK TKETLIRKMA VLVSFAANSY |
| 401 | YNLFGRVSGD ILGTEVVKSS KTNVIKVGSH IFKEKMLNYF |
| 441 | FDFEIFDANK IVEILESISY SIYNVRNGVG HFNKLILGKY |
| 481 | KKKDINTNKR IEEDLNNNEE IKGYFIKKRG EIERKVKEKF |
| 521 | LSNNLQYYYS KEKIENYFEV YEFEILKRKI PFAPNFKRII |
| 561 | KKGEDLFNNK NNKKYEYFKN FDKNSAEEKK EFLKTRNFLL |
| 601 | KELYYNNFYK EFLSKKEEFE KIVLEVKEEK KSRGNINNKK |
| 641 | SGVSFQSIDD YDTKINISDY IASIHKKEME RVEKYNEEKQ |
| 681 | KDTAKYIRDF VEEIFLTGFI NYLEKDKRLH FLKEEFSILC |
| 721 | NNNNNVVDFN ININEEKIKE FLKENDSKTL NLYLFFNMID |
| 761 | SKRISEFRNE LVKYKQFTKK RLDEEKEFLG IKIELYETLI |
| 801 | EFVILTREKL DTKKSEEIDA WLVDKLYVKD SNEYKEYEEI |
| 841 | LKLFVDEKIL SSKEAPYYAT DNKTPILLSN FEKTRKYGTQ |
| 881 | SFLSEIQSNY KYSKVEKENI EDYNKKEEIE QKKKSNIEKL |
| 921 | QDLKVELHKK WEQNKITEKE IEKYNNTTRK INEYNYLKNK |
| 961 | EELQNVYLLH EMLSDLLARN VAFFNKWERD FKFIVIAIKQ |
| 1001 | FLRENDKEKV NEFLNPPDNS KGKKVYFSVS KYKNTVENID |
| 1041 | GIHKNFMNLI FLNNKFMNRK IDKMNCAIWV YFRNYIAHFL |
| 1081 | HLHTKNEKIS LISQMNLLIK LFSYDKKVQN HILKSTKTLL |
| 1121 | EKYNIQINFE ISNDKNEVTK YKIKNRLYSK KGKMLGKNNK |
| 1161 | LENEFLE NVKAMLEYSE |

Other sequences for Leptotrichia wadei Cas13a endonucleases are also available, such as those NCBI accession nos. BBM46759.1, BBM48616.1, BBM48974.1, BBM48975.1, and WP_021746003.1.

In another example, a Herbinix hemicellulosilytica Cas13a endonuclease can be used that has the following sequence (SEQ ID NO:72; NCBI accession no. WP_103203632.1).

| | |
|---|---|
| 1 | MKLTRRRISG NSVDQKITAA FYRDMSQGLL YYDSEDNDCT |
| 41 | DKVIESMDFE RSWRGRILKN GEDDKNPFYM FVKGLVGSND |
| 81 | KIVCEPIDVD SDPDNLDILI NKNLTGFGRN LKAPDSNDTL |
| 121 | ENLIRKIQAG IPEEEVLPEL KKIKEMIQKD IVNRKEQLLK |
| 161 | SIKNNRIPFS LEGSKLVPST KKMKWLFKLI DVPNKTFNEK |
| 201 | MLEKYWEIYD YDKLKANITN RLDKTDKKAR SISRAVSEEL |
| 241 | REYHKNLRTN YNRFVSGDRP AAGLDNGGSA KYNPDKEEFL |
| 281 | LFLKEVEQYF KKYFPVKSKH SNKSKDKSLV DKYKNYCSYK |
| 321 | VVKKEVNRSI INQLVAGLIQ QGKLLYYFYY NDTWQEDFLN |
| 361 | SYGLSYIQVE EAFKKSVMTS LSWGINRLTS FFIDDSNTVK |
| 401 | FDDITTKKAK EAIESNYFNK LRTCSRMQDH FKEKLAFFYP |
| 441 | VYVKDKKDRP DDDIENLIVL VKNAIESVSY LRNRTFHFKE |
| 481 | SSLLELLKEL DDKNSGQNKI DYSVAAEFIK RDIENLYDVF |
| 521 | REQIRSLGIA EYYKADMISD CFKTCGLEFA LYSPKNSLMP |
| 561 | AFKNVYKRGA NLNKAYIRDK GPKETGDQGQ NSYKALEEYR |
| 601 | ELTWYIEVKN NDQSYNAYKN LLQLIYYHAF LPEVRENEAL |
| 641 | ITDFINRTKE WNRKETEERL NTKNNKKHKN FDENDDITVN |
| 681 | TYRYESIPDY QGESLDDYLK VLQRKQMARA KEVNEKEEGN |
| 721 | NNYIQFIRDV VVWAFGAYLE NKLKNYKNEL QPPLSKENIG |
| 761 | LNDTLKELFP EEKVKSPFNI KCRFSISTFI DNKGKSTDNT |
| 801 | SAEAVKTDGK EDEKDKKNIK RKDLLCFYLF LRLLDENEIC |
| 841 | KLQHQFIKYR CSLKERRFPG NRTKLEKETE LLAELEELME |
| 881 | LVRFTMPSIP EISAKAESGY DTMIKKYFKD FIEKKVFKNP |
| 921 | KTSNLYYHSD SKTPVTRKYM ALLMRSAPLH LYKDIFKGYY |
| 961 | LITKKECLEY IKLSNIIKDY QNSLNELHEQ LERIKLKSEK |
| 1001 | QNGKDSLYLD KKDFYKVKEY VENLEQVARY KHLQHKINFE |
| 1041 | SLYRIFRIHV DIAARMVGYT QDWERDMHFL FKALVYNGVL |
| 1081 | EERRFEAIFN NNDDNNDGRI VKKIQNNLNN KNRELVSMLC |
| 1121 | WNKKLNKNEF GAIIWKRNPI AHLNHFTQTE QNSKSSLESL |
| 1161 | INSLRILLAY DRKRQNAVTK TINDLLLNDY HIRIKWEGRV |
| 1201 | DEGQIYFNIK EKEDIENEPI IHLKHLHKKD CYIYKNSYMF |
| 1241 | DKQKEWICNG IKEEVYDKSI LKCIGNLFKF DYEDKNKSSA |
| 1281 | NPKHT |

However, in some cases the Cas13 proteins with the SEQ ID NO:72 sequence are not used.

In another example, a Leptotrichia buccalis Cas13a endonuclease can be used that has the following sequence (SEQ ID NO:73; NCBI accession no. WP_015770004.1).

| | |
|---|---|
| 1 | MKVTKVGGIS HKKYTSEGRL VKSESEENRT DERLSALLNM |
| 41 | RLDMYIKNPS STETKENQKR IGKLKKFFSN KMVYLKDNTL |
| 81 | SLKNGKKENI DREYSETDIL ESDVRDKKNF AVLKKIYLNE |
| 121 | NVNSEELEVF RNDIKKKLNK INSLKYSFEK NKANYQKINE |
| 161 | NNIEKVEGKS KRNIIYDYYR ESAKRDAYVS NVKEAFDKLY |
| 201 | KEEDIAKLVL EIENLTKLEK YKIREFYHEI IGRKNDKENF |
| 241 | AKIIYEEIQN VNNMKELIEK VPDMSELKKS QVFYKYYLDK |
| 281 | EELNDKNIKY AFCHFVEIEM SQLLKNYVYK RLSNISNDKI |
| 321 | KRIFEYQNLK KLIENKLLNK LDTYVRNCGK YNYYLQDGEI |
| 361 | ATSDFIARNR QNEAFLRNII GVSSVAYFSL RNILETENEN |
| 401 | DITGRMRGKT VKNNKGEEKY VSGEVDKIYN ENKKNEVKEN |
| 441 | LKMFYSYDFN MDNKNEIEDF FANIDEAISS IRHGIVHFNL |
| 481 | ELEGKDIFAF KNIAPSEISK KMFQNEINEK KLKLKIFRQL |
| 521 | NSANVFRYLE KYKILNYLKR TRFEFVNKNI PFVPSFTKLY |
| 561 | SRIDDLKNSL GIYWKTPKTN DDNKTKEIID AQIYLLKNIY |
| 601 | YGEFLNYFMS NNGNFFEISK EIIELNKNDK RNLKTGFYKL |
| 641 | QKFEDIQEKI PKEYLANIQS LYMINAGNQD EEEKDTYIDF |
| 681 | IQKIFLKGFM TYLANNGRLS LIYIGSDEET NTSLAEKKQE |
| 721 | FDKFLKKYEQ NNNIKIPYEI NEFLREIKLG NILKYTERLN |
| 761 | MFYLILKLLN HKELTNLKGS LEKYQSANKE EAFSDQLELI |
| 801 | NLLNLDNNRV TEDFELEADE IGKFLDFNGN KVKDNKELKK |
| 841 | FDTNKIYFDG ENIIKHRAFY NIKKYGMLNL LEKIADKAGY |
| 881 | KISIEELKKY SNKKNEIEKN HKMQENLHRK YARPRKDEKF |
| 921 | TDEDYESYKQ AIENIEEYTH LKNKVEFNEL NLLQGLLLRI |
| 961 | LHRLVGYTSI WERDLRFRLK GEFPENQYIE EIFNFENKKN |
| 1001 | VKYKGGQIVE KYIKFYKELH QNDEVKINKY SSANIKVLKQ |
| 1041 | EKKDLYIRNY IAHFNYIPHA EISLLEVLEN LRKLLSYDRK |
| 1081 | LKNAVMKSVV DILKEYGFVA TFKIGADKKI GIQTLESEKI |
| 1121 | VHLKNLKKKK LMTDRNSEEL CKLVKIMFEY KMEEKKSEN |

However, in some cases the Cas13 proteins with the SEQ ID NO:73 sequence are not used.

In another example, a Leptotrichia seeligeri Cas13a endonuclease can be used that has the following sequence (SEQ ID NO:74; NCBI accession no. WP_012985477.1).

| | |
|---|---|
| 1 | MWISIKTLIH HLGVLFFCDY MYNRREKKII EVKTMRITKV |
| 41 | EVDRKKVLIS RDKNGGKLVY ENEMQDNTEQ IMHHKKSSFY |
| 81 | KSVVNKTICR PEQKQMKKLV HGLLQENSQE KIKVSDVTKL |
| 121 | NISNFLNHRF KKSLYYFPEN SPDKSEEYRI EINLSQLLED |
| 161 | SLKKQQGTFI CWESFSKDME LYINWAENYI SSKTKLIKKS |
| 201 | IRNNRIQSTE SRSGQLMDRY MKDILNKNKP FDIQSVSEKY |
| 241 | QLEKLTSALK ATFKEAKKND KEINYKLKST LQNHERQIIE |
| 281 | ELKENSELNQ FNIEIRKHLE TYFPIKKTNR KVGDIRNLEI |
| 321 | GEIQKIVNHR LKNKIVQRIL QEGKLASYEI ESTVNSNSLQ |
| 361 | KIKIEEAFAL KFINACLFAS NNLRNMVYPV CKKDILMIGE |
| 401 | FKNSFKEIKH KKFIRQWSQF FSQEITVDDI ELASWGLRGA |
| 441 | IAPIRNEIIH LKKHSWKKFF NNPTFKVKKS KIINGKTKDV |
| 481 | TSEFLYKETL FKDYFYSELD SVPELIINKM ESSKILDYYS |
| 521 | SDQLNQVFTI PNFELSLLTS AVPFAPSFKR VYLKGFDYQN |
| 561 | QDEAQPDYNL KLNIYNEKAF NSEAFQAQYS LFKMVYYQVF |
| 601 | LPQFTTNNDL FKSSVDFILT LNKERKGYAK AFQDIRKMNK |
| 641 | DEKPSEYMSY IQSQLMLYQK KQEEKEKINH FEKFINQVFI |
| 681 | KGFNSFIEKN RLTYICHPTK NTVPENDNIE IPFHTDMDDS |
| 721 | NIAFWLMCKL LDAKQLSELR NEMIKFSCSL QSTEEISTFT |
| 761 | KAREVIGLAL LNGEKGCNDW KELFDDKEAW KKNMSLYVSE |
| 801 | ELLQSLPYTQ EDGQTPVINR SIDLVKKYGT ETILEKLFSS |
| 841 | SDDYKVSAKD IAKLHEYDVT EKIAQQESLH KQWIEKPGLA |
| 881 | RDSAWTKKYQ NVINDISNYQ WAKTKVELTQ VRHLHQLTID |
| 921 | LLSRLAGYMS IADRDFQFSS NYILERENSE YRVTSWILLS |
| 961 | ENKNKNKYND YELYNLKNAS IKVSSKNDPQ LKVDLKQLRL |
| 1001 | TLEYLELFDN RLKEKRNNIS HFNYLNGQLG NSILELFDDA |
| 1041 | RDVLSYDRKL KNAVSKSLKE ILSSHGMEVT FKPLYQTNHH |
| 1081 | LKIDKLQPKK IHHLGEKSTV SSNQVSNEYC QLVRTLLTMK |

For example, a Paludibacter propionicigenes Cas13a endonuclease can be used that has the following sequence (SEQ ID NO. 75; NCBI accession no. WP_013443710.1)

| | |
|---|---|
| 1 | MRVSKVKVKD GGKDKMVLVH RKTTGAQLVY SGQPVSNETS |
| 41 | NILPEKKRQS FDLSTLNKTI IKFDTAKKQK LNVDQYKIVE |
| 81 | KIFKYPKQEL PKQIKAEEIL PFLNHKFQEP VKYWKNGKEE |
| 121 | SFNLTLLIVE AVQAQDKRKL QPYYDWKTWY IQTKSDLLKK |
| 161 | SIENNRIDLT ENLSKRKKAL LAWETEFTAS GSIDLTHYHK |
| 201 | VYMTDVLCKM LQDVKPLTDD KGKINTNAYH RGLKKALQNH |
| 241 | QPAIFGTREV PNEANRADNQ LSIYHLEVVK YLEHYFPIKT |
| 281 | SKRRNTADDI AHYLKAQTLK TTIEKQLVNA IRANIIQQGK |
| 321 | TNHHELKADT TSNDLIRIKT NEAFVLNLTG TCAFAANNIR |
| 361 | NMVDNEQTND ILGKGDFIKS LLKDNTNSQL YSFFFGEGLS |
| 401 | TNKAEKETQL WGIRGAVQQI RNNVNHYKKD ALKTVFNISN |
| 441 | FENPTITDPK QQTNYADTIY KARFINELEK IPEAFAQQLK |
| 481 | TGGAVSYYTI ENLKSLLTTF QFSLCRSTIP FAPGFKKVFN |
| 521 | GGINYQNAKQ DESFYELMLE QYLRKENFAE ESYNARYFML |
| 561 | KLIYNNLFLP GFTTDRKAFA DSVGFVQMQN KKQAEKVNPR |
| 601 | KKEAYAFEAV RPMTAADSIA DYMAYVQSEL MQEQNKKEEK |
| 641 | VAEETRINFE KFVLQVFIKG FDSFLRAKEF DFVQMPQPQL |
| 681 | TATASNQQKA DKLNQLEASI TADCKLTPQY AKADDATHIA |
| 721 | FYVFCKLLDA AHLSNLRNEL IKFRESVNEF KFHHLLEITE |
| 761 | ICLLSADVVP TDYRDLYSSE ADCLARLRPF IEQGADITNW |
| 801 | SDLFVQSDKH SPVIHANIEL SVKYGTTKLL EQIINKDTQF |
| 841 | KTTEANFTAW NTAQKSIEQL IKQREDHHEQ WVKAKNADDK |
| 881 | EKQERKREKS NFAQKFIEKH GDDYLDICDY INTYNWLDNK |
| 921 | MHFVHLNRLH GLTIELLGRM AGFVALFDRD FQFFDEQQIA |
| 961 | DEFKLHGFVN LHSIDKKINE VPTKKIKEIY DIRNKIIQIN |
| 1001 | GNKINESVRA NLIQFISSKR NYYNNAFLHV SNDEIKEKQM |
| 1041 | YDIRNHIAHF NYLTKDAADF SLIDLINELR ELLHYDRKLK |
| 1081 | NAVSKAFIDL FDKHGMILKL KLNADHKLKV ESLEPKKIYH |
| 1121 | LGSSAKDKPE YQYCTNQVMM AYCNMCRSLL EMKK |

For example, a Lachnospiraceae bacterium Cas13a endonuclease can be used that has the following sequence (SEQ ID NO:76, NCBI accession no. WP_022785443.1).

| | |
|---|---|
| 1 | MKISKVREEN RGAKLTVNAK TAVVSENRSQ EGILYNDPSR |
| 41 | YGKSRKNDED RDRYIESRLK SSGKLYRIFN EDKNKRETDE |
| 81 | LQWFLSEIVK KINRRNGLVL SDMLSVDDRA FEKAFEKYAE |
| 121 | LSYTNRRNKV SGSPAFETCG VDAATAERLK GIISETNFIN |
| 161 | RIKNNIDNKV SEDIIDRIIA KYLKKSLCRE RVKRGLKKLL |
| 201 | MNAFDLPYSD PDIDVQRDFI DYVLEDFYHV RAKSQVSRSI |
| 241 | KNMNMPVQPE GDGKFAITVS KGGTESGNKR SAEKEAFKKF |
| 281 | LSDYASLDER VRDDMLRRMR RLVVLYFYGS DDSKLSDVNE |
| 321 | KFDVWEDHAA RRVDNREFIK LPLENKLANG KTDKDAERIR |
| 361 | KNTVKELYRN QNIGCYRQAV KAVEEDNNGR YFDDKMLNMF |
| 401 | FIHRIEYGVE KIYANLKQVT EFKARTGYLS EKIWKDLINY |
| 441 | ISIKYIAMGK AVYNYAMDEL NASDKKEIEL GKISEEYLSG |
| 481 | ISSFDYELIK AEEMLQRETA VYVAFAARHL SSQTVELDSE |
| 521 | NSDFLLLKPK GTMDKNDKNK LASNNILNFL KDKETLRDTI |
| 561 | LQYFGGHSLW TDFPFDKYLA GGKDDVDFLT DLKDVIYSMR |
| 601 | NDSFHYATEN HNNGKWNKEL ISAMFEHETE RMTVVMKDKF |
| 641 | YSNNLPMFYK NDDLKKLLID LYKDNVERAS QVPSFNKVFV |
| 681 | RKNFPALVRD KDNLGIELDL KADADKGENE LKFYNALYYM |
| 721 | FKEIYYNAFL NDKNVRERFI TKATKVADNY DRNKERNLKD |
| 761 | RIKSAGSDEK KKLREQLQNY IAENDFGQRI KNIVQVNPDY |
| 801 | TLAQICQLIM TEYNQQNNGC MQKKSAARKD INKDSYQHYK |
| 841 | MLLLVNLRKA FLEFIKENYA FVLKPYKHDL CDKADFVPDF |
| 881 | AKYVKPYAGL ISRVAGSSEL QKWYIVSRFL SPAQANHMLG |
| 921 | FLHSYKQYVW DIYRRASETG TEINHSIAED KIAGVDITDV |
| 961 | DAVIDLSVKL CGTISSEISD YFKDDEVYAE YISSYLDFEY |
| 1001 | DGGNYKDSLN RFCNSDAVND QKVALYYDGE HPKLNRNIIL |
| 1041 | SKLYGERRFL EKITDRVSRS DIVEYYKLKK ETSQYQTKGI |
| 1081 | FDSEDEQKNI KKFQEMKNIV EFRDLMDYSE IADELQGQLI |
| 1121 | NWIYLRERDL MNFQLGYHYA CLNNDSNKQA TYVTLDYQGK |
| 1161 | KNRKINGAIL YQICAMYING LPLYYVDKDS SEWTVSDGKE |
| 1201 | STGAKIGEFY RYAKSFENTS DCYASGLEIF ENISEHDNIT |
| 1241 | ELRNYIEHFR YYSSFDRSFL GIYSEVFDRF FTYDLKYRKN |
| 1281 | VPTILYNILL QHFVNVRFEF VSGKKMIGID KKDRKIAKEK |
| 1321 | ECARITIREK NGVYSEQFTY KLKNGTVYVD ARDKRYLQSI |
| 1361 | IRLLFYPEKV NMDEMIEVKE KKKPSDNNTG KGYSKRDRQQ |
| 1401 | DRKEYDKYKE KKKKEGNFLS GMGGNINWDE INAQLKN |

For example, a Leptotrichia shahii Cas13a endonuclease can be used that has the following sequence (SEQ ID NO:77, NCBI accession no. BBM39911.1).

| | |
|---|---|
| 1 | MGNLFGHKRW YEVRDKKDFK IKRKVKVKRN YDGNKYILNI |
| 41 | NENNNKEKID NNKFIRKYIN YKKNDNILKE FTRKFHAGNI |
| 81 | LFKLKGKEGI IRIENNDDFL ETEEVVLYIE AYGKSEKLKA |
| 121 | LGITKKKIID EAIRQGITKD DKKIEIKRQE NEEEIEIDIR |
| 161 | DEYTNKTLND CSIILRIIEN DELETKKSIY EIFKNINMSL |
| 201 | YKITEKIIEN ETEKVFENRY YEEHLREKLL KDDKIDVILT |
| 241 | NFMEIREKIK SNLEILGFVK FYLNVGGDKK KSKNKKMLVE |
| 281 | KILNINVDLT VEDIADFVIK ELEFWNITKR IEKVKKVNNE |
| 321 | FLEKRRNRTY IKSYVLLDKH EKFKIERENK KDKIVKFFVE |
| 361 | NIKNNSIKEK IEKILAEFKI DELIKKLEKE LKKGNCDTEI |
| 401 | FGIFKKHYKV NFDSKKFSKK SDEEKELYKI IYRYLKGRIE |
| 441 | KILVNEQKVR LKKMEKIEIE KILNESILSE KILKRVKQYT |
| 481 | LEHIMYLGKL RHNDIDMTTV NTDDFSRLHA KEELDLELIT |
| 521 | FFASTNMELN KIFSRENINN DENIDFFGGD REKNYVLDKK |
| 561 | ILNSKIKIIR DLDFIDNKNN ITNNFIRKFT KIGTNERNRI |
| 601 | LHAISKERDL QGTQDDYNKV INIIQNLKIS DEEVSKALNL |
| 641 | DVVFKDKKNI ITKINDIKIS EENNNDIKYL PSFSKVLPEI |
| 681 | LNLYRNNPKN EPFDTIETEK IVLNALIYVN KELYKKLILE |
| 721 | DDLEENESKN IFLQELKKTL GNIDEIDENI IENYYKNAQI |
| 761 | SASKGNNKAI KKYQKKVIEC YIGYLRKNYE ELFDFSDFKM |
| 801 | NIQEIKKQIK DINDNKTYER ITVKTSDKTI VINDDFEYII |
| 841 | SIFALLNSNA VINKIRNRFF ATSVWLNTSE YQNIIDILDE |
| 881 | IMQLNTLRNE CITENWNLNL EEFIQKMKEI EKDFDDFKIQ |
| 921 | TKKEIFNNYY EDIKNNILTE FKDDINGCDV LEKKLEKIVI |
| 961 | FDDETKFEID KKSNILQDEQ RKLSNINKKD LKKKVDQYIK |
| 1001 | DKDQEIKSKI LCRIIFNSDF LKKYKKEIDN LIEDMESENE |
| 1041 | NKFQEIYYPK ERKNELYIYK KNLFLNIGNP NFDKIYGLIS |
| 1081 | NDIKMADAKF LFNIDGKNIR KNKISEIDAI LKNLNDKLNG |
| 1121 | YSKEYKEKYI KKLKENDDFF AKNIQNKNYK SFEKDYNRVS |
| 1161 | EYKKIRDLVE FNYLNKIESY LIDINWKLAI QMARFERDMH |
| 1201 | YIVNGLRELG IIKLSGYNTG ISRAYPKRNG SDGFYTTTAY |
| 1241 | YKFFDEESYK KFEKICYGFG IDLSENSEIN KPENESIRNY |
| 1281 | ISHFYIVRNP FADYSIAEQI DRVSNLLSYS TRYNNSTYAS |
| 1321 | VFEVFKKDVN LDYDELKKKF KLIGNNDILE RLMKPKKVSV |
| 1361 | LELESYNSDY IKNLIIELLT KIENTNDTL |

In another example, a Leptotrichia buccalis C-1013-b Cas13a endonuclease can have the following sequence (SEQ ID NO:78; NCBI accession no. C7NBY4; AltName LbuC2c2).

| | |
|---|---|
| 1 | MKVTKVGGIS HKKYTSEGRL VKSESEENRT DERLSALLNM |
| 41 | RLDMYIKNPS STETKENQKR IGKLKKFFSN KMVYLKDNTL |
| 81 | SLKNGKKENI DREYSETDIL ESDVRDKKNF AVLKKIYLNE |
| 121 | NVNSEELEVF RNDIKKKLNK INSLKYSFEK NKANYQKINE |
| 161 | NNIEKVEGKS KRNIIYDYYR ESAKRDAYVS NVKEAFDKLY |
| 201 | KEEDIAKLVL EIENLTKLEK YKIREFYHEI IGRKNDKENF |
| 241 | AKIIYEEIQN VNNMKELIEK VPDMSELKKS QVFYKYYLDK |
| 281 | EELNDKNIKY AFCHFVEIEM SQLLKNYVYK RLSNISNDKI |
| 321 | KRIFEYQNLK KLIENKLLNK LDTYVRNCGK YNYYLQDGEI |
| 361 | ATSDFIARNR QNEAFLRNII GVSSVAYFSL RNILETENEN |
| 401 | DITGRMRGKT VKNNKGEEKY VSGEVDKIYN ENKKNEVKEN |
| 441 | LKMFYSYDFN MDNKNEIEDF FANIDEAISS IRHGIVHFNL |
| 481 | ELEGKDIFAF KNIAPSEISK KMFQNEINEK KLKLKIFRQL |
| 521 | NSANVFRYLE KYKILNYLKR TRFEFVNKNI PFVPSFTKLY |
| 561 | SRIDDLKNSL GIYWKTPKTN DDNKTKEIID AQIYLLKNIY |
| 601 | YGEFLNYFMS NNGNFFEISK EIIELNKNDK RNLKTGFYKL |
| 641 | QKFEDIQEKI PKEYLANIQS LYMINAGNQD EEEKDTYIDF |
| 681 | IQKIFLKGFM TYLANNGRLS LIYIGSDEET NTSLAEKKQE |
| 721 | FDKFLKKYEQ NNNIKIPYEI NEFLREIKLG NILKYTERLN |
| 761 | MFYLILKLLN HKELTNLKGS LEKYQSANKE EAFSDQLELI |
| 801 | NLLNLDNNRV TEDFELEADE IGKFLDFNGN KVKDNKELKK |
| 841 | FDTNKIYFDG ENIIKHRAFY NIKKYGMLNL LEKIADKAGY |
| 881 | KISIEELKKY SNKKNEIEKN HKMQENLHRK YARPRKDEKF |
| 921 | TDEDYESYKQ AIENIEEYTH LKNKVEFNEL NLLQGLLLRI |
| 961 | LHRLVGYTSI WERDLRFRLK GEFPENQYIE EIFNFENKKN |
| 1001 | VKYKGGQIVE KYIKFYKELH QNDEVKINKY SSANIKVLKQ |
| 1041 | EKKDLYIRNY IAHFNYIPHA EISLLEVLEN LRKLLSYDRK |
| 1081 | LKNAVMKSVV DILKEYGFVA TFKIGADKKI GIQTLESEKI |
| 1121 | VHLKNLKKKK LMTDRNSEEL CKLVKIMFEY KMEEKKSEN |

In some cases, a modified Cas13 protein can be used. Such a modified Cas 13 protein can have increased in vivo endonuclease activity compared to a corresponding unmodified Cas13 protein. The modified Cas13 proteins, which can increase sensitivity of detecting at least one reporter RNA by about 10-fold to 100-fold are useful, for example, in the methods, kits, systems and devices described herein.

The inventors have evaluated the kinetics of other Cas13a and Cas13b proteins. Such work indicates that in some cases Cas13b works faster in the SARS-CoV-2 RNA detection assay than Cas13a.

For example, a Cas13b from Prevotella buccae can be used in the SARS-CoV-2 RNA detection methods, compositions and devices. A sequence for a Prevotella buccaeCas13b protein (NCBI accession no. WP_004343973.1) is shown below as SEQ ID NO:79.

| | |
|---|---|
| 1 | MQKQDKLFVD RKKNAIFAFP KYITIMENKE KPEPIYYELT |
| 41 | DKHFWAAFLN LARHNVYTTI NHINRRLEIA ELKDDGYMMG |
| 81 | IKGSWNEQAK KLDKKVRLRD LIMKHFPFLE AAAYEMTNSK |
| 121 | SPNNKEQREK EQSEALSLNN LKNVLFIFLE KLQVLRNYYS |
| 161 | HYKYSEESPK PIFETSLLKN MYKVFDANVR LVKRDYMHHE |
| 201 | NIDMQRDFTH LNRKKQVGRT KNIIDSPNFH YHFADKEGNM |
| 241 | TIAGLLFFVS LFLDKKDAIW MQKKLKGFKD GRNLREQMTN |
| 281 | EVFCRSRISI PKLKLENVQT KDWMQLDMLN ELVRCPKSLY |
| 321 | ERLREKDRES FKVPFDIFSD DYNAEEEPFK NTLVRHQDRF |
| 361 | PYFVLRYFDL NEIFEQLRFQ IDLGTYHFSI YNKRIGDEDE |
| 401 | VRHLTHHLYG FARIQDFAPQ NQPEEWRKLV KDLDHFETSQ |
| 441 | EPYISKTAPH YHLENEKIGI KFCSAHNNLF PSLQTDKTCN |
| 481 | GRSKFNLGTQ FTAEAFLSVH ELLPMMFYYL LLTKDYSRKE |
| 521 | SADKVEGIIR KEISNIYAIY DAFANNEINS IADLTRRLQN |
| 561 | TNILQGHLPK QMISILKGRQ KDMGKEAERK IGEMIDDTQR |
| 601 | RLDLLCKQTN QKIRIGKRNA GLLKSGKIAD WLVNDMMRFQ |
| 641 | PVQKDQNNIP INNSKANSTE YRMLQRALAL FGSENFRLKA |
| 681 | YFNQMNLVGN DNPHPFLAET QWEHQTNILS FYRNYLEARK |
| 721 | KYLKGLKPQN WKQYQHFLIL KVQKTNRNTL VTGWKNSFNL |
| 761 | PRGIFTQPIR EWFEKHNNSK RIYDQILSFD RVGFVAKAIP |
| 801 | LYFAEEYKDN VQPFYDYPFN IGNRLKPKKR QFLDKKERVE |
| 841 | LWQKNKELFK NYPSEKKKTD LAYLDFLSWK KFERELRLIK |
| 881 | NQDIVTWLMF KELFNMATVE GLKIGEIHLR DIDTNTANEE |
| 921 | SNNILNRIMP MKLPVKTYET DNKGNILKER PLATFYIEET |
| 961 | ETKVLKQGNF KALVKDRRLN GLFSFAETTD LNLEEHPISK |
| 1001 | LSVDLELIKY QTTRISIFEM TLGLEKKLID KYSTLPTDSF |
| 1041 | RNMLERWLQC KANRPELKNY VNSLIAVRNA FSHNQYPMYD |
| 1081 | ATLFAEVKKF TLFPSVDTKK IELNIAPQLL EIVGKAIKEI |
| 1121 | EKSENKN |

Such a Prevotella buccae Cas13b protein can have a Km (Michaelis constant) substrate concentration of about 20 micromoles and a Kcat of about 987/second (see, e.g., Slaymaker et al. Cell Rep 26 (13): 3741-3751 (2019)).

Another Prevotella buccae Cas13b protein (NCBI accession no. WP_004343581.1) that can be used in the SARS-CoV-2 RNA detection methods, compositions and devices has the sequence shown below as SEQ ID NO:80.

| | |
|---|---|
| 1 | MQKQDKLFVD RKKNAIFAFP KYITIMENQE KPEPIYYELT |
| 41 | DKHFWAAFLN LARHNVYTTI NHINRRLEIA ELKDDGYMMD |
| 81 | IKGSWNEQAK KLDKKVRLRD LIMKHFPFLE AAAYEITNSK |
| 121 | SPNNKEQREK EQSEALSLNN LKNVLFIFLE KLQVLRNYYS |
| 161 | HYKYSEESPK PIFETSLLKN MYKVFDANVR LVKRDYMHHE |
| 201 | NIDMQRDFTH LNRKKQVGRT KNIIDSPNFH YHFADKEGNM |
| 241 | TIAGLLFFVS LFLDKKDAIW MQKKLKGFKD GRNLREQMTN |
| 281 | EVFCRSRISL PKLKLENVQT KDWMQLDMLN ELVRCPKSLY |
| 321 | ERLREKDRES FKVPFDIFSD DYDAEEEPFK NTLVRHQDRF |
| 361 | PYFVLRYFDL NEIFEQLRFQ IDLGTYHFSI YNKRIGDEDE |
| 401 | VRHLTHHLYG FARIQDFAQQ NQPEVWRKLV KDLDYFEASQ |
| 441 | EPYIPKTAPH YHLENEKIGI KFCSTHNNLF PSLKTEKTCN |
| 481 | GRSKFNLGTQ FTAEAFLSVH ELLPMMFYYL LLTKDYSRKE |
| 521 | SADKVEGIIR KEISNIYAIY DAFANGEINS IADLTCRLQK |
| 561 | TNILQGHLPK QMISILEGRQ KDMEKEAERK IGEMIDDTQR |
| 601 | RLDLLCKQTN QKIRIGKRNA GLLKSGKIAD WLVNDMMRFQ |
| 641 | PVQKDQNNIP INNSKANSTE YRMLQRALAL FGSENFRLKA |
| 681 | YFNQMNLVGN DNPHPFLAET QWEHQTNILS FYRNYLEARK |
| 721 | KYLKGLKPQN WKQYQHFLIL KVQKTNRNTL VTGWKNSFNL |
| 761 | PRGIFTQPIR EWFEKANNSK RIYDQILSED RVGFVAKAIP |
| 801 | LYFAEEYKDN VQPFYDYPFN IGNKLKPQKG QFLDKKERVE |
| 841 | LWQKNKELFK NYPSEKKKTD LAYLDFLSWK KFERELRLIK |
| 881 | NQDIVTWLMF KELFNMATVE GLKIGEIHLR DIDTNTANEE |
| 921 | SNNILNRIMP MKLPVKTYET DNKGNILKER PLATFYIEET |
| 961 | ETKVLKQGNF KVLAKDRRLN GLLSFAETTD IDLEKNPITK |
| 1001 | LSVDHELIKY QTTRISIFEM TLGLEKKLIN KYPTLPTDSF |
| 1041 | RNMLERWLQC KANRPELKNY VNSLIAVRNA FSHNQYPMYD |
| 1081 | ATLFAEVKKF TLFPSVDTKK IELNIAPQLL EIVGKAIKEI |
| 1121 | EKSENKN |

An example of a Bergeyella zoohelcum Cas13b (R1177A) mutant sequence (NCBI accession no. 6AAY_A) is shown below as SEQ ID NO:81.

| | |
|---|---|
| 1 | XENKTSLGNN IYYNPFKPQD KSYFAGYFNA AXENTDSVFR |
| 41 | ELGKRLKGKE YTSENFFDAI FKENISLVEY ERYVKLLSDY |
| 81 | FPXARLLDKK EVPIKERKEN FKKNFKGIIK AVRDLRNFYT |
| 121 | HKEHGEVEIT DEIFGVLDEX LKSTVLTVKK KKVKTDKTKE |
| 161 | ILKKSIEKQL DILCQKKLEY LRDTARKIEE KRRNQRERGE |
| 201 | KELVAPFKYS DKRDDLIAAI YNDAFDVYID KKKDSLKESS |
| 241 | KAKYNTKSDP QQEEGDLKIP ISKNGVVFLL SLFLTKQEIH |
| 281 | AFKSKIAGFK ATVIDEATVS EATVSHGKNS ICFXATHEIF |
| 321 | SHLAYKKLKR KVRTAEINYG EAENAEQLSV YAKETLXXQX |
| 361 | LDELSKVPDV VYQNLSEDVQ KTFIEDWNEY LKENNGDVGT |
| 401 | XEEEQVIHPV IRKRYEDKFN YFAIRFLDEF AQFPTLRFQV |
| 441 | HLGNYLHDSR PKENLISDRR IKEKITVFGR LSELEHKKAL |
| 481 | FIKNTETNED REHYWEIFPN PNYDFPKENI SVNDKDFPIA |
| 521 | GSILDREKQP VAGKIGIKVK LLNQQYVSEV DKAVKAHQLK |
| 561 | QRKASKPSIQ NIIEEIVPIN ESNPKEAIVF GGQPTAYLSX |
| 601 | NDIHSILYEF FDKWEKKKEK LEKKGEKELR KEIGKELEKK |
| 641 | IVGKIQAQIQ QIIDKDTNAK ILKPYQDGNS TAIDKEKLIK |
| 681 | DLKQEQNILQ KLKDEQTVRE KEYNDFIAYQ DKNREINKVR |
| 721 | DRNHKQYLKD NLKRKYPEAP ARKEVLYYRE KGKVAVWLAN |
| 761 | DIKRFXPTDF KNEWKGEQHS LLQKSLAYYE QCKEELKNLL |
| 801 | PEKVFQHLPF KLGGYFQQKY LYQFYTCYLD KRLEYISGLV |
| 841 | QQAENFKSEN KVFKKVENEC FKFLKKQNYT HKELDARVQS |
| 881 | ILGYPIFLER GFXDEKPTII KGKTFKGNEA LFADWFRYYK |
| 921 | EYQNFQTFYD TENYPLVELE KKQADRKRKT KIYQQKKNDV |
| 961 | FTLLXAKHIF KSVFKQDSID QFSLEDLYQS REERLGNQER |
| 1001 | ARQTGERNTN YIWNKTVDLK LCDGKITVEN VKLKNVGDFI |
| 1041 | KYEYDQRVQA FLKYEENIEW QAFLIKESKE EENYPYVVER |
| 1081 | EIEQYEKVRR EELLKEVHLI EEYILEKVKD KEILKKGDNQ |
| 1121 | NFKYYILNGL LKQLKNEDVE SYKVFNLNTE PEDVNINQLK |
| 1161 | QEATDLEQKA FVLTYIANKF AHNQLPKKEF WDYCQEKYGK |
| 1201 | IEKEKTYAEY FAEVEKKEKE ALIKLEHHHH HH |

Another example of a Cas 1 3b protein sequence from Prevotella sp. MSX73 (NCBI accession no. WP_007412163.1) that can be used in the SARS-CoV-2 RNA detection methods, compositions and devices has is shown below as SEQ ID NO:82

| | |
|---|---|
| 1 | MQKQDKLFVD RKKNAIFAFP KYITIMENQE KPEPIYYELT |
| 41 | DKHFWAAFLN LARHNVYTTI NHINRRLEIA ELKDDGYMMG |
| 81 | IKGSWNEQAK KLDKKVRLRD LIMKHFPFLE AAAYEITNSK |
| 121 | SPNNKEQREK EQSEALSLNN LKNVLFIFLE KLQVLRNYYS |
| 161 | HYKYSEESPK PIFETSLLKN MYKVFDANVR LVKRDYMHHE |
| 201 | NIDMQRDFTH LNRKKQVGRT KNIIDSPNFH YHFADKEGNM |
| 241 | TIAGLLFFVS LFLDKKDAIW MQKKLKGFKD GRNLREQMTN |
| 281 | EVFCRSRISL PKLKLENVQT KDWMQLDMLN ELVRCPKSLY |
| 321 | ERLREKDRES FKVPEDIFSD DYDAEEEPFK NTLVRHQDRF |
| 361 | PYFVLRYFDL NEIFEQLRFQ IDLGTYHFSI YNKRIGDEDE |
| 401 | VRHLTHHLYG FARIQDFAPQ NQPEEWRKLV KDLDHFETSQ |
| 441 | EPYISKTAPH YHLENEKIGI KFCSTANNLF PSLKREKTCN |
| 481 | GRSKFNLGTQ FTAEAFLSVH ELLPMMFYYL LLTKDYSRKE |
| 521 | SADKVEGIIR KEISNIYAIY DAFANNEINS IADLTCRLQK |
| 561 | TNILQGHLPK QMISILEGRQ KDMEKEAERK IGEMIDDTQR |
| 601 | RLDLLCKQTN QKIRIGKRNA GLLKSGKIAD WLVSDMMRFQ |
| 641 | PVQKDTNNAP INNSKANSTE YRMLQHALAL FGSESSRLKA |
| 681 | YFRQMNLVGN ANPHPFLAET QWEHQTNILS FYRNYLEARK |
| 721 | KYLKGLKPQN WKQYQHFLIL KVQKTNRNTL VTGWKNSFNL |
| 761 | PRGIFTQPIR EWFEKHNNSK RIYDQILSFD RVGFVAKAIP |
| 801 | LYFAEEYKDN VQPFYDYPFN IGNKLKPQKG QFLDKKERVE |
| 841 | LWQKNKELFK NYPSEKNKTD LAYLDFLSWK KFERELRLIK |
| 881 | NQDIVTWLMF KELFKTTTVE GLKIGEIHLR DIDTNTANEE |
| 921 | SNNILNRIMP MKLPVKTYET DNKGNILKER PLATFYIEET |
| 961 | ETKVLKQGNF KVLAKDRRLN GLLSFAETTD IDLEKNPITK |
| 1001 | LSVDYELIKY QTTRISIFEM TLGLEKKLID KYSTLPTDSF |
| 1041 | RNMLERWLQC KANRPELKNY VNSLIAVRNA FSHNQYPMYD |
| 1081 | ATLFAEVKKF TLFPSVDTKK IELNIAPQLL EIVGKAIKEI |
| 1121 | EKSENKN |

Hence, the sample can be incubated with at least one CRISPR RNA (crRNA) and at least one Cas13 protein. The Cas13 protein can, for example, be a Cas13a protein, Cas13b protein, or a combination thereof.

Pre-incubation of the crRNA and Cas13 protein without the sample can facilitate RNA detection, so that the crRNA and the Cas13 protein can form a complex. For example, the Cas13 and crRNA are incubated for a period of time to form the inactive complex. In some cases, the Cas13 and crRNA complexes are formed by incubating together at 37° C. for 30 minutes, 1 hour, or 2 hours (for example, 0.5 to 2 hours) to form an inactive complex. The inactive complex can then be incubated with the reporter RNA.

### Reporter RNA

The methods and compositions described herein for detecting and/or identifying an RNA can involve incubating a mixture having a sample suspected of containing RNA, a Cas13 protein, at least one CRISPR RNA (crRNA), and a reporter RNA for a period of time to form reporter RNA cleavage products that may be present in the mixture and detecting a level of any such reporter RNA cleavage products with a detector. The detector can be a fluorescence detector.

The reporter RNA can, for example, be at least one quenched-fluorescent RNA reporter. Such quenched-fluorescent RNA reporter can optimize fluorescence detection. The quenched-fluorescent RNA reporters include an RNA oligonucleotide with both a fluorophore and a quencher of the fluorophore. The quencher decreases or eliminates the fluorescence of the fluorophore. When the Cas nuclease cleaves the RNA reporter, the fluorophore is separated from the associated quencher, such that a fluorescence signal becomes detectable.

One example of such a fluorophore quencher-labelled RNA reporter is the RNaseAlert (IDT). RNaseAlert was developed to detect RNase contaminations in a laboratory, and the substrate sequence is optimized for RNase A species. Another approach is to use lateral flow strips to detect a FAM-biotin reporter that, when cleaved by a Cas nuclease, is detected by anti-FAM antibody-gold nanoparticle conjugates on the strip. Although this allows for instrument-free detection, it requires 90-120 minutes for readout, compared to under 30 minutes for most fluorescence-based assays (Gootenberg et al. Science. 360(6387):439-44 (April 2018)).

The sequence of the reporter RNA can be optimized for Cas nuclease cleavage. Different Cas nuclease homologs can have different sequence preferences at the cleavage site. In some cases, Cas13 preferentially exerts RNase cleavage activity at exposed uridine sites or adenosine sites. There are also secondary preferences for highly active homologs.

The fluorophores used for the fluorophore quencher-labelled RNA reporters can include Alexa 430, STAR 520, Brilliant Violet 510, Brilliant Violet 605, Brilliant Violet 610, or a combination thereof.

The fluorophores used for the fluorophore quencher-labelled RNA reporters can include Dabcyl, QSY 7, QSY 9, QSY 21, QSY 35, Iowa Black Quencher (IDT), or a combination thereof. Many quencher moieties are available, for example, from ThermoFisher Scientific.

Various mechanisms and devices can be employed to detect fluorescence. Some mechanism or devices can be used to help eliminate background fluorescence. For example, reducing fluorescence from outside the detection focal plane can improve the signal-to-noise ratio, and consequently, the resolution of signal from the RNA cleavage products of interest. Total internal reflection fluorescence (TIRF) enables very low background fluorescence and single molecule sensitivity with a sufficiently sensitive camera.

In some cases, a reporter RNA can be present while the crRNA and the Cas protein form a complex. However, in other cases, the reporter RNA can be added after the crRNA and the Cas protein already form a complex. Also, after formation of the crRNA/Cas complex, the sample RNA can then be added. The sample RNA acts as an activating RNA. Once activated by the activating RNA, the crRNA/Cas complex becomes a non-specific RNase to produce RNA cleavage products that can be detected using a reporter RNA, for example, a short quenched-fluorescent RNA.

Cas13/crRNA complexes that are activated by an RNA sample cleave RNA both in cis and in trans. When cleaving in cis, for example, the activated complex can cleave the sample RNA. When cleaving in trans, the activated complex can cleave the reporter RNA, thereby releasing a signal such as the fluorophore from the reporter RNA.

### Droplets

Droplets are formed by emulsifying an aqueous reaction mixture with an oil and a surfactant to form water-in-oil droplets. Droplets containing a target RNA with the Cas nuclease/crRNA ribonucleoprotein (RNP) complex and a reporter RNA can emit fluorescence when the RNP complex binds to the target RNA.

The droplets can be formed by agitating an oil with a surfactant. The oil and surfactant are selected to provide sufficient droplet stability and to allow visualization of fluorescence within the droplets.

Droplets need not be separated from debris such as excess oil and/or surfactant prior to fluorescence monitoring. However, in some cases, background fluorescence can be reduced by separation of the droplets from the emulsion materials. A variety of methods can be used for separating the droplets from such debris. For example, the emulsion mixture can be centrifuged and the oil removed from the bottom of the tube.

To emulsify a Cas13a reaction mix, aliquots (e.g., 5-50 microliters) of an aqueous reaction mixture are combined with an excess amount of oil supplemented with a surfactant (e.g., 75-300 microliters). The oil can be HFE-7500 oil and the surfactant can be PEG-PFPE amphiphilic block copolymer surfactant (e.g., 008-Fluorosurfactant, RAN Biotechnologies). The oil can contain about 1%-5% (w/w) surfactant.

Such a reaction mixture-oil-surfactant combination can be emulsified to generate droplets ranging in diameter from at least 10 to 60 μm. In some cases, the size range is a narrower size range of about 20 to 50 μm.

The fluorescence of droplets can be directly monitored. For example, the emulsion containing the droplets can be directly loaded into a flow cell for time course imaging. In some cases, the emulsion or the separated droplets are incubated in a heating block at 37° C. before being imaged.

Although the fluorescence of droplets can be monitored in a variety of ways, in some cases the droplets are in thin layer of fluid to minimize signal overlap between overlapping droplets. A shallow flow cell can be used to minimize signal/droplet overlap. For example, such flow cells can each include two hydrophobic surfaces with sufficient space between the two surfaces for a single droplet to move about. At least one of the hydrophobic surfaces is transparent (often both are transparent) so that light can be introduced into the flow cell chamber to excite the fluorescent dye(s) of the reporter RNA, and the fluorescence emitted can be detected. The two hydrophobic surfaces can be spaced about 10μm to about 60μm apart.

For example, one hydrophobic surface of the flow cell can be an acrylic slide (75 mm×25 mm×2 mm) while the other hydrophobic surface is a siliconized coverslip (22 mm×22 mm×0.22 mm). A spacer that is about 10 μm to about 60 μm thick (e.g., about 20 μm thick) can be used to seal the edges of the coverslip to the slide. Such a flow cell can contain about 10 μl to about 60 μl fluid, where the droplets are free to move around in the fluid.

The following Examples describe some of the materials and experiments used in the develop of the invention.

### Example 1: Methods

This Example illustrates some of the materials and methods used in developing the invention.

### Protein purification

Protein purification was performed as described by Fozouni et al. (2020). Briefly, the LbauCas13a expression vector was used, which included a codon-optimized Cas13a genomic sequence, an N-terminal His6-MBP-TEV cleavage site sequence, and a T7 promoter binding sequence (Addgene Plasmid #83482). The protein was expressed in Rosetta 2 (DE3) pLysS E. coli cells in Terrific broth at 16° C. overnight. Soluble His6-MBP-TEV-Cas13a was isolated over metal ion affinity chromatography and the His6-MBP tag was cleaved with TEV protease at 4° C. overnight. Cleaved Cas13a was loaded onto a HiTrap SP column (GE Healthcare) and eluted over a linear KCl (0.25-1.0M) gradient. Cas13a-containing fractions were further purified via size-exclusion chromatography on a S200 column (GE Healthcare) in gel filtration buffer (20 mM HEPES-K pH 7.0, 200 mM KCl, 10% glycerol, 1 mM TCEP) and were subsequently flash frozen for storage at −80° C.

### Preparation of SARS-CoV-2 RNA Segments

In vitro RNA transcription was performed as described by Fozouni et al. (2020). The SARS-CoV-2 N gene, S gene (WT), and S gene with the D614G mutation were transcribed from a single-stranded DNA oligonucleotide template (IDT) using HiScribe T7 Quick High Yield RNA Synthesis Kit (NEB) following manufacturer's recommendations. Template DNA was removed by addition of DNase I (NEB), and in vitro transcribed RNA was subsequently purified using RNA STAT-60 (AMSBIO) and the Direct-Zol RNA MiniPrep Kit (Zymo Research). RNA concentration was quantified by Nanodrop and RNA copy numbers were calculated using the transcript lengths and concentrations.

### Preparation of Virus Full Genomic RNA

Full genomic viral RNAs were purified as described by Fozouni et al. (2020). Isolate USAWA1/2020 of SARS-CoV-2 (BEI Resources) was propagated in Vero CCL-81 cells. Isolate Amsterdam I of HCoV-NL63 (NR-470, BEI Resources) was propagated in Huh7.5.1-ACE2 cells. All viral cultures used in a Biosafety Level 3 laboratory. RNA was extracted from the viral supernatant via RNA STAT-60 (AMSBIO) and the Direct-Zol RNA MiniPrep Kit (Zymo Research).

crRNA Design

CRISPR RNA guides (crRNAs) were designed and validated for SARS-CoV-2. Fifteen crRNAs were first designed with 20-nt spacers corresponding to SARS-CoV-2 genome. Additional crRNAs were later designed. Each erRNA included a crRNA stem that was derived from a bacterial sequence, while the spacer sequence is derived from the SARS-CoV-2 genome (reverse complement). See Table 1A-1B (reproduced below) for examples of crRNA sequences.

TABLE 1A
Examples of SARS-COV-2 crRNA Sequences

| SEQ ID NO | Name | Sequence |
|---|---|---|
| SEQ ID NO: 1 | PF039_crLbu_nCoV_1 | GACCACCCCAAAAAUGAAGGGGACUAA |
| | (crRNA 1) | AACUUUCGCUGAUUUUGGGGUCC |
| SEQ ID NO: 2 | PF040_crLbu_nCoV_2 | GACCACCCCAAAAAUGAAGGGGACUAA |
| | (crRNA 2) | AACGGUCCACCAAACGUAAUGCG |
| SEQ ID NO: 3 | PF041_crLbu_nCoV_3 | GACCACCCCAAAAAUGAAGGGGACUAA |
| | (crRNA 3) | AACUCUGGUUACUGCCAGUUGAA |
| SEQ ID NO: 4 | PF042_crLbu_nCoV_4 | GACCACCCCAAAAAUGAAGGGGACUAA |
| | (crRNA 4) | AACUUUGCGGCCAAUGUUUGUAA |
| SEQ ID NO: 5 | PF043_crLbu_nCoV_5 | GACCACCCCAAAAAUGAAGGGGACUAA |
| | (crRNA 5) | AACGAAGCGCUGGGGGCAAAUUG |
| SEQ ID NO: 6 | PF044_crLbu_nCoV_6 | GACCACCCCAAAAAUGAAGGGGACUAA |
| | (crRNA 6) | AACAUGCGCGACAUUCCGAAGAA |
| SEQ ID NO: 7 | PF045_crLbu_nCoV_7 | GACCACCCCAAAAAUGAAGGGGACUAA |
| | (crRNA 7) | AACUUGGUGUAUUCAAGGCUCCC |
| SEQ ID NO: 8 | PF046_crLbu_nCoV_8 | GACCACCCCAAAAAUGAAGGGGACUAA |
| | (crRNA 8) | AACGGAUUGCGGGUGCCAAUGUG |
| SEQ ID NO: 9 | PF047_crLbu_nCoV_9 | GACCACCCCAAAAAUGAAGGGGACUAA |
| | (crRNA 9) | AACUGUAGCACGAUUGCAGCAUU |
| SEQ ID NO: 10 | PF048_crLbu_nCoV_10 | GACCACCCCAAAAAUGAAGGGGACUAA |
| | (crRNA 10) | AACUAAGUGUAAAACCCACAGGG |
| SEQ ID NO: 11 | PF049_crLbu_nCoV_11 | GACCACCCCAAAAAUGAAGGGGACUAA |
| | (crRNA 11) | AACUAACCUUUCCACAUACCGCA |
| SEQ ID NO: 12 | PF050_crLbu_nCoV_12 | GACCACCCCAAAAAUGAAGGGGACUAA |
| | (crRNA 12) | AACUCAGCUGAUGCACAAUCGUU |
| SEQ ID NO: 13 | PF051_crLbu_nCoV_13 | GACCACCCCAAAAAUGAAGGGGACUAA |
| | (crRNA 13) | AACUCUAGCAGGAGAAGUUCCCC |
| SEQ ID NO: 14 | PF052_crLbu_nCoV_14 | GACCACCCCAAAAAUGAAGGGGACUAA |
| | (crRNA 14) | AACUCUGUCAAGCAGCAGCAAAG |
| SEQ ID NO: 15 | PF053_crLbu_nCoV_15 | GACCACCCCAAAAAUGAAGGGGACUAA |
| | (crRNA 15) | AACCUUUGCUGCUGCUUGACAGA |
| SEQ ID NO: 16 | PF083_crLbu_nCov12v2 | GACCACCCCAAAAAUGAAGGGGACUAA |
| | | AACAACGAUUGUGCAUCAGCUGA |
| SEQ ID NO: 17 | PF084_crLbu_nCov15v2 | GACCACCCCAAAAAUGAAGGGGACUAA |
| | | AACGACAUUUUGCUCUCAAGCUG |
| SEQ ID NO: 18 | PF085_crLbu_nCoV_16 | GACCACCCCAAAAAUGAAGGGGACUAA |
| | (crRNA 16) | AACGUUCCUGGUCCCCAAAAUUU |
| SEQ ID NO: 19 | PF086_crLbu_nCoV_17 | GACCACCCCAAAAAUGAAGGGGACUAA |
| | (crRNA 17) | AACUGGCACCUGUGUAGGUCAAC |
| SEQ ID NO: 20 | PF087_crLbu_nCoV_18 | GACCACCCCAAAAAUGAAGGGGACUAA |
| | (crRNA 18) | AACUCCAUGCCAAUGCGCGACAU |
| SEQ ID NO: 21 | PF088_crLbu_nCoV_19 | GACCACCCCAAAAAUGAAGGGGACUAA |
| | (crRNA 19) | AACCUAUUAACUAUUAACGUACC |
| SEQ ID NO: 22 | PF089_crLbu_nCoV_20 | GACCACCCCAAAAAUGAAGGGGACUAA |
| | (crRNA 20) | AACUAUUGCAGCAGUACGCACAC |
| SEQ ID NO: 23 | PF090_crLbu_nCoV_21 | GACCACCCCAAAAAUGAAGGGGACUAA |
| | (crRNA 21) | AACAGCGCAGUAAGGAUGGCUAG |
| SEQ ID NO: 24 | PF091_crLbu_nCoV_22 | GACCACCCCAAAAAUGAAGGGGACUAA |
| | (crRNA 22) | AACGUAACUAGCAAGAAUACCAC |
| SEQ ID NO: 25 | PF092_crLbu_nCov_2XL | UAGACCACCCCAAAAAUGAAGGGGACU |
| | (crRNA 2XL) | AAAACGGUCCACCAAACGUAAUGCG |
| SEQ ID NO: 26 | PF093 crLbu_nCov_4XL | UAGACCACCCCAAAAAUGAAGGGGACU |
| | (crRNA 4XL) | AAAACGGUCCACCAAACGUAAUGCG |
| SEQ ID NO: 27 | cr2 (one of the 8G | uagaccaccccaaaaaugaaggggacuaaaacCGCAUU |
| | crRNAs) detecting protein | ACGUUUGGUGGACC |
| | N | |
| | Lower case: stem | |
| | sequence | |
| | Upper case: Target | |
| | sequence | |
| SEQ ID NO: 28 | Cr4 (one of the 8G | uagaccaccccaaaaaugaaggggacuaaaacUUACAA |
| | crRNAs) detecting protein | ACAUUGGCCGCAAA |
| | N | |
| | Lower case: stem | |
| | sequence | |
| | Upper case: Target | |
| | sequence | |
| SEQ ID NO: 29 | NCR_542 (one of the 8G | uagaccaccccaaaaaugaaggggacuaaaacAAACUA |
| | crRNAs) detecting | CGUCAUCAAGCCAA |
| | ORF1ab (NSP5) | |
| | Lower case: stem | |
| | sequence | |
| | Upper case: Target | |
| | sequence | |
| SEQ ID NO: 30 | NCR_546 (one of the 8G | uagaccaccccaaaaaugaaggggacuaaaacCACAGU |
| | crRNAs) detecting | CAUAAUCUAUGUUA |
| | ORF1ab (NSP5) | |
| | Lower case: stem | |
| | sequence | |
| | Upper case: Target | |
| | sequence | |
| SEQ ID NO: 31 | NCR_564 (one of the 8G | uagaccaccccaaaaaugaaggggacuaaaacUCACAC |
| | crRNAs) detecting | UUUUCUAAUAGCAU |
| | ORF1ab (NSP16) | |
| | Lower case: stem | |
| | sequence | |
| | Upper case: Target | |
| | sequence | |
| SEQ ID NO: 32 | NCR_569 (one of the 8G | uagaccaccccaaaaaugaaggggacuaaaacUGUAAG |
| | crRNAs) detecting the S | AUUAACACACUGAC |
| | protein | |
| | Lower case: stem | |
| | sequence | |
| | Upper case: Target | |
| | sequence | |
| SEQ ID NO: 33 | NCR_588 (one of the 8G | uagaccaccccaaaaaugaaggggacuaaaacUUAAUU |
| | crRNAs) detecting protein | GUGUACAAAAACUG |
| | N | |
| | Lower case: stem | |
| | sequence | |
| | Upper case: Target | |
| | sequence | |
| SEQ ID NO: 34 | NCR_596 (one of the 8G | uagaccaccccaaaaaugaaggggacuaaaacCAGUUG |
| | crRNAs) detecting protein | UGAUGAUUCCUAAG |
| | ORF8 | |
| | Lower case; stem | |
| | sequence | |
| | Upper case: Target | |
| | sequence | |
| SEQ ID NO: 35 | Guide 21 detecting | uagaccaccccaaaaaugaaggggacuaaaacAGCGCA |
| | protein E | GUAAGGAUGGCUAG |
| SEQ ID NO: 36 | crRNA 11A | UAGACCACCCCAAAAAUGAAGGGGACU |
| | | AAAACGCUUGUGUUACAUUGUAUGC |
| SEQ ID NO: 37 | crRNA 12A | UAGACCACCCCAAAAAUGAAGGGGACU |
| | | AAAACAAUUUGAUGGCACCUGUGUA |

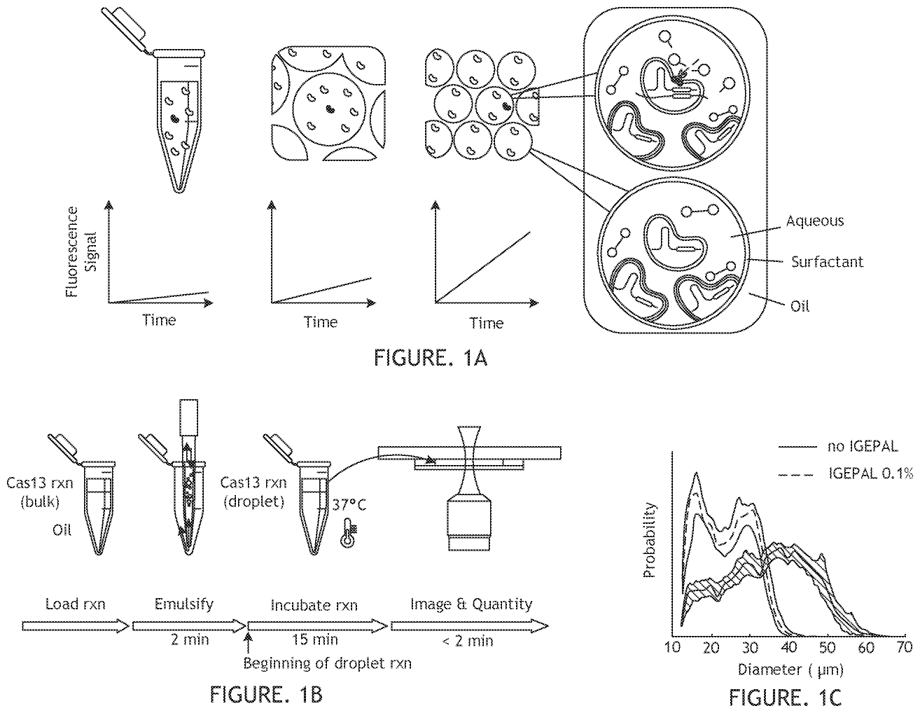

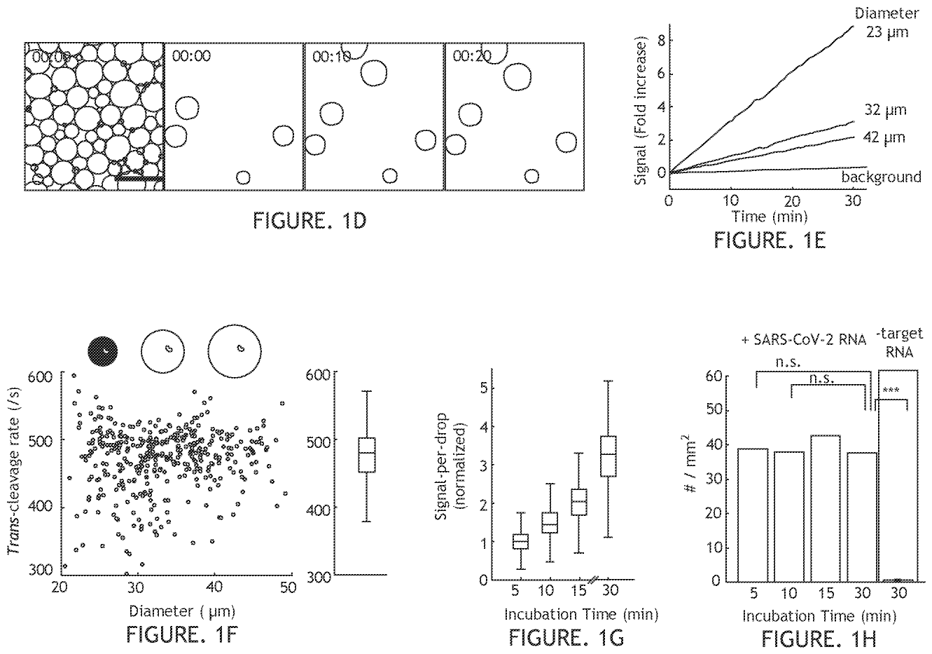

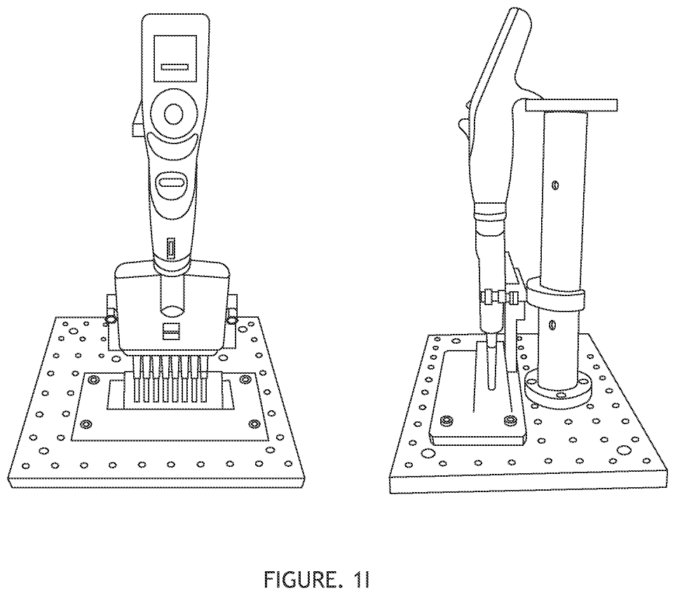

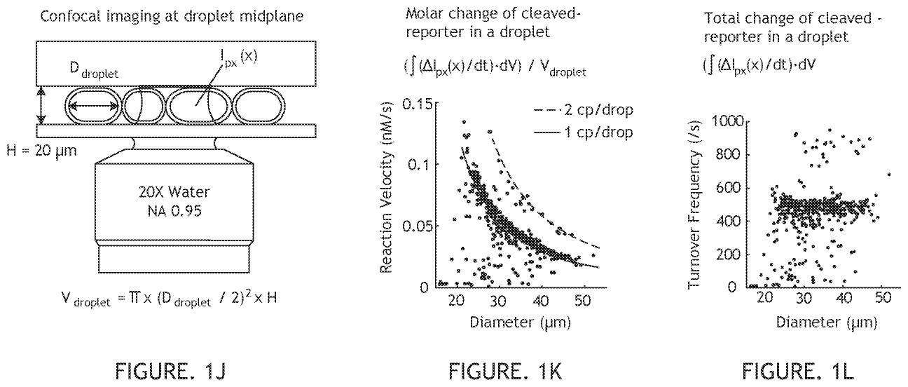

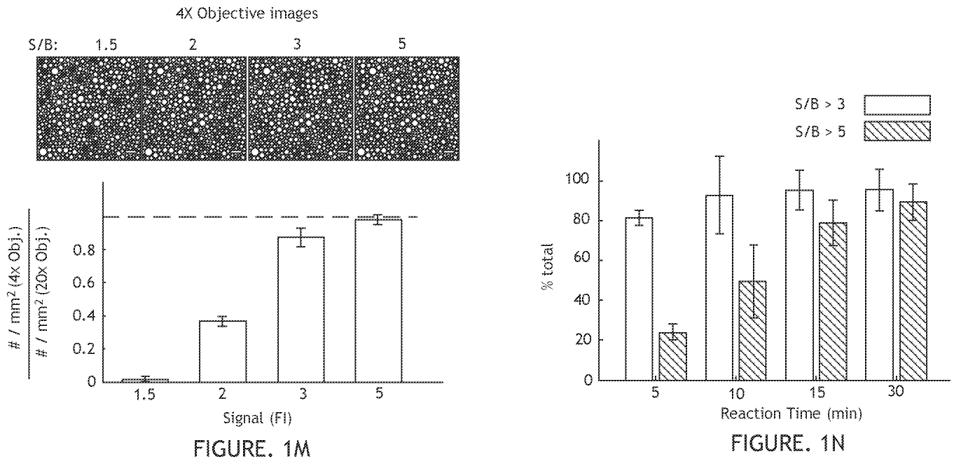

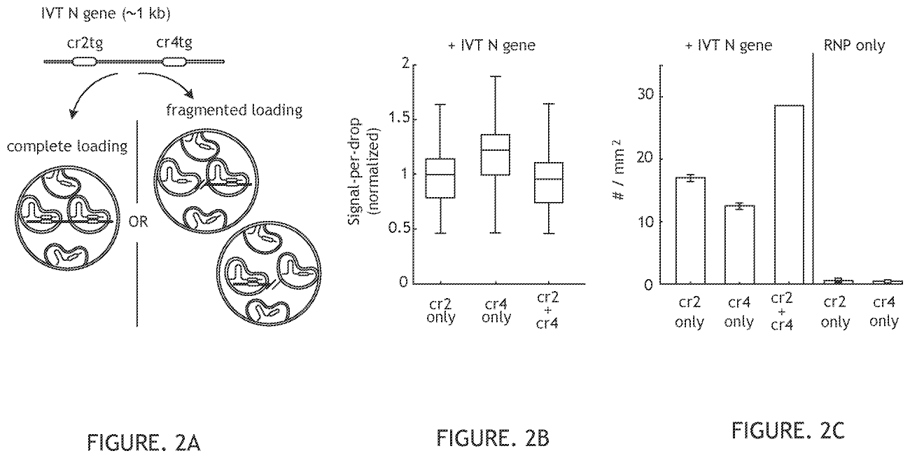

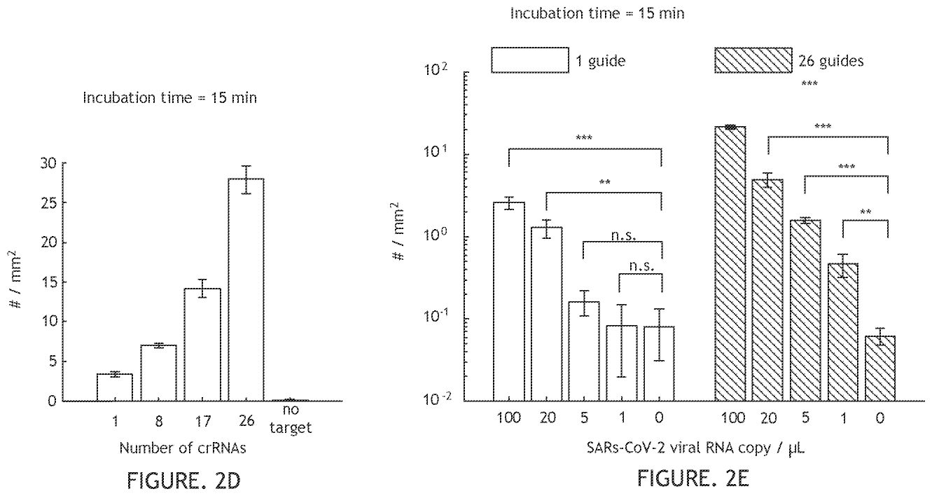

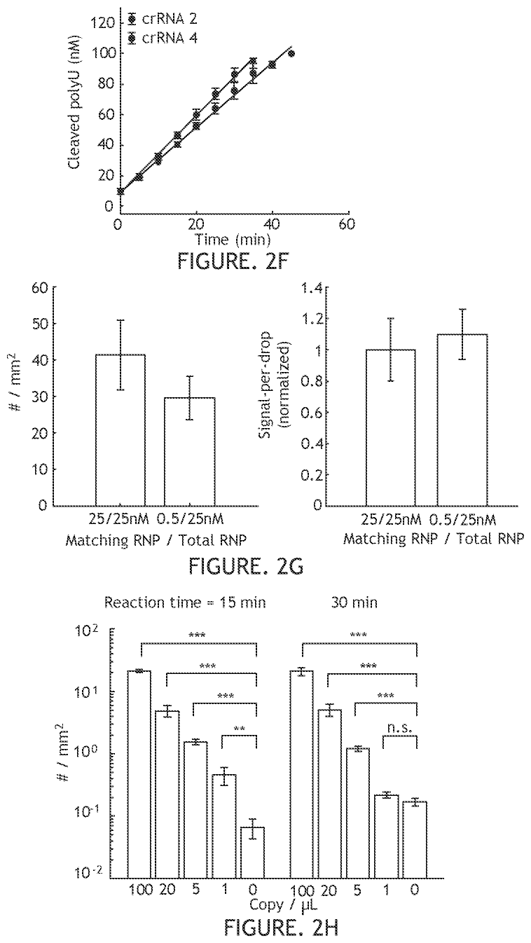

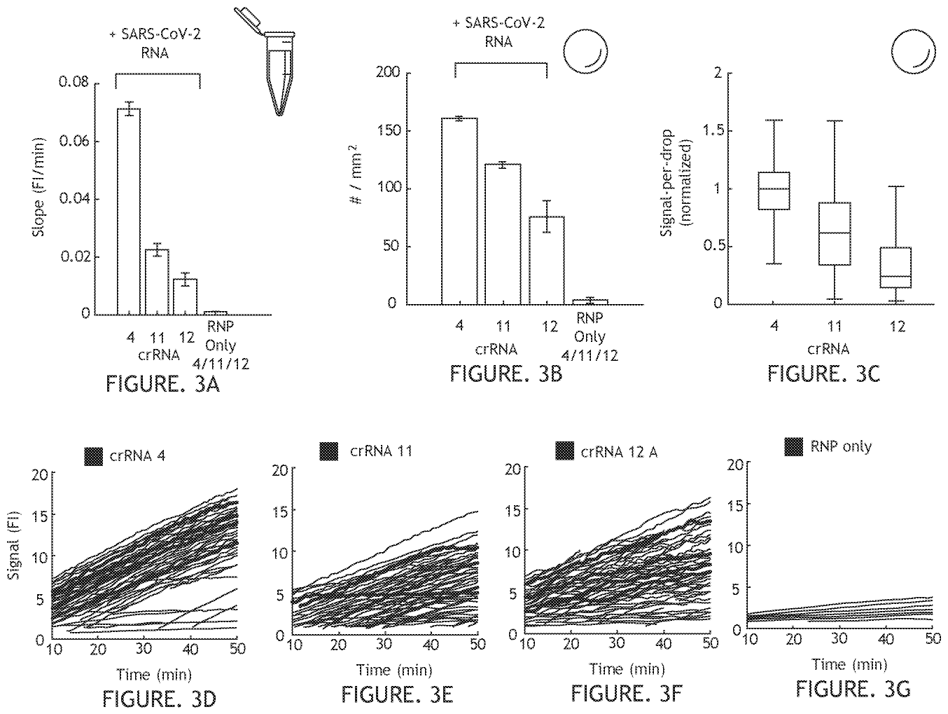

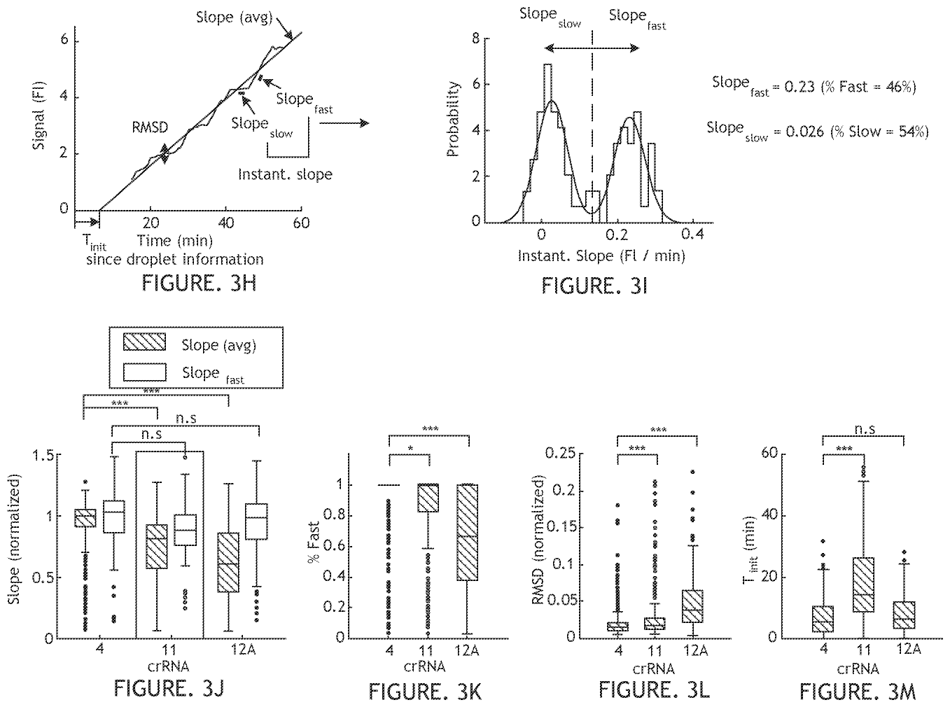

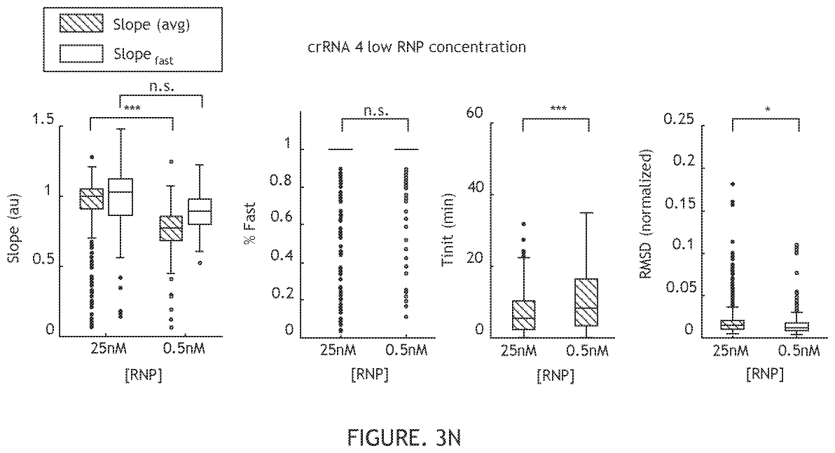

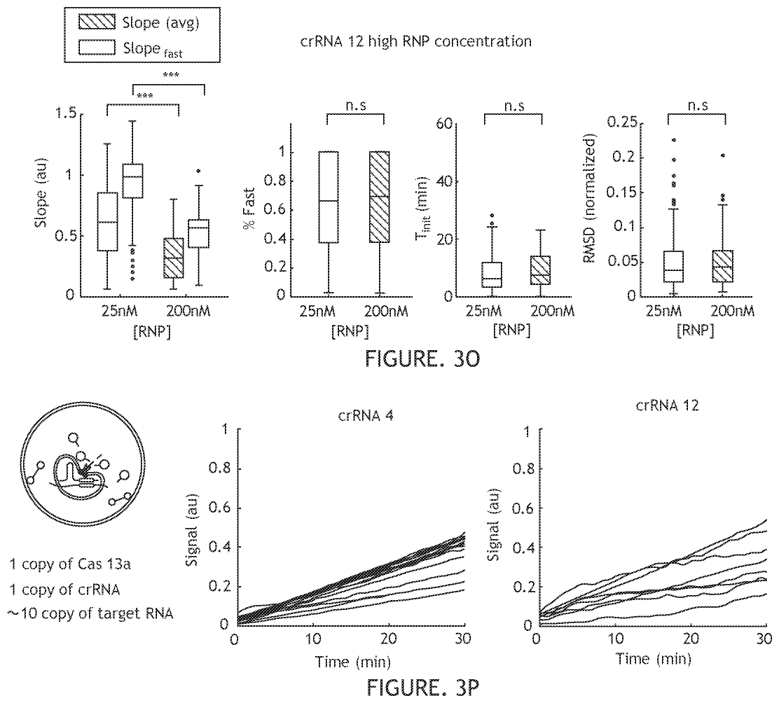

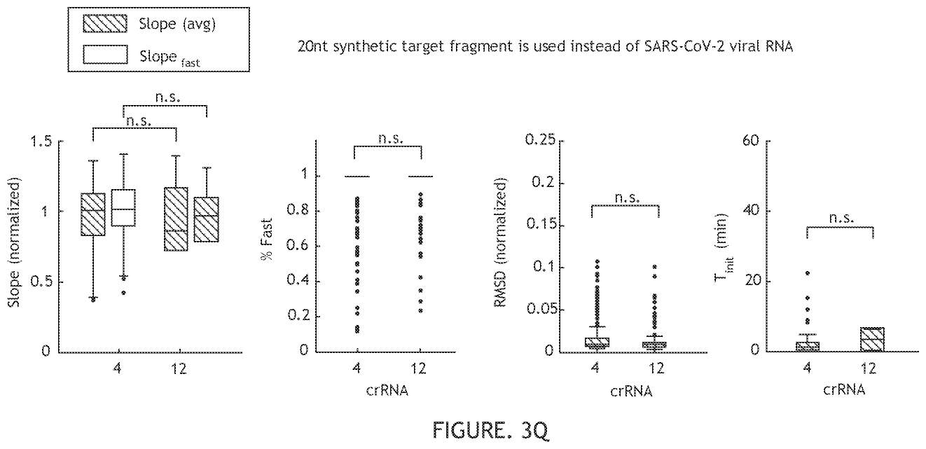

TABLE 1B
crRNAs used to Generate the Data in the Figures

| | | | Combination |
|---|---|---|---|
| Name | Sequence | Used in | in FIG. 2D |
| crRNA 2 | GACCACCCCAAAAAUGAAGGGGACU | FIG. 2B-2E, 2H | 8, 17, 26 |
| | AAAACGGUCCACCAAACGUAAUGOG | | |
| | (SEQ ID NO: 2) | | |
| crRNA 4 | GACCACCCCAAAAAUGAAGGGGACU | FIG. 1D-I1N, 2B-2H, | 1, 8, 17, 26 |
| | AAAACUUUGCOGCCAAUGUUUGUAA | 3A-3C, 3D, 3J-M, | |
| | (SEQ ID NO: 4) | 2A-C, 3A, 3C, 3D | |
| crRNA | UAGACCACCCCAAAAAUGAAGGGGACU | FIG. 3A-C, 3E, 3J-M | — |
| 11A | AAAACGCUUGUGUUACAUUGUAUGC | | |
| | (SEQ ID NO: 36) | | |
| crRNA | UAGACCACCCCAAAAAUGAAGGGGACU | FIG. 3A-C, 3F, 3H- | 26 |
| 12A | AAAACAAUUUGAUGGCACCUGUGUA | 3M, 30-3Q 4C-4E. | |
| | (SEQ ID NO: 37) | | |
| crRNA D3 | UAGACCACCCCAAAAAUGAAGGGGACU | FIG. 2D-2E, 2H | 8, 17, 26 |
| | AAAACAAACUACGUCAUCAAGCCAA | | |
| | (SEQ ID NO: 38) | | |
| crRNA D7 | UAGACCACCCCAAAAAUGAAGGGGACU | FIG. 2D-2E, 2H | 8, 17, 26 |
| | AAAACCACAGUCAUAAUCUAUGUUA | | |
| | (SEQ ID NO: 39) | | |
| crRNA F1 | UAGACCACCCCAAAAAUGAAGGGGACU | FIG. 2D-2E, 2H | 8, 17, 26 |
| | AAAACUCACACUUUUCUAAUAGCAU | | |
| | (SEQ ID NO: 40) | | |
| crRNA F6 | UAGACCACCCCAAAAAUGAAGGGGACU | FIG. 2D-2E, 2H | 8, 17, 26 |
| | AAAACUGUAAGAUUAACACACUGAC | | |
| | (SEQ ID NO: 41) | | |
| crRNA H1 | UAGACCACCCCAAAAAUGAAGGGGACU | FIG. 2D-2E, 2H | 8, 17, 26 |
| | AAAACUUAAUUGUGUACAAAAACUG | | |
| | (SEQ ID NO: 42) | | |
| crRNA H9 | UAGACCACCCCAAAAAUGAAGGGGACU | FIG. 2D-2E, 2H | 8, 17, 26 |
| | AAAACCAGUUGUGAUGAUUCCUAAG | | |
| | SEQ ID NO: 43) | | |
| crRNA A1 | UAGACCACCCCAAAAAUGAAGGGGACU | FIG. 2D-2E, 2H | 17, 26 |
| | AAAACUGAUAAGACCUCCUCCACGG | | |
| | (SEQ ID NO: 44) | | |
| crRNA B1 | UAGACCACCCCAAAAAUGAAGGGGACU | FIG. 2D-2E, 2H | 17, 26 |
| | AAAACCAAAAGCCUCAACACGUAGA | | |
| | (SEQ ID NO: 45) | | |
| crRNA B2 | UAGACCACCCCAAAAAUGAAGGGGACU | FIG. 2D-2E, 2H | 17, 26 |
| | AAAACCAUGUUGAAACAAGUAACUC | | |
| | (SEQ ID NO: 46) | | |
| crRNA C1 | UAGACCACCCCAAAAAUGAAGGGGACU | FIG. 2D-2E, 2H | 17, 26 |
| | AAAACUCAACGAUGUAAGAAGACUG | | |
| | (SEQ ID NO: 47) | | |
| crRNA C2 | UAGACCACCCCAAAAAUGAAGGGGACU | FIG. 2D-2E, 2H | 17, 26 |
| | AAAACACACUAUCAACGAUGUAAGA | | |
| | (SEQ ID NO: 48) | | |
| crRNA C8 | UAGACCACCCCAAAAAUGAAGGGGACU | FIG. 2D-2E, 2H | 17, 26 |
| | AAAACUAACCAUCCACUGAAUAUGU | | |
| | (SEQ ID NO: 49) | | |
| crRNA H2 | UAGACCACCCCAAAAAUGAAGGGGACU | FIG. 2D-2B, 2H | 17, 26 |
| | AAAACAAACAGUAAGGCCGUUAAAC | | |
| | (SEQ ID NO: 50) | | |
| crRNA H3 | UAGACCACCCCAAAAAUGAAGGGGACU | FIG. 2D-2E, 2H | 17, 26 |
| | AAAACUCCAAUUGUGAAGAUUCUCA | | |
| | (SEQ ID NO: 51) | | |
| crRNA H5 | UAGACCACCCCAAAAAUGAAGGGGACU | FIG. 2D-2E, 2H | 17, 26 |
| | AAAACGACUAAAGCAUAAAGAUAGA | | |
| | (SEQ ID NO: 52) | | |
| crRNA B3 | UAGACCACCCCAAAAAUGAAGGGGACU | FIG. 2D-2B, 2H | 26 |
| | AAAACAAAAUUAUCGAAGCUUGCGU | | |
| | (SEQ ID NO: 53) | | |
| crRNA B8 | UAGACCACCCCAAAAAUGAAGGGGACU | FIG. 2D-2E, 2H | 26 |
| | AAAACCAGUACAUCAAACGAAUUUG | | |
| | (SEQ ID NO: 54) | | |
| crRNA C12 | UAGACCACCCCAAAAAUGAAGGGGACU | FIG. 2D-2E, 2H | 26 |
| | AAAACGGAAAUACAAAUGAUAUAAG | | |
| | (SEQ ID NO: 55) | | |
| crRNA E12 | UAGACCACCCCAAAAAUGAAGGGGACU | FIG. 2D-2E, 2H | 26 |
| | AAAACAAUUUCCAUUUGACUCCUGG | | |
| | (SEQ ID NO: 56) | | |
| crRNA E4 | UAGACCACCCCAAAAAUGAAGGGGACU | FIG. 2D-2E, 2H | 26 |
| | AAAACCAAUCUUUAAAGAGUCCUGU | | |
| | (SEQ ID NO: 57) | | |
| crRNA E7 | UAGACCACCCCAAAAAUGAAGGGGACU | FIG. 2D-2E, 2H | 26 |
| | AAAACUUAAAUUGAUCUCCAGGOGG | | |
| | (SEQ ID NO: 58) | | |
| crRNA H6 | UAGACCACCCCAAAAAUGAAGGGGACU | FIG. 2D-2E, 2H | 26 |
| | AAAACGAAGUAGACUAAAGCAUAAA | | |
| | (SEQ ID NO: 59) | | |
| crRNA H8 | UAGACCACCCCAAAAAUGAAGGGGACU | FIG. 2D-2E, 2H | 26 |
| | AAAACCACCGGUGAUCCAAUUUAUU | | |
| | (SEQ ID NO: 60) | | |
| HCoV | GACCACCCCAAAAAUGAAGGGGACUAA | FIG. 4C-4E, 4L | — |
| crRNA 63 | AACCCUCUCUGGUAGGAACACGC (SEQ ID | | |
| | NO: 61) | | |
| crRNA 6 | GACCACCCCAAAAAUGAAGGGGACUAA | FIG. 2D-2E, 2H | — |
| | AACAAGAUCCUGAUAAAGAACAG (SEQ ID | | |
| | NO:62) | | |
| crRNA 45 | GACCACCCCAAAAAUGAAGGGGACUAA | FIG. 4I, 4N | — |
| | AACCUUUCCAUACAACUUUUGUU (SEQ ID | | |
| | NO: 63) | | |
| crRNA 21 | GACCACCCCAAAAAUGAAGGGGACUAAA | FIG. 2G | |
| | ACAGCGCAGUAAGGAUGGCUAG (SEQ ID | | |
| | NO: 23) | | |
| crRNA4A | GCACAGCAGAAAATCTCTGCUUACAAAC | | |
| 20 nt target | AUUGGCCGCAAACCACAG¹ | | |
| | (SEQ ID NO: 64) | | |
| orRNA | GCACAGCAGAAAATCTCTGCUACACAGG | | |
| 12C | UGCCAUCAAAUUCCACAG¹ | | |
| 20 nt target | (SEQ ID NO: 65) | | |
| PolyU | /56-FAM/rUrUrUrUrU/3IABKFQ/ | FIG. 3A, FIG. 2F | |
| Reporter | (SEQ ID NO: 65) | | |
| PolyU | /Alexa488/rUrUrUrUrU/3IABKFQ/ | All figures | |
| Reporter | (SEQ ID NO: 66) | | |
| PF025_ | CCACCCCAAAAAUGAAGGGGACUAAAAC | | |
| crLbu_ NT1 | aGGUUCUUGACUACCGUAAUU (SEQ ID | | |
| non- | NO: 67) | | |
| targeting | | | |
| PF026_ | CCACCCCAAAAAUGAAGGGGACUAAAAC | | |
| crLbu_NT2 | aUCGGAUGUAAAUUAUGCCGU (SEQ ID | | |
| non- | NO: 68) | | |
| targeting | | | |
| PF027_ | CCACCCCAAAAAUGAAGGGGACUAAAAC | | |
| crLbu_NT3 | aCAGCUCAUCGGUGUCCUACU (SEQ ID | | |
| non- | NO: 69) | | |
| targeting | | | |
| PF099_ | GACCACCCCAAAAAUGAAGGGGACUAAA | | |
| crLbu_NT1_M | ACGGUUCUUGACUACCGUAAUU (SEQ ID | | |
| non- | NO: 70) | | |
| targeting | | | |

¹The oligonucleotide is consisted of DNA (underlined) followed by RNA. The 20-nt oligonucleotides complementary to the crRNA spacer sequence is indicated in bold.

### Bulk Cas13a Nuclease Assays

LbuCas13a-crRNA RNP complexes were first preassembled at 133 nM equimolar concentrations for 15 minutes at room temperature and then diluted to 25 nM LbuCas13a in cleavage buffer (20 mM HEPES-Na pH 6.8, 50 mM KCl, 5 mM MgCl2, and 5% glycerol) in the presence of 400 nM of reporter RNA (5′-Alexa488rUrUrUrUrU-IowaBlack FQ-3′), 1 U/μL Murine RNase Inhibitor (NEB, Cat #M0314), 0.1 vol % IGEPAL 630 (Fisher, Cat #ICN 19859650), and varying amounts of target RNA. For reactions using more than one crRNA, multiple guides were combined at equal concentrations and subsequently the total crRNA mix was assembled with Cas13 at 133 nM equimolar concentration. Twenty-five nM (25 nM) of RNP complex were used unless specified otherwise. The reaction mix was measured either in bulk or as droplets following emulsification (see droplet formation). For the bulk Cas13a assay, the reaction mix was loaded into a 0.2 mL eight-tube strip (Fisher Cat #14-222-251) and incubated in a compact fluorescence detector (Axxin, T16-ISO) for 1 hour at 37° C. with fluorescence measurements taken every about 30 seconds (FAM channel, gain 20). Fluorescence values were normalized by the values obtained from reactions containing only reporter and buffer.

### Droplet Formation

To emulsify a Cas13a reaction mix, 20 μL of an aqueous mix was combined with 100 μL of HFE-7500 oil supplemented with 2% (w/w) PEG-PFPE amphiphilic block copolymer surfactant (008-Fluorosurfactant, RAN Biotechnologies) in a 0.2 mL eight tube-strip. The oil/aqueous mix was emulsified by repeated pipetting without any manual handling using an electronic 8-channel pipette (Integra biosciences, Part #4623) with a 200 μL pipet tip (VWR Cat #37001-532). The electronic pipette was used to mix 110 μL of sample volume for 150 repetitions at the maximum speed (speed 10) to emulsify droplets to a narrow size range. The emulsion was either directly loaded into a flow cell for time course imaging or incubated in a heating block at 37° C. before being transferred and imaged. In both cases, the emulsion was quickly separated by spinning in a speed-controlled mini-centrifuge (about 50 rpm) for 10 seconds, the oil was completely removed from the bottom of the tube, and the emulsion was transferred into a custom flow cell after several cycles of gentle manual mixing.

### Flow Cell for Droplet Imaging

The sample flow cell was prepared by sandwiching double-sided tape (about 20 μm thick, 3M Cat #9457) between an acrylic slide (75 mm×25 mm×2 mm, laser cut from a 2 mm-thick acrylic plate) and a siliconized coverslip (22 mm×22 mm×0.22 mm, Hampton research Cat #500829). Both surfaces were hydrophobic, promoting thin layers of oil between the droplets and the two surfaces. Siliconized coverslips were rinsed with isopropanol to remove any auto-fluorescent debris (20 minutes sonication) and spin dried prior to assembly. Fifteen microliters of sample emulsion was loaded into the flow cell by capillary action, after which the inlet and outlet were sealed with Valap sealant.

### Microscopy and Data Acquisition

Droplet imaging was carried out on an inverted Nikon Eclipse Ti microscope (Nikon Instruments) equipped with a Yokogawa CSU-X spinning disk. A 488-nm solid state laser (ILE-400 multimode fiber with BCU, Andor Technologies) was used to excite the RNA fluorescent probe. The fluorescence light was spectrally filtered with an emission 535/40 nm filter (Chroma Technology) and imaged using an sCMOS camera (Zyla 4.2, Andor Technologies). A 20× water-immersion objective (CFI Apo LWD Lambda S, NA 0.95) was used with the Perfect Focus System to monitor droplets during the course of reaction and/or to accurately quantify fluorescence signals at reaction endpoints. Images were acquired through Micro-Manager under X W/cm² 488-nm excitation with 500 ms exposure time and 2×2 camera binning. Typically, sixteen field-of-views (FOV) are acquired every 30 seconds for the time course of imaging and 36 field-of views were acquired for the endpoint imaging. A 4× objective (CFI Plan Apo Lambda, NA 0.20) was used for the high-throughput droplet imaging at reaction endpoints. Thirty-six FOVs were acquired under controlled excitation with 3 second exposure time without camera binning.

### Image Analysis—Droplet Detection

A custom MATLAB (Mathworks R2020b) script was used to detect positive droplets and quantify fluorescence signals. First, the grayscale images were converted to binary images based on a locally adaptive threshold. The threshold was defined generously at this stage to select all the positive droplets and potentially some negative droplets or debris. Second, connected droplets were separated by watershed transform. Third, individual droplets were identified by looking for circular continuous regions and droplet parameters such as radius, circularity. The fluorescence signals were then quantified in two different ways: the mean fluorescence signal of a droplet reflecting the density of cleaved reporter; and the total fluorescence signal reflecting the total amount of cleaved reporter within a droplet. Lastly, positive droplets were chosen based on their circularity and total fluorescence signal by applying a threshold that were consistently used throughout the experiments.

### Image Analysis—Droplet Tracking in Time Course Images

To quantify signal accumulation in the same droplet over time, droplets were associated with their motion over time as estimated by a Kalman filter in MATLAB. The filter was used to predict the track's location in each frame and to determine the likelihood of each detection within a frame being assigned to a particular track. Only the droplets showing continuous trajectories in time and magnitude are selected for downstream analysis.

### Comparison of Single Cas13a Reaction With Enzyme Kinetics

The Cas13a reaction was analyzed with a single crRNA (FIG. 1F) using the Michaelis Menten enzyme kinetics model with the quasi-steady-state approximation:

$$v = \frac{Kcat [ {E}_{0} ] [ S ]}{{K}_{M} + [ S ]}$$

where ν is the reaction rate, [E₀] is ternary Cas13a, [S] is the RNA reporter, K_{cat} and K_{M} are the catalytic rate constant and the Michaelis constant. When low substrate concentration was used ([S]<<K_{M}), because the RNA reporter [S] was 400 nM and K_{M} was estimated to be larger than 1 μM (Slaymaker et al., 2019) the equation simplified to:

$$v = \frac{Kcat [ {E}_{0} ] [ S ]}{{K}_{M}}$$

where ν/[E₀] was turnover frequency, or the reciprocal of the mean waiting time <1/t>in the single molecule Michaelis-Menten framework (Min et al., 2005). The ν/[E₀] turnover frequency could be obtained from FIG. 1K-1L after converting the fluorescence signal to molar concentration of cleaved reporter based on a calibration.

### Data Analysis—Cas13a Time Trajectories

The raw signal was processed in a series of steps prior to analysis.

First, the raw signal was corrected for the global signal fluctuation, which arises from a slight drift in z-focus even with the Perfect Focus System. The global signal was characterized from the background droplets and was identified from the histogram of pixel values. In particular, the global signal was divided from the positive droplet signal in each image frame.

Second, the inventors corrected for the photobleaching. The signal decay rate was characterized from more than 200 Cas13a curves exhibiting negative slopes and positive initial signals. Photobleaching was modeled as a linear function of initial signal based on the observed linear relationship between the decay rate versus the initial signal (R²=0.87). Using this model, each trajectory point-by-point was corrected for photobleaching.

Third, the trajectories were filtered with a weak Savitzky-Golay filter (order 5, frame length 9) to remove the high frequency measurement noise while preserving overall structure of the curve.

Lastly, instantaneous slopes were calculated by dividing signal changes between frames by the frame interval and removing single outliers exhibiting high positive or negative slopes.

To characterize key parameters of Cas13 kinetics, individual trajectories were analyzed in two different domains. First, the slope, time from target addition to the initiation of enzyme activity (Tinit), and RMSD were determined from signal time trajectories by linear regression. Because Tinit indicates time since droplet reaction, a constant time (12.5 minutes) was added that reflected the time from Cas13 droplet formation until the beginning of time course imaging. Second, the slopefast, slopeslow, and a fraction spent in each period were determined by fitting a gaussian pdf to the instantaneous slope distribution. The model qualities were compared between the single versus binary gaussian pdfs using Akaike's Information Criterion (AIC) to determine whether a trajectory exhibits two different periods of slope of not.

### Data Analysis—Kinetic Barcoding

The slope and RMSD of individual signal trajectories were used to compare Cas13a reactions between different target-crRNAs. Binary classification of trajectories was first performed based on the Supported Vector Machine (SVM) in MATLAB. For this, 200 to 400 signal trajectories in each condition we collected, and two or more independent experiments per condition were performed to prevent bias. The trajectories were converted into a 2D array consisting of the slope and RMSD and the array was divided into a training and a validation set. An algorithm was then trained using the training set with the known answers (i.e. known target-crRNA conditions) and the validation set was classified. The accuracy of identifying individual trajectories was 75% for HCoV-NL63 RNA versus SARS-CoV-2 RNA, and 73% for wild type versus D614G RNA (the D614G RNA was from a SARS-CoV-2 strain having a D614G mutation in its Spike protein). To access significance between two groups of trajectories, a two-tailed Student's t-test was employed to the predicted class and reported p-values.

### Example 2: High Sensitivity, High Specificity Multiplex RNA Detection

This Example demonstrates that RNA detection with high sensitivity and multiplexed specificity can be achieved despite short detection times by encapsulating the Cas13 reaction in droplets and monitoring enzyme kinetics fluorescently. The methods described herein enable quantification of the absolute amount of target RNA based on the number of positive droplets. However, the small droplet volume employed accelerates signal accumulation of the direct Cas13 reaction. When a single target RNA is encapsulated in a droplet with a volume of approximately 10 picoliters as illustrated in FIG. 1A, the Cas13 signal accumulation rate is equivalent to that of a bulk reaction containing 10⁵ copies/μL of target RNA.

To rapidly generate millions of droplets with volumes of about 10 μL, reaction mixtures containing LbuCas13a were emulsified in an excess volume of an oil/surfactant/detergent mixture as described in Example 1. The resulting droplets were imaged on an inverted fluorescence microscope (FIG. 1B, 1I-1J). Millions of droplets ranging from 10 to 40 μm diameter were formed after 2 minutes of pipetting with an automatic multi-channel pipettor (FIG. 1C, 1I). Imaging the droplets allowed normalization of the fluorescence signal by droplet size (Byrnes et al., 2018) and avoided the need for slower and more complex systems to generate uniform droplet sizes.

The Cas13 droplet assay was validated by forming droplets containing 10,000 copies/μL of SARS-CoV-2 RNA, along with LbuCas13a, crRNA targeting the SARS-CoV-2 N gene (crRNA 4, SEQ ID NO: 4) and a fluorophore-quencher pair tethered by RNA (reporter), and monitoring the reaction of positive droplets over time (FIG. 1D). At this target concentration, about 7% droplets contain the target RNA, with the vast majority of those containing only a single copy.

The signal accumulation rate in droplets was inversely proportional to droplet size (FIG. 1K), with smaller droplets increasing faster than larger droplets. As shown in FIG. 1E, a 9-fold increase in signal was observed for 23 μm droplets compared to a 3-fold increase in signal for 42 μm droplets.

Measurements showed that a single LbuCas13a can cleave 471±47 copies of reporter every second in the presence of 400 nM reporter, indicating that the K_{cat}/K_{M} is 1.2×10⁹ M⁻¹s⁻¹, which is two orders-of-magnitude higher than that measured for LbCas12a (Chen et al., 2018). These results are also consistent with those measured for LbuCas13a based on a bulk assay (Shan et al., 2019). Notably, the absolute trans-cleavage rate of a single LbuCas13 remains consistent regardless of droplet size (FIG. 1F).

Longer incubation times resulted in linear increases in the average signals per droplet (FIG. 1G). All the positive reactions could be correctly identified in reaction times as little as five minutes with a 20X/0.95NA objective (FIG. 1H) and 15 minutes using a 4×/0.20NA microscope objective (FIG. 1M-1N; S/B refers to signal over background).

Guide combinations were tested to determine whether more signal could be obtained per target RNA and whether the detection time would be reduced in the Cas13a droplet assays. In vitro transcribed (IVT) target RNA corresponding to the N gene of SARS-CoV-2 (nucleotide positions 28274-29531) was used in droplets containing crRNA 2 (SEQ ID NO:2), or crRNA 4 (SEQ ID NO:4), or both crRNAs as illustrated in FIG. 2A. The number of the positive droplets and the quantity of signal from the positive droplets was measured.

Surprisingly, although crRNAs 2 and 4 generated similar signals when used individually (FIG. 2F) and might be expected to double the signal when both are present, the signal per droplet was not significantly different when using two crRNAs than when using just one crRNA (FIG. 2B). Instead, the number of positive droplets almost doubled when droplets contained both crRNA 2 and crRNA 4 compared to just one of the crRNAs (FIG. 2C). These data indicate that the N gene in vitro transcribed RNA was fragmented through cis-cleavage upon initiation of the reaction prior to droplet formation, causing the regions targeted by different crRNAs to be loaded into separate droplets (FIG. 2A). Hence, use of multiple crRNAs activates independent Cas13a reactions in different droplets.

Increased guide combinations were evaluated in the droplet assay mixtures to ascertain whether they affect the number of detectable (positive) droplets. In initial experiments, twenty-six crRNAs were made by the inventors that targeted different regions of SARSCOV-2 genome, and that individually produced strong Cas13 signals (Table 1A).

As shown in FIG. 2D, adding additional types of crRNAs while keeping the total RNP concentration to 25 nM, increased the number of positive droplets. The activity of Cas13a (signal per drop) remained constant even when only a small fraction of the total RNPs in a droplet contained crRNA matching the target (FIG. 2G). These data indicate that a large number of crRNAs (e.g., 50 or more) can be combined to maximize the number of independent Cas13a reactions when targeting the same target RNA.

Droplet-based assays are fundamentally limited by the false-positive rate in the absence of target reactions, hence the generation of multiple positive droplets per target RNA can increase sensitivity of the assay.

To further evaluate the sensitivity of the Cas13a droplet assay with guide combinations, serial dilutions were made of precisely tittered SARS-CoV-2 genomic RNA obtained from the Biodefense and Emerging Infections Research Resources Repository (BEI Resources). The number of positive droplets in each dilution was quantified using either a single crRNA or all 26 crRNAs, using thirty-six images per condition (˜160,000 droplets) after 15 minutes of reaction incubation (FIG. 2E). For the single crRNA (crRNA 4, SEQ ID NO:4), the number of positive droplets remained significantly higher than the no-target control for the samples containing twenty (20) target copies/μL or more (FIG. 2E). For the combination of twenty-six (SEQ ID NO:1-26) crRNAs, the direct detection limit of detection was lower than 1 copy/μL target, comparable to the sensitivity of PCR. This limit of detection was not improved if the assay reaction was incubated for 30 minutes instead of 15 minutes (FIG. 2H).

The fast Cas13a kinetics achievable in droplets depended on the crRNA and its target. For example, as illustrated in FIG. 3A, two crRNAs targeting a different segment of SARS-CoV-2 N gene., crRNA 11A and crRNA 12A (SEQ ID NOs:36 and 37), exhibited significantly slower rates in a bulk reaction than crRNA 2 or 4 (SEQ ID NO:2 or 4). For this reason, selection of crRNAs that support efficient Cas13 activity is important for Cas13-based molecular diagnostics, though how different guide crRNAs affect the activity of Cas13 is not well understood (Wessels et al., 2020).

The droplet assay was compared to bulk assays while evaluating Cas13a enzymatic activity in the presence of single guide crRNAs (and hence, single targets were detected in these experiments). As shown in FIG. 3B, while the number of positive droplets was reduced for guide crRNA 11A and 12A (SEQ ID NO:36 and 37) compared to crRNA 4 (SEQ ID NO:4), the reduction in droplet count was significantly less than the change observed in bulk reaction (compare FIG. 3B with FIG. 3A). On the other hand, as shown in FIG. 3C, the signal in each positive droplet was significantly reduced for both erRNA 11A and 12A (SEQ ID NO:36 and 37), compared to crRNA 4 (SEQ ID NO:4).

To further understand these differences, individual reaction trajectories were examined within positive droplets, where the reaction trajectories were reported as the change in fluorescence for each 30 second measurement time period. Interestingly, individual erRNA:Cas13a assays exhibited rich kinetic behaviors that were crRNA-dependent. As shown in FIG. 3D-3F, different endpoint signals resulted when using different guide crRNAs. The slope, shape, and x-intercept of the individual trajectories varied widely in droplets depending on the crRNA. In some cases, the trajectories exhibited striking stochastic behaviors, exhibiting periods of no signal increase followed by periods of rapid signal increase. However, the majority of positive droplets observed for all three crRNAs exhibited slopes significantly higher (FIGS. 3D-3F) than the rare positive slopes exhibited for populations of RNP-only droplets (FIG. 3G). These data indicate that the specific combination of crRNA and target significantly affects Cas13a enzymatic activity even when a single target RNA is present.

To quantify differences in Cas13a kinetics within droplets, individual signal trajectories were characterized by their average slope, root-mean-square-deviation (RMSD), and time from target addition to the initiation of enzyme activity (Tinit) (FIG. 3H). By calculating the instantaneous slopes at each point in the trajectory and fitting the slope distribution to a Gaussian curve, the inventors found that the trajectories exhibiting two different slopes: one ‘fast’ when fluorescence is increasing and one ‘slow’ when fluorescence is not increasing (FIG. 3I). Interestingly, even though the average slopes differed significantly for the different crRNAs (SEQ ID NO:4, 36, and 37), the instantaneous ‘fast’ slopes were relatively constant across all three crRNAs (FIG. 3J). Consistent with this, crRNA 12A (SEQ ID NO:37), which exhibited the lowest average slope of the three guide crRNAs, exhibited extended slow periods (FIG. 3K) and increased levels of signal fluctuation (FIG. 3L). In addition, the Tinit varied significantly among the three crRNAs evaluated. For example, crRNA 11A (SEQ ID NO:36) exhibited the slowest Tinit of all (FIG. 3M), resulting in reduced signal at the reaction endpoint (FIG. 3C).

To test if the stochastic behavior of the Cas13a reaction was caused by the unbinding of crRNA from Cas13a, the RNP concentration was changed to be either below or above the Kd of crRNA-Cas13a. However, the stochastic behaviors remained unaltered (FIG. 3N-3O) despite the changes in RNP concentration. In fact, the kinetic features remain qualitatively the same for both crRNA 4 (SEQ ID NO:4) and crRNA 12A (SEQ ID NO:37) even for droplets containing only single copies of each of the three Cas13a components: the Cas13a, the crRNA, and the target (FIG. 3P). In contrast, when the SARS-CoV-2 RNA target was replaced with a 20-nucleotide fragment complementary to the crRNA12A spacer sequence (i.e. crRNA12C; SEQ ID NO:65), the stochastic behavior of the reaction was no longer observed and Tinit was significantly shortened (FIG. 3Q). These data indicate that the reduced Cas13a activity observed for crRNA 12A (SEQ ID NO:37) was caused by the target RNA-its sequence, local folding characteristics, or global folding characteristics.

Based on the distinct kinetic signatures observed for the different crRNA and target combinations, the inventors hypothesized that specific crRNA-target pairs could be identified based on their signal trajectories. As illustrated in FIG. 4A-4B, the distinct crRNA:target kinetic signatures observed provide a method for multiplexed detection of different RNA viruses or different virus variants in a single droplet when using one fluorescent reporter. To test this hypothesis, referred to herein as ‘kinetic barcoding,’ a crRNA was first combined with a common cold virus NL-63 (crRNA 63; SEQ ID NO:61) and a second crRNA targeting SARS-CoV-2 (crRNA 12A; SEQ ID NO:37). These two crRNA were chosen because they individually exhibit different kinetic signatures (FIG. 4C). Thirty-minute trajectories were collected from hundreds of droplets containing either NL63 or SARS-CoV-2 RNA. The droplets also contained Cas13a and both crRNAs. The two groups of trajectories were clearly distinguishable based on their average slopes and Root-mean-square-deviation (RMSD) (FIG. 4D).

To determine how clearly the NL63 RNA and the SARS-CoV-2 RNA can be distinguished, a subset of trajectories was randomly sampled and their differences were compared by performing Student's t-test on their binary classification result using the methods described in Example 1 (see FIG. 4E). Increasing the number of trajectories and extending the measurement time improved classification (FIG. 4K), though measurement times longer than 10 minutes did not provide any improvement.

Overall, these data indicate that NL-63 and SARS-CoV-2 can be distinguished within 10 minutes provided that 20 or more trajectories are measured. Similar results are achieved when images were acquired every 3 minutes for 30 minutes instead of every 30 seconds for 10 minutes (FIG. 4L).

Next, the kinetic barcoding methods were evaluated to determine whether a mutant viral strain could be differentiated from the wild-type strain. One crRNA was used that targeted the variable region of SARS-CoV-2 S-protein and the signal trajectories generated from the in vitro transcribed wild type S gene were compared to the trajectories from the in vitro transcribed S gene harboring the D614G mutation. The D614G mutation is shared by all SARS-CoV-2 variants (CDC, 2020). As shown in FIG. 4F, although both wild type and mutant signal trajectories are smooth (i.e. they exhibit low RMSD), the average slopes obtained with the mutant target were significantly lower than that of WT (see also FIG. 4G). Using the difference in the average slopes of 30 or more signal trajectories, the D614G mutant RNA could be distinguished from the wild type RNA within 5 minutes (FIG. 4M).

The California SARS-CoV-2 variant (B.1.427/B.1.429; Epsilon) was tested using the kinetic barcoding method to confirm its utility when using a clinical sample. The California SARS-CoV-2 variant (B.1.427/B.1.429) harbors a unique S13I mutation and exhibits increased transmissibility and reduced neutralization by convalescent and post-vaccination sera (CDC, 2020). A crRNA targeting the region encompassing S13I mutation in SARS-CoV-2 S-protein was used that matched the mutant sequence. Viral RNA extracted from cultured viruses as well as RNA from patient samples was evaluated, where the RNA was known to have either the wild type or the B. 1.427 sequence. The patient samples exhibited Ct values of 15 to 20 in PCR testing and provided 15 to 350 positive trajectories among the droplets measured. Although individual trajectories from each sample exhibited heterogenous slopes and RMSDs, the slopes measured from the WT were significantly lower than those measured from the B.1.427 mutant (FIGS. 4I and 4N).

To test if the B.1.427/B.1.429 mutant strain could be correctly identified when only 10 individual trajectories are collected, 10 trajectories were randomly evaluated from each sample. As shown in FIG. 4I-4J, regardless of the choice of 10 trajectories, the average of slope distribution clearly distinguished between the WT and B. 1.427 RNA, with a detection accuracy of about 99%.

Overall, the data described herein demonstrate that a droplet-based Cas13 direct detection assay can achieve PCR-level sensitivity and can simultaneously distinguish different RNA targets based on their reaction kinetics. Because a crRNA can be diluted by 50 times or more without compromising its performance in the droplet-based assay, many different types of crRNAs can used within a droplet to further enhance detection sensitivity to lower than 1 copies/μL. At this sensitivity, the Cas13a direct detection droplet assay can be used in situations where extremely low viral loads are present. For example, the droplet cases Cas assay can be used for environmental samples, cancer miRNAs, latent HIV virus, as well as for different SARS-CoV-2 variants without the limitations and potential loss of RNA due to sample purification, reverse transcription, or amplification.

The LbuCas13 was also found to be an efficient, diffusion-limited enzyme whose kinetics are controlled by the specific combination of crRNA and the target. The distribution of single Cas13 RNP's activity was homogenous for crRNAs supporting high activities (FIG. 1F), suggesting that the active conformation of Cas13a RNP is stable over time. However, we found that certain crRNAs can switch off Cas13a activity for more than a minute. The observation that the stochastic signal is reduced when a short RNA fragment is presented instead of the full virus RNA (FIG. 3Q) indicates that the target RNA's local or global structure plays a role in the kinetics of Cas13a RNP. The data indicated that the effects of enzyme conformational switching (Liu et al., 2017) do not play a significant role.

On the other hand, RNA mismatches between a crRNA and its target can reduce the slope of reaction without introducing the stochastic activity switching (FIG. 4G-4H), indicating that multiple mechanisms can result in diverse Cas13a kinetics. Based on those kinetic signatures, the droplet methods were able to determine which virus or variant was present in a given droplet.

Digital assays are useful at enhancing the sensitivity and quantitative performance in ddPCR (Hindson et al., 2013; McDermott et al., 2013), protein detection (Rissin et al., 2010), and recently CRISPR-Cas-based nucleic acid detection (Ackerman et al., 2020; Shinoda et al., 2021; Tian et al., 2021; Yue et al., 2021). While some detection assays use existing ddPCR technologies, amplification-free Cas13a assays require smaller droplets (about 10μL) than ddPCR (about 900μL (Pinheiro et al., 2012)) to achieve useful signal amplification.

The droplet-based Cas13a direct detection assay with kinetic barcoding described herein enable rapid and sensitive molecular diagnostics for multiple RNA viruses and RNA biomarkers.

### Example 3: Programmable Kinetic Barcoding With crRNA-DNA Hybrids

After demonstrating the feasibility of kinetic barcoding based on natural differences in kinetics of crRNA and target RNA, the inventors developed an improved programmable way to control the kinetic signature of a crRNA, independent of its target RNA.

The inventors hypothesized that when a DNA fragment is added proximal to Cas13a's HEPN site, it will constantly interfere with its trans-cleavage activity for RNA without being digested, thus slowing down the rate of reporter RNA cleavage. To test this, the inventors added a DNA fragment of varying sequence and length to the S′-end of crRNA 4 (SEQ IN NO:4) to form a DNA-crRNA, which can reach to the HEPN site every time the crRNA is loaded to Cas13a (FIG. 5A). The DNA-crRNA was thus divided into an effector region, the sequence of which can alter Cas13a's nuclease activity, and an 8bp-long linker region that connects the effector DNA and crRNA.

The reporter signal was measured in a droplet containing either the DNA-crRNA 4 or unmodified crRNA 4 (SEQ ID NO:4) along with a single SARS-CoV-2 RNA target at the assay endpoint. As shown in FIG. 5B, Cas13's trans-cleavage rate was reduced for DNA-crRNA 4, with longer DNA and thymine (T) nucleotides rather than adenine (A) nucleotides in its effector region showing stronger reductions. These results indicate that increasing the local concentration of DNA near the HEPN site (by adding more nucleotides in the 5′-end of crRNA) or addition of DNA sequences matching the trans-cleavage preference (by adding more thymine repeats) will interfere with its nuclease activity more strongly. As shown in FIG. 5C, the number of positive droplets decreased only slightly when a DNA-crRNA 4 hybrid is used instead of crRNA 4 (SEQ ID NO:4). Therefore, such crRNA tuning can work for any target sequence and enables precise tuning of Cas13a kinetics at the single-molecule level without compromising its ability to recognize target.

This kinetic barcoding strategy was then tested to evaluate whether it can improve multiplexed virus detection. Four different crRNAs were selected that target different virus RNAs but provide identical trans-cleavage rate for its respective target (FIG. 5D-5E) (crRNA 4 for SARS-CoV-2 wildtype, crRNA delta for SARS-CoV-2 delta, crRNA NL63 for HCoV-NL-63, and crRNA H3N2 for H3N2 influenza virus). A DNA fragment of varying sequence was then added a to each crRNA (FIG. 5E). The 1-hour signal trajectories were then measured from droplets containing individual target virus RNA. As shown in FIG. 5D, the signal intensities per drop were similar, and as shown in FIG. 5F, the signal trajectories were linear. In addition, the signal slopes are clearly separated from one virus to another (FIG. 5E-5F). As illustrated in FIG. 5E, each viral target had a distinct normalized signal slope. The normalized signal slope for H3N2 influenza virus (IAV H3N2) ranged from about 0.2 to 0.4 (peak at about 0.3); the normalized signal slope for SARS-CoV-2 wildtype (SC2 WT) ranged from about 0.3 to 0.5 (peak at about 0.4); the normalized signal slope for SARS-CoV-2 delta (SC2 delta) ranged from about 0.4 to 0.7 (peak at about 0.58); and the normalized signal slope for HCoV-NL-63 (NL-63) ranged from about 0.6 to 0.8 (peak at about 0.7).

Importantly, the slopes for each virus remained the same even when all four crRNAs are combined into the same droplet (FIG. 5G), making it possible to simultaneously detect different targets based on their unique slopes. There were two signal peaks for SARS-CoV-2 delta when the crRNAs were combined because both crRNA 4 and crRNA delta targeted the SARS-CoV-2 delta RNA.

In other experiments, the inventors focused on the three viruses that exhibited a single signal peak when using the crRNA-combination to classify new samples containing either one or two different viruses based on the slope of the signals. As shown in FIG. 5H, the kinetic barcoding methods and assay mixtures not only correctly identified the virus target but also quantified the proportion of each infection among the possible single or dual infection scenarios.

This Example therefore illustrates a crRNA modification that enables precise tuning of LbuCas13's trans-cleavage rate towards the reporter RNA. Simultaneous detection of different SARS-CoV-2 variants was achieved in clinical samples. This kinetic barcoding approach, which works based on the strict RNA-preference of LbuCas13a's nuclease activity, will work with other Cas13 orthologs as well as with other CRISPR-Cas systems. When multiple Cas13 orthologs and other CRIPSR-Cas systems are combined with kinetic barcoding, more than ten target pathogens could be readily detected.

### Example 4: A Single Kinetic Barcoding Measurement Accurately Differentiate Virus Variants

This Example illustrates that the kinetic barcoding assay methods work simply by detecting the droplet signal at the assay endpoint instead of monitoring its time trajectory.

This simplifies the the application of kinetic barcoding. This is plausible because our new kinetic barcoding strategy only alters reaction slope without introducing any stochasticity to it. To test this capability, the two dominant SARS-CoV-2 variants circulating at the time of study (SARS-CoV-2 delta and omicron) were evaluated using the kinetic barcoding method to ascertain whether they could be differentiated from wildtype SARS-CoV-2.

In addition to crRNA 4 targeting a conserved region of SARS-CoV-2 RNA, two crRNAs were used that are specific to unique mutations in delta (crRNA delta) or omicron (crRNA omicron) and added DNA modifications (FIG. 6). Using the automated assay workflow and the low magnification imaging (2.5×/NA0.6), each viral target was mixed with the three-crRNA combination and the droplet signal was measured after incubation for 1 hour.

As shown in FIG. 6, the wildtype virus RNA exhibited a single peak distribution while the delta or omicron RNA exhibited two peaks-one corresponding to the variant and the other to the shared region of SARS-CoV-2 genome.

### REFERENCES

Ackerman, C. M., Myhrvold, C., Thakku, S. G., Freije, C. A., Metsky, H. C., Yang, D. K., Ye, S. H., Boehm, C. K., Kosoko-Thoroddsen, T .-S. F., Kehe, J., Nguyen, T. G., Carter, A., Kulesa, A., Barnes, J. R., Dugan, V. G., Hung, D. T., Blainey, P. C., Sabeti, P. C., 2020. Massively multiplexed nucleic acid detection with Cas13. Nature 1-6. doi.org/10.1038/s41586-020-2279-8
Bar-Even, A., Noor, E., Savir, Y., Liebermeister, W., Davidi, D., Tawfik, D. S., Milo, R., 2011. The Moderately Efficient Enzyme: Evolutionary and Physicochemical Trends Shaping Enzyme Parameters. Biochemistry 50, 4402-4410. doi.org/10.1021/bi2002289
Byrnes, S. A., Chang, T. C., Huynh, T., Astashkina, A., Weigl, B. H., Nichols, K. P., 2018. Simple Polydisperse Droplet Emulsion Polymerase Chain Reaction with Statistical Volumetric Correction Compared with Microfluidic Droplet Digital Polymerase Chain Reaction. Anal. Chem. 90, 9374-9380. doi.org/10.1021/acs.analchem.8b01988
CDC, 2020. Coronavirus Disease 2019 (COVID-19) Cent. Dis. Control Prev. See website: cdc.gov/coronavirus/2019-ncov/variants/variant-info.html (accessed 6.2.21).
Chen, J. S., Ma, E., Harrington, L. B., Costa, M. D., Tian, X., Palefsky, J. M., Doudna, J. A., 2018. CRISPR-Cas12a target binding unleashes indiscriminate single-stranded DNase activity. Science 360, 436-439. doi.org/10.1126/science.aar6245
East-Seletsky, A., O'Connell, M. R., Knight, S. C., Burstein, D., Cate, J. H. D., Tjian, R., Doudna, J. A., 2016. Two distinct RNase activities of CRISPR-C2c2 enable guide-RNA processing and RNA detection. Nature 538, 270-273. doi.org/10.1038/nature19802
Fozouni, P., Son, S., Derby, M. D. de L., Knott, G. J., Gray, C. N., D'Ambrosio, M. V., Zhao, C., Switz, N. A., Kumar, G. R., Stephens, S. I., Boehm, D., Tsou, C .-L., Shu, J., Bhuiya, A., Armstrong, M., Harris, A. R., Chen, P .-Y., Osterloh, J. M., Meyer-Franke, A., Joehnk, B., Walcott, K., Sil, A., Langelier, C., Pollard, K. S., Crawford, E. D., Puschnik, A. S., Phelps, M., Kistler, A., DeRisi, J. L., Doudna, J. A., Fletcher, D. A., Ott, M., 2020. Amplification-free detection of SARS-CoV-2 with CRISPR-Cas13a and mobile phone microscopy. Cell 0. doi.org/10.1016/j.cell.2020.12.001
Fozouni, P., Son, S., Diaz de León Derby, M., Knott, G. J., Gray, C. N., D'Ambrosio, M. V., Zhao, C., Switz, N. A., Kumar, G. R., Stephens, S. I., Boehm, D., Tsou, C.-L., Shu, J., Bhuiya, A., Armstrong, M., Harris, A. R., Chen, P .-Y., Osterloh, J. M., Meyer-Franke, A., Joehnk, B., Walcott, K., Sil, A., Langelier, C., Pollard, K. S., Crawford, E. D., Puschnik, A. S., Phelps, M., Kistler, A., DeRisi, J. L., Doudna, J. A., Fletcher, D. A., Ott, M., 2021. Amplification-free detection of SARS-CoV-2 with CRISPR-Cas13a and mobile phone microscopy. Cell 184, 323-333.e9. doi.org/10.1016/j.cell.2020.12.001
Gootenberg, J. S., Abudayyeh, O. O., Lee, J. W., Essletzbichler, P., Dy, A. J., Joung, J., Verdine, V., Donghia, N., Daringer, N. M., Freije, C. A., Myhrvold, C., Bhattacharyya, R. P., Livny, J., Regev, A., Koonin, E. V., Hung, D. T., Sabeti, P. C., Collins, J. J., Zhang, F., 2017. Nucleic acid detection with CRISPR-Cas13a/C2c2. Science 356, 438-442. doi.org/10.1126/science.aam9321
Heid, C. A., Stevens, J., Livak, K. J., Williams, P. M., 1996. Real time quantitative PCR. Genome Res. 6, 986-994. doi.org/10.1101/gr.6.10.986
Hindson, B. J., Ness, K. D., Masquelier, D. A., Belgrader, P., Heredia, N. J., Makarewicz, A. J., Bright, I. J., Lucero, M. Y., Hiddessen, A. L., Legler, T. C., Kitano, T. K., Hodel, M. R., Petersen, J. F., Wyatt, P. W., Steenblock, E. R., Shah, P. H., Bousse, L. J., Troup, C. B., Mellen, J. C., Wittmann, D. K., Erndt, N. G., Cauley, T. H., Koehler, R. T., So, A. P., Dube, S., Rose, K. A., Montesclaros, L., Wang, S., Stumbo, D. P., Hodges, S. P., Romine, S., Milanovich, F. P., White, H. E., Regan, J. F., Karlin-Neumann, G. A., Hindson, C. M., Saxonov, S., Colston, B. W., 2011. High-Throughput Droplet Digital PCR System for Absolute Quantitation of DNA Copy Number. Anal. Chem. 83, 8604-8610. doi.org/10.1021/ac202028g

Hindson, C. M., Chevillet, J. R., Briggs, H. A., Gallichotte, E. N., Ruf, I. K., Hindson, B. J., Vessella, R. L., Tewari, M., 2013. Absolute quantification by droplet digital PCR versus analog realtime PCR. Nat. Methods 10, 1003-1005. https://doi.org/10.1038/nmeth.2633
Jiao, C., Sharma, S., Dugar, G., Peeck, N. L., Bischler, T., Wimmer, F., Yu, Y., Barquist, L., Schoen, C., Kurzai, O., Sharma, C. M., Beisel, C. L., 2021. Noncanonical crRNAs derived from host transcripts enable multiplexable RNA detection by Cas9. Science. doi.org/10.1126/science.abe7106
Liu, L., Li, X., Ma, J., Li, Z., You, L., Wang, J., Wang, M., Zhang, X., Wang, Y., 2017. The Molecular Architecture for RNA-Guided RNA Cleavage by Cas13a. Cell 170, 714-726.e10. doi.org/10.1016/j.cell.2017.06.050
Liu, T. Y., Knott, G. J., Smock, D. C. J., Desmarais, J. J., Son, S., Bhuiya, A., Jakhanwal, S., Prywes, N., Agrawal, S., Derby, M. D. de L., Switz, N. A., Armstrong, M., Harris, A. R., Charles, E. J., Thornton, B. W., Fozouni, P., Shu, J., Stephens, S. I., Kumar, G. R., Zhao, C., Mok, A., Iavarone, A. T., Escajeda, A. M., McIntosh, R., Kim, S. E., Dugan, E. J., Consortium, I. T., Pollard, K. S., Tan, M. X., Ott, M., Fletcher, D. A., Lareau, L. F., Hsu, P. D., Savage, D. F., Doudna, J. A., 2021. Accelerated RNA detection using tandem CRISPR nucleases. medRxiv 2021.03.19.21253328. doi.org/10.1101/2021.03.19.21253328
McDermott, G. P., Do, D., Litterst, C. M., Maar, D., Hindson, C. M., Steenblock, E. R., Legler, T. C., Jouvenot, Y., Marrs, S. H., Bemis, A., Shah, P., Wong, J., Wang, S., Sally, D., Javier, L., Dinio, T., Han, C., Brackbill, T. P., Hodges, S. P., Ling. Y., Klitgord, N., Carman, G. J., Berman, J. R., Koehler, R. T., Hiddessen, A. L., Walse, P., Bousse, L., Tzonev, S., Hefner, E., Hindson, B. J., Cauly, T. H., Hamby, K., Patel, V. P., Regan, J. F., Wyatt, P. W., Karlin-Neumann, G. A., Stumbo, D. P., Lowe, A. J., 2013. Multiplexed Target Detection Using DNA-Binding Dye Chemistry in Droplet Digital PCR. Anal. Chem. 85, 11619-11627. doi.org/10.1021/ac403061n
Metsky, H. C., Welch, N. L., Haradhvala, N. J., Rumker, L., Zhang, Y. B., Pillai, P. P., Yang, D. K., Ackerman, C. M., Weller, J., Blainey, P. C., Myhrvold, C., Mitzenmacher, M., Sabeti, P. C., 2021. Diagnostic design with machine learning model-based optimization. bioRxiv 2020.11.28.401877. doi.org/10.1101/2020.11.28.401877
Min, W., English, B. P., Luo, G., Cherayil, B. J., Kou, S. C., Xie, X. S., 2005. Fluctuating Enzymes: Lessons from Single-Molecule Studies. Acc. Chem. Res. 38, 923-931. doi.org/10.1021/ar040133f
Pinheiro, L. B., Coleman, V. A., Hindson, C. M., Herrmann, J., Hindson, B. J., Bhat, S., Emslie, K. R., 2012. Evaluation of a Droplet Digital Polymerase Chain Reaction Format for DNA Copy Number Quantification. Anal. Chem. 84, 1003-1011. doi.org/10.1021/ac202578x
Rissin, D. M., Kan, C. W., Campbell, T. G., Howes, S. C., Fournier, D. R., Song, L., Piech, T., Patel, P. P., Chang, L., Rivnak, A. J., Ferrell, E. P., Randall, J. D., Provuncher, G. K., Walt, D. R., Duffy, D. C., 2010. Single-molecule enzyme-linked immunosorbent assay detects serum proteins at subfemtomolar concentrations. Nat. Biotechnol. 28, 595-599. doi.org/10.1038/nbt.1641
Samson, R., Deutch, J. M., 1978. Diffusion-controlled reaction rate to a buried active site. J. Chem. Phys. 68, 285-290. doi.org/10.1063/1.435494
Shan, Y., Zhou, X., Huang, R., Xing, D., 2019. High-Fidelity and Rapid Quantification of miRNA Combining crRNA Programmability and CRISPR/Cas13a trans-Cleavage Activity. Anal. Chem. 91, 5278-5285. doi.org/10.1021/acs.analchem. 9b00073
Shinoda, H., Taguchi, Y., Nakagawa, R., Makino, A., Okazaki, S., Nakano, M., Muramoto, Y., Takahashi, C., Takahashi, I., Ando, J., Noda, T., Nureki, O., Nishimasu, H., Watanabe, R., 2021. Amplification-free RNA detection with CRISPR-Cas13. Commun. Biol. 4, 1-7. doi.org/10.1038/s42003-021-02001-8
Slaymaker, I. M., Mesa, P., Kellner, M. J., Kannan, S., Brignole, E., Koob, J., Feliciano, P.R., Stella, S., Abudayyeh, O. O., Gootenberg, J. S., Strecker, J., Montoya, G., Zhang, F., 2019. High-Resolution Structure of Cas13b and Biochemical Characterization of RNA Targeting and Cleavage. Cell Rep. 26, 3741-3751.e5. doi.org/10.1016/j.celrep.2019.02.094
Tambe, A., East-Seletsky, A., Knott, G. J., Doudna, J. A., O'Connell, M. R., 2018. RNA Binding and HEPN-Nuclease Activation Are Decoupled in CRISPR-Cas13a. Cell Rep. 24, 1025-1036. doi.org/10.1016/j.celrep.2018.06.105
Tian, T., Shu, B., Jiang, Y., Ye, M., Liu, L., Guo, Z., Han, Z., Wang, Z., Zhou, X., 2021. An Ultralocalized Cas13a Assay Enables Universal and Nucleic Acid Amplification-Free Single-Molecule RNA Diagnostics. ACS Nano 15, 1167-1178. doi.org/10.1021/acsnano.0c08165
Wessels, H .-H., Mendez-Mancilla, A., Guo, X., Legut, M., Daniloski, Z., Sanjana, N. E., 2020. Massively parallel Cas13 screens reveal principles for guide RNA design. Nat. Biotechnol. 38, 722-727. doi.org/10.1038/s41587-020-0456-9
Yue, H., Shu, B., Tian, T., Xiong, E., Huang, M., Zhu, D., Sun, J., Liu, Q., Wang, S., Li, Y., Zhou, X., 2021. Droplet Cas12a Assay Enables DNA Quantification from
Unamplified Samples at the Single-Molecule Level. Nano Lett. doi.org/10.1021/acs.nanolett.1c00715

All publications, patent applications, patents and other references mentioned herein are expressly incorporated by reference in their entirety, to the same extent as if each were incorporated by reference individually. In case of conflict, the present specification, including definitions, will control.

The following statements provide a summary of some aspects of the inventive nucleic acids and methods described herein.

### Statements

1 An assay mixture comprising a population of droplets ranging in diameter from at least 10 to 60 μm, the population of droplets comprising a test droplet subpopulation comprising at least one ribonucleoprotein complex, plus at least one reporter RNA, plus at least one target RNA.
2. The assay mixture of statement 1, wherein the at least one ribonucleoprotein complex comprises at least one Cas nuclease and at least one CRISPR guide RNA (crRNA).
3. The assay mixture of statement 2, wherein at least one of the CRISPR guide RNAs (crRNAs) comprises or consists essentially of an RNA-polymer hybrid, wherein a polymer is covalently linked to the 5′-end of at least one crRNA.
4. The assay mixture of statement 3, wherein the polymer inhibits cleavage of at least one of the reporter RNAs.
5. The assay mixture of statement 3 or 4, wherein the polymer reduces activity or folding of the Cas nuclease higher-eukaryotes-and-prokaryotes nucleotide-binding (HEPN) domain.
6. The assay mixture of any one of statements 3-5, wherein the polymer comprises polyethylene glycol, poly[oxy(11-(3-(9-adeninyl)propionato)-undecanyl-1-thiomethyl)ethylene] (PECH-AP), poly[oxy(11-(5-(9-adenylethyloxy)-4-oxopentanoato)undecanyl-1-thiomethyl)ethylene] (PECH-AS), single stranded DNA, or a combination thereof.
7. The assay mixture of any one of statements 3-6, wherein the polymer comprises a linker that is covalently linked to the crRNA 5′-end and a segment that reduces the Cas nuclease activity.
8. The assay mixture of statement 7, wherein the linker comprises a 6-10 nucleotide single-stranded DNA.
9. The assay mixture of any of statements 2-8, wherein the Cas nuclease is a Cas13 nuclease, a Cas12 nuclease, or a combination of Cas13 nucleases and Cas12 nucleases.
10. The assay mixture of any of statements 2-9, wherein the Cas nuclease is a Cas13a nuclease, Cas13b nuclease, Cas13c nuclease, Cas13d nuclease, or a combination thereof.
11. The assay mixture of any one of statements 2-10, wherein the at least one CRISPR guide RNA (crRNA) binds to at least one of the target RNA(s).
12. The assay mixture of any one of statements 1-11, wherein the at least one target RNA comprises a viral RNA, a prokaryotic RNA, or a eukaryotic RNA.
13. The assay mixture of any one of statements 1-12, wherein the at least one target RNA comprises a wild type target RNA sequence.
14. The assay mixture of any one of statements 1-13, wherein the at least one target RNA comprises a variant or mutant target RNA sequence.
15. The assay mixture of any one of statements 1-14, wherein the at least one target RNA comprises one or more SARS coronaviruses (SARS-CoV-1 and/or SARS-CoV-2), Orthomyxoviruses (influenza viruses), Hepatitis C Viruses (HCVs), Ebola, influenza viruses, polio viruses, measles viruses, retroviruses, Human T-cell lymphotropic virus type 1 (HTLV-1), human immunodeficiency viruses (HIV), or a combination thereof.
16. The assay mixture of any one of statements 1-15, wherein the at least one target RNA comprises a coronavirus RNA.
17. The assay mixture of any one of statements 1-16, wherein the at least one target RNA comprises a mRNA for a disease marker.
18. The assay mixture of any one of statements 1-17, wherein the at least one target RNA comprises a microRNA.
19. The assay mixture of any one of statements 1-18, wherein the at least one reporter RNA comprises at least one fluorophore and at least one fluorescence quencher.
20 The assay mixture of statement 19, wherein the at least one fluorophore is Alexa 430, STAR 520, Brilliant Violet 510, Brilliant Violet 605, Brilliant Violet 610, or a combination thereof.
21. The assay mixture of any one of statements 1-20, wherein the population of droplets ranges in diameter from 20 to 60 μm.
22. The assay mixture of any one of statements 1-21, wherein different droplets comprise different CRISPR guide RNAs (crRNAs), each crRNA comprising RNA or an RNA-polymer hybrid.
23 The assay mixture of any one of statements 2-22, wherein the at least one CRISPR guide RNA (crRNA) in at least one droplet has a sequence comprising any one of SEQ ID NO: 1-69 or 70.
24. A method comprising measuring fluorescence of individual droplets in a population of droplets, the population comprising at least one droplet comprising a target RNA, a ribonucleoprotein complex, and at least one type of reporter RNA.
25 The method of statement 24, wherein the population comprises at least two, at least three, at least five, at least seven, at least ten, at least fifteen, at least twenty, at least thirty, at least fifty droplets, each comprising a target RNA, a ribonucleoprotein complex, and at least one type of reporter RNA.
26 The method of statement 24 or 25, wherein the target RNA is the same target RNA in the population of droplets.
27 The method of statement 24 or 25, wherein different droplets can contain or comprise a different target RNA.
28 The method of any one of statements 24-27, wherein each target RNA comprises a viral RNA, a prokaryotic RNA, or a eukaryotic RNA.
29 The method of any one of statements 24-28, wherein at least one target RNA comprises a wild type target RNA sequence.
30 The method of any one of statements 24-29, wherein the at least one target RNA comprises a variant or mutant target RNA sequence.
31. The method of any one of statements 24-30, wherein the at least one target RNA comprises one or more SARS coronaviruses (SARS-CoV-1 and/or SARS-CoV-2), Orthomyxoviruses (influenza viruses), Hepatitis C Viruses (HCVs), Ebola, influenza viruses, polio viruses, measles viruses, retroviruses, Human T-cell lymphotropic virus type 1 (HTLV-1), human immunodeficiency viruses (HIV), or a combination thereof.
32. The method of any one of statements 24-31, wherein the at least one target RNA comprises a coronavirus RNA.
33 The method of any one of statements 24-30, wherein the at least one target RNA comprises a mRNA for a disease marker.
34 The method of any one of statements 24-30, wherein the at least one target RNA comprises a microRNA.
35 The method of any one of statements 24-34, wherein the ribonucleoprotein complex comprises at least one Cas nuclease and at least one CRISPR guide RNA (crRNA).
36 The method of any one of statements 24-35, wherein one or more individual droplets comprise a CRISPR guide RNA (crRNA) comprising or consisting essentially of an crRNA-polymer hybrid, wherein a polymer is covalently linked to the 5′-end of at least one crRNA.
37. The method of statement 36, wherein the polymer inhibits cleavage of at least one of the reporter RNAs.
38 The method of statement 36 or 27, wherein the polymer reduces folding or activity by the Cas nuclease higher-eukaryotes-and-prokaryotes nucleotide-binding (HEPN) domain.
39 The method of any one of statements 36-38, wherein the polymer comprises polyethylene glycol, poly[oxy(11-(3-(9-adeninyl)propionato)-undecanyl-1-thiomethyl)ethylene] (PECH-AP), poly[oxy(11-(5-(9-adenylethyloxy)-4-oxopentanoato)undecanyl-1-thiomethyl)ethylene] (PECH-AS), single stranded DNA, or a combination thereof.
40. The method of any one of statements 36-39, wherein the polymer comprises a linker that is covalently linked to the crRNA 5′-end and a segment that reduces the Cas nuclease activity.
41. The method of statement 40, wherein the linker comprises a 6-10 nucleotide single-stranded DNA.
42. The method of any one of statements 24-41, wherein the at least one CRISPR guide RNA (crRNA) binds to at least one of the target RNA(s).
43. The method of any one of statements 24-42, wherein the Cas nuclease is a Cas13 nuclease, a Cas12 nuclease, or a combination of Cas13 nucleases and Cas12 nucleases.
44. The method of any one of statements 24-43, wherein the Cas nuclease is a Cas13a nuclease, Cas13b nuclease, Cas13c nuclease, Cas13d nuclease, or a combination thereof.
45. The method of any one of statements 24-44, wherein the at least one reporter RNA comprises at least one fluorophore and at least one fluorescence quencher.
46. The method of statement 45, wherein the at least one fluorophore is Alexa 430, STAR 520, Brilliant Violet 510, Brilliant Violet 605, Brilliant Violet 610, or a combination thereof.
47. The method of any one of statements 24-46, wherein the population of droplets ranges in diameter from 20 to 60 μm.
48. The method of any one of statements 24-47, wherein different droplets comprise different CRISPR guide RNAs (crRNAs), each crRNA comprising RNA or an RNA-polymer hybrid.
49 The method of any one of statements 24-48, wherein the at least one CRISPR guide RNA (crRNA) in at least one droplet has a sequence comprising any one of SEQ ID NO: 1-69 or 70.
50. The method of any one of statements 24-49, further comprising determining one or more of the following kinetic parameters: a slope of signal over time (slope), a time from target addition to the initiation of enzyme activity (Tmit), a root-mean-square-deviation (RMSD) of signal over time by linear regression for one or more of the individual droplets that emit signal.
51. The method of statement 50, further comprising determining a slopefast parameter for one or more of the individual droplets, where the slopefast
52. parameter comprises a percent of time where a signal slope is steep. The method of statement 50 or 51, further comprising determining a slopeslow parameter for one or more of the individual droplets, where the slopeslow parameter comprises a percent of time where a signal slope over time is shallow.
53. The method of any one of statements 24-52, further comprising before measuring fluorescence of individual droplets: (a) contacting a sample with at least one type of ribonucleoprotein complex and at least one type of reporter RNA to form a reaction mixture; (b) mixing the reaction mixture with oil and surfactant to form an emulsion comprising water-in-oil droplets, where at least some of the droplets encapsulate all components of the reaction mixture; and measuring fluorescence of individual droplets.
54 The method of statement 53, further comprising c) removing excess oil from the droplets before measuring fluorescence of individual droplets.
55. A method comprising (a) contacting a sample with at least one type of ribonucleoprotein complex and at least one type of reporter RNA to form a reaction mixture; (b) mixing the reaction mixture with oil and surfactant to form an emulsion comprising water-in-oil droplets, where at least some of the droplets encapsulate all components of the reaction mixture; (c) removing excess oil from the droplets; (d) selecting at least 1 droplet, or at least 3 droplets, or at least 10 droplets that emit fluorescence as positive droplets for monitoring; and (e) monitoring the fluorescence of the positive droplets over time.
56. The method of statement 55, further comprising determining one or more of the following kinetic parameters: a slope of signal over time (slope), a time from target addition to the initiation of enzyme activity (Tinit), a root-mean-square-deviation (RMSD) from signal time trajectories by linear regression for one or more of the positive droplets.
57. The method of statement 55 or 56, further comprising determining a slopefast parameter for one or more of the positive droplets, where the slopefast
58. parameter comprises a percent of time where a fluorescence slope is steep. The method of any one of statements 55-57, further comprising determining a slopeslow parameter for one or more of the positive droplets, where the slopeslow parameter comprises a percent of time where a fluorescence slope over time is shallow.
59. The method of any one of statements 55-58, wherein the sample comprises one or more target RNA(s).
60 The method of any one of statements 55-59, further comprising identifying what target RNA(s) are present the sample.
61. The method of any one of statements 55-60, wherein the at least one ribonucleoprotein complex comprises at least one Cas nuclease and at least one CRISPR guide RNA (crRNA).
62. The method of any one of statements 55-61, wherein the Cas nuclease is a Cas13 nuclease, a Cas12 nuclease, or a combination of Cas13 nucleases and Cas12 nucleases.
63. The method of any one of statements 55-62, wherein the at least one CRISPR guide RNA (crRNA) binds to at least one of the target RNA(s).
64 The method of any one of statements 55-63, wherein the at least one target RNA comprises a viral RNA, a prokaryotic RNA, or a eukaryotic RNA.
65 The method of any one of statements 55-64, wherein the at least one target RNA comprises sequence that hybridizes to a wild type target RNA sequence.
66 The method of any one of statements 55-65, wherein the at least one target 66. RNA comprises sequence that hybridizes to a variant or mutant target RNA sequence.
67 The method of any one of statements 55-66, wherein the at least one target RNA comprises a coronavirus RNA.
68. The method of any one of statements 55-67, wherein the at least one target RNA comprises a mRNA for a disease marker.
69 The method of any one of statements 55-68, wherein the at least one target RNA comprises a microRNA.
70 The method of any one of statements 55-69, wherein the at least one reporter RNA comprises at least one fluorophore and at least one fluorescence quencher.
71 The method of statement 70, wherein the at least one fluorophore is Alexa 430, STAR 520, Brilliant Violet 510, Brilliant Violet 605, Brilliant Violet 610, or a combination thereof.
72. The method of any one of statements 55-71, wherein the droplets ranges in diameter from 20 to 60 μm.
73. The method of any one of statements 55-72, wherein the reaction mixture comprises more than one ribonucleoprotein complex.
74. The method of any one of statements 55-73, wherein the reaction mixture comprises a mixture of different CRISPR guide RNAs (crRNAs).
75 The method of any one of statements 55-74, wherein at least one type of ribonucleoprotein complex comprises a CRISPR guide RNA (crRNA) comprising or consisting essentially of an RNA-polymer hybrid, wherein a polymer is covalently linked to the 5′-end of at least one crRNA.
76 The method of statement 75, wherein the polymer inhibits cleavage of at least one of the reporter RNAs.
77 The method of statement 75 or 76, wherein the polymer reduces folding and/or activity by the Cas nuclease higher-eukaryotes-and-prokaryotes nucleotide-binding (HEPN) domain.
78 The method of any one of statements 75-77, wherein the polymer comprises polyethylene glycol, poly[oxy(11-(3-(9-adeninyl)propionato)-undecanyl-1-thiomethyl)ethylene] (PECH-AP), poly[oxy(11-(5-(9-adenylethyloxy)-4-oxopentanoato)undecanyl-1-thiomethyl)ethylene] (PECH-AS), single stranded DNA, or a combination thereof.
79 The method of any one of statements 75-78, wherein the polymer comprises a linker that is covalently linked to the crRNA 5′-end and a segment that reduces the Cas nuclease activity.
80 The method of statement 79, wherein the linker comprises a 6-10 nucleotide single-stranded DNA.
81. The method of any one of statements 61-80, wherein the at least one CRISPR guide RNA (crRNA) has a SEQ ID NO:1-69 or 70 sequence.
82 The method of any one of statements 55-81, wherein the sample is an environmental sample (water, sewage, soil, waste, manure, liquids, or combinations thereof) or a sample from at least one animal.
83. The method of any one of statements 55-81, wherein the sample comprises bodily fluids, excretions, tissues, or combinations thereof from one or more animals.
84 The method of statement 82 or 83, wherein the one or more animals are one or more humans, birds, mammals, domesticated animals, zoo animals, wild animals, or combinations thereof.
85. An CRISPR guide RNA (crRNA)-polymer hybrid comprising a polymer covalently linked to the 5′-end of the crRNA.
86. The CRISPR guide RNA (crRNA)-polymer hybrid of statement 85, wherein the polymer inhibits cleavage by a ribonucleoprotein complex of a cas nuclease and the CRISPR guide RNA (crRNA)-polymer hybrid.
87. The CRISPR guide RNA (crRNA)-polymer hybrid of statement 85 or 86, wherein the polymer reduces folding, formation, or activity of a higher-eukaryotes-and-prokaryotes nucleotide-binding (HEPN) domain of a Cas nuclease in a ribonucleoprotein complex with the CRISPR guide RNA (crRNA)-polymer hybrid.
88. The CRISPR guide RNA (crRNA)-polymer hybrid of any one of statements 85-87, wherein the polymer comprises polyethylene glycol, poly[oxy(11-(3-(9-adeninyl)propionato)-undecanyl-1-thiomethyl)ethylene] (PECH-AP), poly[oxy(11-(5-(9-adenylethyloxy)-4-oxopentanoato)undecanyl-1-thiomethyl)ethylene] (PECH-AS), single stranded DNA, or a combination thereof.
89 The CRISPR guide RNA (crRNA)-polymer hybrid of any one of statements 85-88, wherein the polymer comprises a linker that is covalently linked to the crRNA 5′-end and a segment that reduces the Cas nuclease activity.
90 The CRISPR guide RNA (crRNA)-polymer hybrid of statement 89, wherein the linker comprises a 6-10 nucleotide single-stranded DNA.
91 A ribonucleoprotein complex of a cas nuclease and the CRISPR guide RNA (crRNA)-polymer hybrid of any one of statements 85-89.
92 A ribonucleoprotein complex of a cas nuclease and CRISPR guide RNA (crRNA)-polymer hybrid, wherein the CRISPR guide RNA (crRNA)-polymer comprises a polymer covalently linked to the 5′-end of the crRNA.

The specific methods and compositions described herein are representative of preferred embodiments and are exemplary and not intended as limitations on the scope of the invention. Other objects, aspects, and embodiments will occur to those skilled in the art upon consideration of this specification and are encompassed within the spirit of the invention as defined by the scope of the claims It will be readily apparent to one skilled in the art that varying substitutions and modifications may be made to the invention disclosed herein without departing from the scope and spirit of the invention.

The invention illustratively described herein suitably may be practiced in the absence of any element or elements, or limitation or limitations, which is not specifically disclosed herein as essential. The methods and processes illustratively described herein suitably may be practiced in differing orders of steps, and the methods and processes are not necessarily restricted to the orders of steps indicated herein or in the claims.

As used herein and in the appended claims, the singular forms “a,” “an,” and “the” include plural reference unless the context clearly dictates otherwise. Thus, for example, a reference to “a nucleic acid” or “a protein” or “a cell” includes a plurality of such nucleic acids, proteins, or cells (for example, a solution or dried preparation of nucleic acids or expression cassettes, a solution of proteins, or a population of cells), and so forth. In this document, the term “or” is used to refer to a nonexclusive or, such that “A or B” includes “A but not B,” “B but not A,” and “A and B,” unless otherwise indicated.

Under no circumstances may the patent be interpreted to be limited to the specific examples or embodiments or methods specifically disclosed herein. Under no circumstances may the patent be interpreted to be limited by any statement made by any Examiner or any other official or employee of the Patent and Trademark Office unless such statement is specifically and without qualification or reservation expressly adopted in a responsive writing by Applicants.

The terms and expressions that have been employed are used as terms of description and not of limitation, and there is no intent in the use of such terms and expressions to exclude any equivalent of the features shown and described or portions thereof, but it is recognized that various modifications are possible within the scope of the invention as claimed. Thus, it will be understood that although the present invention has been specifically disclosed by preferred embodiments and optional features, modification and variation of the concepts herein disclosed may be resorted to by those skilled in the art, and that such modifications and variations are considered to be within the scope of this invention as defined by the appended claims and statements of the invention.

The invention has been described broadly and generically herein. Each of the narrower species and subgeneric groupings falling within the generic disclosure also form part of the invention. This includes the generic description of the invention with a proviso or negative limitation removing any subject matter from the genus, regardless of whether or not the excised material is specifically recited herein. In addition, where features or aspects of the invention are described in terms of Markush groups, those skilled in the art will recognize that the invention is also thereby described in terms of any individual member or subgroup of members of the Markush group.

What is claimed:

1. A method comprising measuring or monitoring fluorescence of individual droplets in a population of droplets, the population comprising at least one droplet comprising a target RNA, a ribonucleoprotein complex, and at least one type of reporter RNA.

2. The method of claim 1, wherein the population comprises at least two, at least three, at least five, at least seven, at least ten, at least fifteen, at least twenty, at least thirty, at least fifty droplets, each comprising a target RNA, a ribonucleoprotein complex, and at least one type of reporter RNA.

3. The method of claim 1, wherein the target RNA is the same target RNA in the population of droplets.

4. The method of claim 1, wherein different droplets can contain or comprise different target RNAs.

5. The method of claim 1, wherein each target RNA comprises a viral RNA, a prokaryotic RNA, or a eukaryotic RNA.

6. The method of claim 1, wherein at least one target RNA comprises a wild type target RNA sequence.

7. The method of claim 1, wherein the at least one target RNA comprises a variant or mutant target RNA sequence.

8. The method of claim 1, wherein the at least one target RNA comprises one or more SARS coronaviruses (SARS-CoV-1 and/or SARS-CoV-2), Orthomyxoviruses (influenza viruses), Hepatitis C Viruses (HCVs), Ebola, influenza viruses, polio viruses, measles viruses, retroviruses, Human T-cell lymphotropic virus type 1 (HTLV-1), human immunodeficiency viruses (HIV), or a combination thereof.

9. The method of claim 1, wherein the at least one target RNA comprises a coronavirus RNA.

10. The method of claim 1, wherein the at least one target RNA comprises an RNA for a disease marker.

11. The method of claim 1, wherein the at least one target RNA comprises a microRNA.

12. The method of claim 1, wherein the ribonucleoprotein complex in one or more individual droplets comprises at least one Cas nuclease and at least one CRISPR guide RNA (crRNA).

13. The method of claim 1, wherein the ribonucleoprotein complex in one or more individual droplets comprises a CRISPR guide RNA (erRNA) comprising or consisting essentially of an RNA-polymer hybrid, wherein a polymer is covalently linked to the 5′-end of at least one crRNA.

14. The method of claim 13, wherein the polymer inhibits cleavage of at least one of the reporter RNAs.

15. The method of claim 13, wherein the polymer reduces the folding, formation or activity of a Cas nuclease higher-eukaryotes-and-prokaryotes nucleotide-binding (HEPN) domain.

16. The method of claim 13, wherein the polymer comprises polyethylene glycol, poly[oxy(11-(3-(9-adeninyl)propionato)-undecanyl-1-thiomethyl)ethylene] (PECH-AP), poly[oxy(11-(5-(9-adenylethyloxy)-4-oxopentanoato)undecanyl-1-thiomethyl)ethylene] (PECH-AS), single stranded DNA comprising natural or unnatural linkages and/or natural or unnatural nucleotides, or a combination thereof.

17. The method of claim 13, wherein the polymer comprises a linker that is covalently linked to the crRNA 5′-end and a segment that reduces the Cas nuclease activity.

18. The method of claim 17, wherein the linker comprises a 6-10 nucleotide single-stranded DNA.

19. The method of claim 1, wherein the ribonucleoprotein complex binds to at least one of the target RNA(s).

20. The method of claim 12, wherein the Cas nuclease is a Cas13 nuclease, a Cas12 nuclease, or a combination of Cas13 nucleases and Cas12 nucleases.

21. The method of claim 12, wherein the Cas nuclease is a Cas13a nuclease, Cas13b nuclease, Cas13c nuclease, Cas13d nuclease, or a combination thereof.

22. The method of claim 1, wherein the at least one reporter RNA comprises at least one fluorophore and at least one fluorescence quencher.

23. The method of claim 22, wherein the at least one fluorophore is Alexa 430, STAR 520, Brilliant Violet 510, Brilliant Violet 605, Brilliant Violet 610, or a combination thereof.

24. The method of claim 1, wherein the droplets range in diameter from 10 μm to 60 μm.

25. The method of claim 1, wherein different droplets comprise different CRISPR guide RNAs (crRNAs), each crRNA comprising RNA or an RNA-polymer hybrid.

26. The method of claim 1, wherein the ribonucleoprotein complex in at least one droplet comprises a CRISPR guide RNA (crRNA) with a sequence comprising any one of SEQ ID NO: 1-69 or 70.

27. The method of claim 1, comprising measuring or monitoring fluorescence over time.

28. The method of claim 1, further comprising determining one or more of the following kinetic parameters: a slope of signal over time (slope), a time from target RNA or sample addition to the initiation of enzyme activity (Tinit), a root-mean-square-deviation (RMSD) of signal over time by linear regression for one or more of the individual droplets that emit signal.

29. The method of claim 28, further comprising determining a slopefast parameter for one or more of the individual droplets, where the slopefast parameter comprises a percent of time where a signal slope is steep.

30. The method of claim 28, further comprising determining a slopeslow parameter for one or more of the individual droplets, where the slopeslow parameter comprises a percent of time where a signal slope over time is shallow.

31. The method of claim 1, further comprising before measuring fluorescence of individual droplets: (a) contacting a sample with at least one type of ribonucleoprotein complex and at least one type of reporter RNA to form a reaction mixture; (b) mixing the reaction mixture with oil and surfactant to form an emulsion comprising water-in-oil droplets, where at least some of the droplets encapsulate all components of the reaction mixture; and measuring fluorescence of individual droplets.

32. An assay mixture comprising a population of droplets ranging in diameter from at least 10 μm to 60 μm, the population of droplets comprising a test droplet subpopulation comprising at least one ribonucleoprotein complex, plus at least one reporter RNA, plus at least one target RNA.

33. The assay mixture of claim 32, wherein the at least one ribonucleoprotein complex comprises at least one Cas nuclease and at least one CRISPR guide RNA (crRNA).

34. The assay mixture of claim 32, wherein at least one of the CRISPR guide RNAs (crRNAs) comprises or consists essentially of an RNA-polymer hybrid, wherein a polymer is covalently linked to the 5′-end of at least one crRNA.

35. The assay mixture of claim 34, wherein the polymer inhibits cleavage of at least one of the reporter RNAs.

36. The assay mixture of claim 34, wherein the polymer folding, formation, or activity of the Cas nuclease higher-eukaryotes-and-prokaryotes nucleotide-binding (HEPN) domain.

37. The assay mixture of claim 34, wherein the polymer comprises polyethylene glycol, poly[oxy(11-(3-(9-adeninyl)propionato)-undecanyl-1-thiomethyl)ethylene] (PECH-AP), poly[oxy(11-(5-(9-adenylethyloxy)-4-oxopentanoato)undecanyl-1-thiomethyl)ethylene] (PECH-AS), single stranded DNA, or a combination thereof.

38. The assay mixture of claim 34, wherein the polymer comprises a linker that is covalently linked to the crRNA 5′-end and a segment that reduces the Cas nuclease activity.

39. The assay mixture of claim 38, wherein the linker comprises a 6-10 nucleotide single-stranded DNA.

40. The assay mixture of claim 33, wherein the Cas nuclease is a Cas13 nuclease, a Cas12 nuclease, or a combination of Cas13 nucleases and Cas12 nucleases.

41. The assay mixture of claim 33, wherein the Cas nuclease is a Cas13a nuclease, Cas13b nuclease, Cas13c nuclease, Cas13d nuclease, or a combination thereof.

42. A method comprising (a) contacting a sample with at least one type of ribonucleoprotein complex and at least one type of reporter RNA to form a reaction mixture; (b) mixing the reaction mixture with oil and surfactant to form an emulsion comprising water-in-oil droplets, where at least some of the droplets encapsulate all components of the reaction mixture; (c) removing excess oil from the droplets; (d) selecting at least 1 droplet, or at least 3 droplets, or at least 10 droplets that emit fluorescence as positive droplets for monitoring; and (e) monitoring the fluorescence of the positive droplets over time.

43. An assay mixture comprising a population of droplets ranging in diameter from at least 10 μm to 60 μm, the population comprising a test droplet subpopulation comprising at least one ribonucleoprotein complex, plus at least one reporter RNA, plus at least one target RNA.

44. An CRISPR guide RNA (crRNA)-polymer hybrid comprising a polymer covalently linked to the 5′-end of the crRNA.

45. The CRISPR guide RNA (crRNA)-polymer hybrid of claim 44, wherein the polymer inhibits cleavage by a ribonucleoprotein complex of a cas nuclease and the CRISPR guide RNA (crRNA)-polymer hybrid.

46. The CRISPR guide RNA (crRNA)-polymer hybrid of claim 44, wherein the polymer reduces folding, formation or activity of a higher-eukaryotes-and-prokaryotes nucleotide-binding (HEPN) domain of a Cas nuclease in a ribonucleoprotein complex with the CRISPR guide RNA (crRNA)-polymer hybrid.

47. The CRISPR guide RNA (crRNA)-polymer hybrid of claim 44, wherein the polymer comprises polyethylene glycol, poly[oxy(11-(3-(9-adeninyl)propionato)-undecanyl-1-thiomethyl)ethylene] (PECH-AP), poly[oxy(11-(5-(9-adenylethyloxy)-4-oxopentanoato)undecanyl-1-thiomethyl)ethylene] (PECH-AS), single stranded DNA, or a combination thereof.

48. The CRISPR guide RNA (crRNA)-polymer hybrid of claim 44, wherein the polymer comprises a linker that is covalently linked to the crRNA 5′-end and a segment that reduces the Cas nuclease activity.

49. The CRISPR guide RNA (crRNA)-polymer hybrid of claim 48, wherein the linker comprises a 6-10 nucleotide single-stranded DNA.

50. A ribonucleoprotein complex comprising a cas nuclease and CRISPR guide RNA (crRNA)-polymer hybrid, wherein the CRISPR guide RNA (crRNA)-polymer comprises a polymer covalently linked to the 5′-end of the crRNA.

51. An assay mixture comprising the ribonucleoprotein complex of claim 50 and a target RNA.

52. A method comprising measuring or monitoring cleavage of a reporter RNA by at least one ribonucleoprotein complex comprising a cas nuclease and CRISPR guide RNA (crRNA)-polymer hybrid in the presence of a target RNA, wherein the CRISPR guide RNA (crRNA)-polymer comprises a polymer covalently linked to the 5′-end of the crRNA.

53. The method of claim 52, comprising measuring or monitoring cleavage of a reporter RNA by at least two, or at least three, or at least ten ribonucleoprotein complexes, each ribonucleoprotein complex comprising a cas nuclease and CRISPR guide RNA (crRNA)-polymer hybrid.

54. The method of claim 53, wherein at least two of the ribonucleoprotein complexes comprise CRISPR guide RNA (crRNA)-polymer hybrids with different cRNA sequences, different polymers, or a combination thereof.
